(19) United States
(12) Patent Application Publication
(10) Pub. No.: US 2024/0374734 A1
(43) Pub. Date: Nov. 14, 2024

(54) Immunomodulatory Combinations of Antigen and Drug-Lipid Conjugate

(71) Applicant: Integrated Nanotherapeutics Inc., Burnaby (CA)

(72) Inventors: Sam Chen, Burnaby (CA); Anne Nguyen, Burnaby (CA); Joshua Zaifman, Burnaby (CA)

(21) Appl. No.: 18/690,040

(22) PCT Filed: Sep. 7, 2022
(86) PCT No.: PCT/CA2022/051340

§ 371 Date: Mar. 7, 2024

Related U.S. Application Data
(60) Provisional application No. 63/241,625, filed on Sep. 8, 2021.

(51) Int. Cl. A61K 47/54; A61K 9/127; A61K 9/51; A61K 31/573; A61K 39/00; A61K 47/69; A61P 37/06

(52) U.S. Cl. CPC A61K 47/542; A61K 9/1271; A61K 9/5123; A61K 31/573; A61K 47/6911; A61K 47/6929; A61P 37/06; A61K 2039/55555; A61K 2039/575; A61K 2039/577

(57) ABSTRACT

An immunomodulatory combination including: (i) a lipid conjugate comprising an immunomodulatory agent covalently linked to a lipophilic moiety by a cleavable linkage or through a cleavable linker, and (ii) an antigen and/or one or more nucleic acids that encode the antigen. The antigen is a protein, polypeptide, peptide, lipoprotein, glycolipid, polynucleotide, or polysaccharide. The lipid conjugate and the antigen and/or the one or more nucleic acids that encode the antigen are formulated in separate delivery vehicles or co-formulated in the same delivery vehicle.

### TECHNICAL FIELD

The disclosure relates to combinations of antigen and an immunomodulatory agent-lipid conjugate for immunomodulation, for example to treat, prevent and/or ameliorate antigen-induced disorders.

### BACKGROUND

Antigen presenting cells (APCs) instruct the adaptive immune system. APCs take up and process exogenous (and self) antigens and load them onto MHC (or HLA molecules) on the cell surface which subsequently can be recognized by various classes of T lymphocytes to elicit an appropriate immune response. While engaging antigen-specific T cells through their T cell receptor recognizing the antigen:MHC complex, APCs deliver additional signals to these T cells depending on whether the antigen has been taken up in the presence of pro-inflammatory or homeostatic/regulatory signals. In the event the antigen was taken up in the presence of pro-inflammatory signals (e.g. from an infectious pathogen), APCs become mature/activated, often characterized by high surface levels of co-stimulatory molecules. These mature APCs deliver costimulatory signals to antigen-specific T cells to elicit downstream humoral and/or cellular immune responses to the antigen. In contrast, if the antigen is taken up in the absence of inflammatory cues, APCs remain immature or express coinhibitory molecules that dampen the antigen-specific T cell response to induce regulatory T cell responses to antigen. Therefore, APCs possess a central role in instructing antigen-specific immune responses.

Many pathological conditions are associated with undesirable or inappropriate antigen-specific immune responses. These immune responses are diverse and range from allergic diseases to autoimmune diseases to transplant rejection. Existing treatments involve systemic, non-discriminatory global suppression of the immune system and are associated with increased risk of infection and/or cancer and additional adverse effects of the immune suppressive agents. Furthermore, because none of these treatments are curative, chronic use of the treatment is required which further exacerbates adverse reactions and importantly, fail to fully control the disease.

Tolerizing APCs to induce tolerance to a specific antigen is another approach to treat antigen-induced disorders. Such an approach may avoid the drawbacks of non-specific suppression of the immune system associated with immunosuppressants. However, one of the major limitations of tolerizing APCs for therapeutic treatment is the need to remove APCs from a subject's body and tolerize ex vivo, and the limited trafficking of these APCs to relevant disease tissues/sites once re-transplanted.

Thus, there is a need in the art for an immunomodulatory treatment that addresses the shortcomings of known approaches to modify a subject's immune response to a specific antigen, e.g. to treat, prevent and/or ameliorate antigen-induced disorders.

### SUMMARY

The disclosure seeks to address one or more limitations of known art or to provide useful alternatives thereof.

Various embodiments disclosed herein relate to an immunomodulatory combination comprising: a lipid conjugate comprising an immunomodulatory agent covalently linked to a lipophilic moiety by a cleavable linkage or through a cleavable linker; and an antigen and/or one or more nucleic acids that encode the antigen, wherein the antigen is a protein, polypeptide, peptide, lipoprotein, glycolipid, polynucleotide, or polysaccharide, wherein the lipid conjugate and the antigen and/or the one or more nucleic acids that encode the antigen are formulated in separate delivery vehicles or co-formulated in the same delivery vehicle. In some embodiments, the delivery vehicles are lipid nanoparticles and/or liposomes. In some of these embodiments, the delivery vehicles are lipid nanoparticles that deliver to antigen presenting cells (APCs). In some embodiments, the antigen or the one or more nucleic acids encoding the antigen is entrapped within a lipid nanoparticle or liposome and has a net charge that is opposite a net charge of a lipid in the lipid nanoparticle, or the antigen is lipophilic and incorporated into a lipid compartment of a lipid nanoparticle or liposome. In other embodiments, the antigen is hydrophilic and entrapped in a liposome containing an aqueous core.

In some embodiments, the lipid conjugate is co-formulated in the same delivery vehicle as the antigen or the one or more nucleic acids that encode the antigen. The immunomodulatory combination may comprise two or more (e.g. two, three, or more than three) lipid conjugates. In some embodiments, the lipid conjugate is a first lipid conjugate, and the immunomodulatory combination further comprises a second lipid conjugate, wherein the second lipid conjugate comprises an immunomodulatory agent covalently linked to a lipophilic moiety by a cleavable linkage or through a cleavable linker, wherein the immunomodulatory agent of the second lipid conjugate is different from the immunomodulatory agent of the first lipid conjugate, wherein the first lipid conjugate and the second lipid conjugate are formulated in separate delivery vehicles or are co-formulated in the same delivery vehicle. In some such embodiments, the immunomodulatory agent of the second lipid conjugate targets a different immune pathway than the immunomodulatory agent of the first lipid conjugate. In some embodiments, the first lipid conjugate and the second lipid conjugate are co-formulated in the same delivery vehicle.

In various embodiments, the immunomodulatory combinations disclosed herein may be used in treatment of a subject having an antigen-induced disorder or undesired antigen-driven immune response, or for use in manufacture of a medicament for treating the subject. This disclosure therefore provides a method for treating a subject having an antigen-induced disorder or undesired antigen-specific immune response comprising administering an immunomodulatory combination defined herein to the subject, wherein the antigen, or the one or more nucleic acids encoding the antigen, and the lipid conjugate are administered together or sequentially.

According to another aspect of the disclosure, there is provided a vaccine formulation comprising: at least one prodrug comprising an immunomodulatory agent that is conjugated to a lipid moiety; and at least one antigen and/or a nucleic acid that encodes the antigen, wherein the at least one antigen is a protein, polypeptide, peptide, lipoprotein, glycolipid, polynucleotide, or polysaccharide, wherein the at least one prodrug and the antigen and/or the nucleic acid that encodes the antigen are formulated in separate delivery vehicles or co-formulated in the same delivery vehicles in the formulation.

According to another aspect of the disclosure, there is provided a method for treating a subject having an antigen-induced disorder or undesired antigen-driven immune response comprising: administering at least one prodrug comprising an immunomodulatory agent that is conjugated to a lipophilic moiety; and administering at least one antigen and/or a nucleic acid that encodes the antigen, wherein the at least one antigen is a protein, polypeptide, peptide, lipoprotein, glycolipid, polynucleotide, or polysaccharide, wherein the at least one prodrug and the antigen and/or the nucleic acid that encodes the antigen are formulated separately in delivery vehicles or co-formulated in the same delivery vehicle, and wherein the at least one prodrug and the at least one antigen and/or nucleic acid that encodes the antigen are administered together or sequentially.

According to another aspect of the disclosure, there is provided use a prodrug comprising an immunomodulatory agent that is conjugated to a lipophilic moiety to treat a subject having an antigen-induced disorder or undesired antigen-driven immune response in combination with at least one antigen and/or a nucleic acid that encodes the at least one antigen, wherein the at least one antigen is a protein, polypeptide, peptide, lipoprotein, glycolipid, polynucleotide, or polysaccharide, and wherein the at least one prodrug and the antigen and/or the nucleic acid that encodes the antigen are (i) co-formulated in the same delivery vehicles in an at least one formulation, or (ii) formulated in separate delivery vehicles for sequential administration or co-administration to the subject.

According to another aspect of the disclosure, there is provided a combination of a prodrug comprising an immunomodulatory agent that is conjugated to a lipophilic moiety and at least one antigen and/or a nucleic acid that encodes the at least one antigen to treat a subject having an antigen-induced disorder or undesired antigen-driven immune response, wherein the at least one antigen is a protein, polypeptide, peptide, lipoprotein, glycolipid, polynucleotide, or polysaccharide, and wherein the at least one prodrug and the antigen and/or the nucleic acid that encodes the antigen are (i) co-formulated in a delivery vehicle, or (ii) formulated in separate delivery vehicles for sequential administration or co-administration to the subject.

According to a further aspect of the disclosure, there is provided a formulation comprising: at least two prodrugs comprising an immunomodulatory agent that is conjugated to a lipophilic moiety, wherein the at least two prodrugs are formulated in separate delivery vehicles or co-formulated in the same delivery vehicles in the formulation, and wherein the at least two prodrugs are targeting different immune pathways. Optionally the formulation comprises at least one antigen and/or a nucleic acid that encodes the antigen, wherein the at least one antigen is a protein, polypeptide, peptide, lipoprotein, glycolipid, polynucleotide, or polysaccharide. The antigen or nucleic acid that encodes the antigen may be formulated in a delivery vehicle, including co-formulated with the two prodrugs in one delivery vehicle or formulated in a separate delivery vehicles thereof.

According to any one of the foregoing aspects of the disclosure, the delivery vehicles may be lipid nanoparticles.

According to any one of the foregoing aspects or embodiments of the disclosure, the at least one prodrug may be co-formulated in the same delivery vehicles with the antigen or the nucleic acid that encodes the antigen.

According to any one of the foregoing aspects or embodiments of the disclosure, two prodrugs may be present in the formulation and formulated separately or co-formulated in the same delivery vehicles.

According to any one of the foregoing aspects or embodiments of the disclosure, the immunomodulatory agent may be a tolerogenic or anti-inflammatory agent.

According to any one of the foregoing aspects or embodiments of the disclosure, the immunomodulatory agent may be an immunostimulant or an immunosuppressant.

Furthermore, according to any one of the foregoing aspects or embodiments of the disclosure, the immunomodulatory agent of the at least one prodrug may be selected from prednisone, budesonide, prednisolone, methylprednisolone, hydrocortisone, cortisone, betamethasone, budesonide, triamcinolone, flunisolide, beclomethasone, fluticasone, mometasone, fludrocortisone, flumethasone, triamcinolone acetonide, isoflupredone, corticosterone, desoxycortone acetate, desoxycortone enanthate, 11-deoxycorticosterone, 11-deoxycortisol, aldosterone, dexamethasone, calcitriol, acetylsalicylic acid, salicylate, mycophenolic acid, sirolimus, tacrolimus, cholecalciferol, calcifediol, alfacalcidol, calcipotriol, falecalcitriol, maxacalcitol, paricalcitol, doxercalciferol, 22-oxacalcitriol, tacalcitol, eldecalcitol, elocalcitol, inecalcitol, becocalcidiol, seocalcital, ergocalciferol, lexacalcitol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, Ethyl 2-((2-chlorophenyl) (hydroxy)methyl) acrylate, or combinations thereof.

According to any one of the foregoing aspects or embodiments of the disclosure, two or more antigens and/or nucleic acid encoding the antigens may be present in the formulation and formulated separately or co-formulated in the same delivery vehicles.

According to any one of the foregoing aspects or embodiments of the disclosure, the antigen may be a peptide, polypeptide or protein.

Other objects, features, and advantages of the present disclosure will be apparent to those of skill in the art from the following detailed description and figures.

### BRIEF DESCRIPTION OF THE DRAWINGS

FIG. 1A shows gated plots (gated on live cells) from pancreatic lymph nodes of mice assessed for APC markers, CD11b and CD11c, by flow cytometry 48 hours post-administration of DiO (3,3′-Dioctadecyloxacarbocyanine Perchlorate) labelled lipid nanoparticles (LNPs, DSPC/Chol/DSPE-PEG at 54/45/1 mol:mol). Mice received 2 injections, 24 hours apart, of LNP injected at a dose of 600 mg/kg i.p.

FIG. 1B shows counts of the DiO positive APCs (gated on CD11b+CD11c+ cells) in the pancreatic lymph nodes of the mice injected with the DiO-labelled LNPs.

FIG. 1C shows gated plots (gated on live cells) from pancreatic islets of the mice assessed for APC markers, CD11b and CD11c, by flow cytometry 48 hours post-administration of DiO-labelled lipid nanoparticles (DSPC/Chol/DSPE-PEG at 54/45/1 mol:mol).

FIG. 1D shows counts of the DiO positive APCs (gated on CD11b+CD11c+ cells) in the pancreatic islets of the mice injected with the DiO-labelled LNPs.

FIG. 2A shows the DiO positive cell counts in islet macrophages (CD11b+CD11c+) from mice injected with PBS control, DSPC/Chol/DSPE-PEG LNP (DSPC/Chol/DSPE-PEG at 54/45/1 mol:mol) or ionizable LNP (A002/DSPC/Chol/PEG-DMG at 50/10/38.5/1.5 mol:mol). Mice were injected with 150 mg/kg of the LNP formulations loaded with DiO, and 24 hours later, islets, pancreatic lymph nodes and splenocytes were isolated.

FIG. 2B is a bar graph that depicts the percentage of DiO positive APCs (CD11b+CD11c+ cells) in islets, pancreatic lymph nodes and spleen, from the DSPC/Chol LNP (horizonal hatches) and the ionizable LNP-(diagonal hatches) injected mice.

FIG. 3 is a bar graph that shows the percentage of lipid conjugate (prodrug) remaining in LNPs (DSPC/Chol/Prodrug/DSPE-PEG at 49/40/10/1 mol:mol) co-formulated with dexamethasone (D045) and calcitriol (D053, D068, D083) lipid conjugates (prodrugs) after 2 hours incubation in human plasma (hPlasma).

FIG. 4 is a bar graph showing percentage of bone marrow dendritic cells (BMDCs) with co-stimulatory molecules on the surface of bone marrow dendritic cells (BMDCs) after aLNP lipid conjugate (prodrug) treatment. The LNPs were formulated with D034 or D045 dexamethasone lipid conjugates (prodrugs) and/or D053 and D083 calcitriol lipid conjugates (prodrugs) as indicated. The BMDCs were treated for 48 hours and the LNPs contained various calcitriol and dexamethasone lipid conjugates (alone or in combination). Subsequently, BMDCs were challenged with lipopolysaccharide (LPS) stimulation for 24 hours to determine whether lipid conjugate (prodrug) formulations could prevent LPS-mediated activation (i.e., tolerize BMDCs). The co-stimulatory markers of BMDCs were characterized as CD80-CD86-(vertical lines), CD80+CD86+dim (horizontal lines) and CD80+CD86+ (angled lines). Treatments are shown compared to BMDCs treated with LPS only (Control) and no LPS (Untreated).

FIG. 5 is a bar graph showing the percentage proliferation of CD4+ T cells for various LNP formulations of the pro-drugs of dexamethasone (INT-D034 and INT-D045) and calcitriol (INT-D053 and INT-D083) at mol % from 10 to 99% as indicated in a mixed leukocyte (MLR) reaction assay. Bone marrow derived dendritic cells (BMDCs) from C57Bl/6 mice were first treated with LNP containing various mol % of the dexamethasone or calcitriol conjugates for 48 hours and then activated by incubation with LPS for 24 hours. They were then harvested and mixed with CD4+ T cells isolated from Balb/cJ mice (Jackson Laboratories) at 5:1 or 10:1 T-to-BMDC ratio. Treatments are shown compared to BMDCs treated with LPS and empty LNP (Control), with LPS and no LNP (+LPS−LNP) and no LPS no LNP (Untreated).

FIG. 6 is a bar graph showing percentage proliferation of T cells for various lipid conjugate (prodrug) LNPs formulated with dexamethasone (D034, D045) and calcitriol (D083) lipid conjugates (prodrugs) and combinations of these prodrugs. C57Bl/6 BMDCs were treated for 48 hours with LNP (DSPC or DMPC, cholesterol, prodrugs, and PEG-DSPE (in molar ratio of 49/40/10/1) that contained a single lipid conjugate (prodrug) or a combination of lipid conjugates (prodrugs) and subsequently washed and co-cultured with Balb/c CD4+ T cells. The lipid concentration was 30 μM for all treatments. Data are expressed as mean+SD of % proliferation (via CFSE dilution) amongst CD4+ cells from triplicates.

FIG. 7 is a bar graph showing T cell proliferation as a function of different ratios of dexamethasone (D045) and calcitriol (D083) lipid conjugates (prodrugs) co-formulated in LNPs. Different ratios of dexamethasone (D045) and calcitriol (D083) lipid conjugates (prodrugs) in LNP impact T cell proliferation. C57BL/6 BMDCs were treated for 48 hours with LNP that contained various molar ratios of D045 and D083 and subsequently washed and co-cultured with Balb/c CD4+ T cells. The lipid concentration was 30 uM for all treatments. Data are expressed as mean+SD of % proliferation (via CFSE dilution) amongst CD4+ cells from triplicates.

FIG. 8 is a bar graph showing T cell proliferation of CD4+ T cells from OT-II mice after mixing with BMDCs pre-treated with LNPs formulated with the lipid conjugate containing calcitriol (D053), untreated or control LNP (Ctr LNP) at the concentrations indicated in the legend. Following LNP pre-treatment, BMDCs were pulsed with varying concentration of free Ovalbumin 323-339 peptide (OVA) and co-cultured with CD4+ T cells. Data are expressed as mean+SD of % proliferation (via CFSE dilution) amongst CD4+ cells from triplicates.

FIG. 9 is a bar graph showing T cell proliferation of CD4+ T cells collected from OT-II mice after mixing with C57BL/6 BMDCs treated with/without OVA, with control LNP, LNP encapsulating OVA or LNP coformulated with calcitrol and OVA (D053-LNP-OVA). Data are expressed as mean+SD of % proliferation (via CFSE dilution) amongst CD4+ cells from triplicates.

FIGS. 10A-M show structures of the exemplary immunomodulatory agent-lipid conjugates.

FIG. 11 is a bar graph of antigen specific T cell proliferation in a co-culture of Ova specific CD4+ T cells (from OTII mice) and C57BL/6 BMDCs pretreated with LNPs separately loaded with Ova mRNA and lipid conjugates. Error bars represent mean+SD.

FIG. 12 is a series of bar graphs showing proliferation of CFSE-labelled Ova specific CD4+ T cells (from OTII mice) after co-culture with C57BL/6 BMDCs pre-treated with various LNPs coloaded with Ova antigen-encoding mRNA and lipid conjugates of the invention (prodrugs). Data are shown as mean+SD of marker positive cells as a percent of CD4+ cells (Y axis).

FIGS. 13A and 13B are a set of bar graphs showing cytokine measured in supernatant of CFSE-labelled OTII CD4+ T cells co-cultured with C57BL/6 BMDCs pre-treated with LNPs coloaded with Ova antigen-encoding mRNA and lipid conjugates of the invention (prodrugs). Data are shown as mean+SD of marker positive cells as a percent of CD4+ cells (Y axis).

FIG. 14 is a bar graph showing antibody production in mice in response to injection of LNPs carrying Ova mRNA versus LNPs further loaded with lipid conjugate D034. Error bars represent ±SD, statistical analyses performed by one way ANOVA with Tukey's multiple comparison test, *P<0.5, ****P<0.0001, n≥7 mice per group from two independent experiments.

### DETAILED DESCRIPTION

Provided herein are immunomodulatory combinations (alternatively referred to as vaccine formulations), uses thereof and methods thereof. The immunomodulatory combination comprises (i) a lipid conjugate comprising an immunomodulatory agent linked to a lipophilic moiety, and formulated in a delivery vehicle (e.g. a lipid nanoparticle (LNP), liposome, or the like), and (ii) an antigen, or one or more nucleic acids that encode the antigen, formulated in a delivery vehicle. The lipid conjugate and the antigen or nucleic acid encoding same may be formulated in separate delivery vehicles or may be co-formulated in the same delivery vehicle.

In some embodiments, the immunomodulatory agents are taken up by APCs as demonstrated by either an in vitro assay or an in vivo assay. To illustrate, and without being limiting, this disclosure shows that immunomodulatory combinations disclosed herein can induce tolerizing phenotypes in myeloid cells (e.g., bone marrow dendritic cells (BMDC)). Furthermore, these tolerizing APCs can suppress antigen-specific T cell proliferation, induce antigen-specific T regulatory cells, reduce Th1 and Th2 cytokine secretion and reduce antigen-specific antibody production.

### Definitions

“Vaccine formulation” refers to any pharmaceutical formulation that comprises one or more of the same or different delivery vehicles as described herein to treat, prevent and/or ameliorate an antigen-induced disorder in a subject. The term includes formulations prepared in any suitable pharmaceutically acceptable salt and/or excipient.

“Immunomodulatory agent” refers to an agent that can alter an immune response in a subject. In one non-limiting embodiment, the immunomodulator is an immunostimulant that enhances an immune response in a subject. In another non-limiting embodiment, the immunomodulator is an immunosuppressant that prevents or reduces an immune response in a subject. Immunomodulators can regulate myeloid cells (monocytes, macrophages, dendritic cells, megakaryocytes and granulocytes) or lymphoid cells (T cells, B cells and Natural Killer (NK) cells) and any further differentiated cells thereof.

“Tolerogenic agent” refers to an agent that suppresses an immune response or induces tolerance to an antigen. In some embodiments, the tolerogenic agent improves suppression of an immune response to an antigen and/or improves induction of tolerance to an antigen. For example, the tolerogenic agent may promote tolerogenic presentation of the antigen by APCs.

“Antigen-induced disorder or undesired antigen-driven immune response” refers to a condition in a subject associated with antigen-specific immune stimulation, such as stimulation of the immune system against an antigen by antigen presenting cells. Such disorders include any unwanted stimulation of the immune system and include, without limitation, allergies, autoimmune diseases, transplant rejection, anti-drug antibody responses, and the like.

“Antigen” has the usual meaning in the art, and refers to a molecule or molecular complex that can bind to a specific antibody or T cell receptor to modify the immune system. In some embodiments, the antigen is a foreign or non-foreign protein, polypeptide, peptide, lipoprotein, glycolipid, polynucleotide, or polysaccharide. In some embodiments, the antigen induces the antigen-induced disorder.

“Delivery vehicle” refers to any suitable particle in which an immunomodulatory agent-lipid conjugate (e.g. a prodrug) can be formulated. Non-limiting examples include lipid nanoparticles, liposomes, and the like.

“Lipophilic moiety” with reference to a moiety linked to an immunomodulatory agent as part of lipid conjugate (e.g. a prodrug) or a lipophilic moiety of an ionizable or permanently charged lipid includes, without limitation, a lipid or other lipophilic group that imparts sufficient hydrophobicity to the immunomodulatory agent, prodrug or lipid to facilitate formulation thereof into a suitable delivery vehicle.

“Scaffold moiety” refers to a hydrocarbon chain of a lipophilic moiety of a lipid conjugate, prodrug or an ionizable or permanently charged lipid upon which one or more hydrocarbon chains are linked via one or more respective biodegradable groups.

“Prodrug”, “lipid prodrug”, “immunomodulatory agent-lipid conjugate”, “lipid conjugate”, “drug-lipid conjugate” or “prodrug conjugate” as used herein refers to the immunomodulatory agent linked to the lipophilic moiety via any suitable linkage or linker, including covalent and non-covalent bonds. In some embodiments, the linkage or linker is covalently attached. The immunomodulatory agent may be activated upon release from the lipophilic moiety.

### Lipid Conjugates

The lipid conjugate comprises an immunomodulatory agent that is linked to a lipophilic moiety.

In some embodiments, the immunomodulatory agent is a tolerogenic agent that supresses an immune response or induces tolerance to an antigen. In some embodiments, the immunomodulatory agent is an immunosuppressant. In some embodiments, the immunomodulatory agent is an immunostimulant.

Immunomodulatory agents exert their immunomodulatory effects in a subject by targeting various molecules of upstream and downstream immune pathways. Upstream are targets directly modified by the agents, and downstream are the key pathways through which immunomodulatory agents inhibit inflammation. Many of these agents converge/overlap on the same downstream pathways. Upstream targets include, without limitation: Glucocorticoid receptor, mammalian target of rapamycin (mTOR), COX1/COX2 (by direct acetylation as by ASA or inhibition of expression as by salicylate), Vitamin D receptor, JAK1, JAK2, JAK3, TYK2, and Calcineurin. Downstream targets include, without limitation: inhibition of NF-kappaB complex expression/activity, inhibition of AP-1 expression/activity, inhibition of p38 MAP Kinase pathway, and inhibition of NFAT family.

Examples of immunomodulatory agents for inclusion in the lipid conjugate include a non-steroidal anti-inflammatory drug (NSAID), an inflammasome inhibitor, a Janus kinase (JAK) inhibitor, a corticosteroid, an mTOR inhibitor, a DMARD (disease-modifying antirheumatic drug), a calcineurin inhibitor, and/or a vitamin D receptor agonist. In some embodiments, each immunomodulatory agent is independently selected from an NSAID, an inflammasome inhibitor, a JAK inhibitor, a corticosteroid, an mTOR inhibitor, a DMARD, a calcineurin inhibitor, or a vitamin D receptor agonist. In some embodiments, each immunomodulatory agent is independently selected from dexamethasone, calcitriol, acetylsalicylic acid, salicylate, mycophenolic acid, sirolimus, tacrolimus, cholecalciferol, calcifediol, alfacalcidol, calcipotriol, falecalcitriol, maxacalcitol, paricalcitol, doxercalciferol, 22-oxacalcitriol, tacalcitol, eldecalcitol, elocalcitol, inecalcitol, becocalcidiol, seocalcital, ergocalciferol, lexacalcitol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, or Ethyl 2-((2-chlorophenyl) (hydroxy)methyl) acrylate.

In one embodiment, the immunomodulatory agent is selected from dexamethasone, calcitriol, acetylsalicylic acid, mycophenolic acid, sirolimus and/or tacrolimus.

In another embodiment, the immunomodulatory agent is a JAK Inhibitor, a DMARD (disease-modifying antirheumatic drug), an NSAID, an Inflammasome inhibitor and/or a vitamin D receptor agonist.

For example, a JAK Inhibitor may be selected from filgotinib, baricitinib, tofacitinib, ruxolitinib, upadacitinib, oclacitinib, peficitinib, fedratinib, delgocitinib, deucravacitinib and/or abrocitinib.

The DMARD may be selected from auranofin, apremilast, azathioprine, chloroquine, hydroxychloroquine, ciclosporin, leflunomide, methotrexate, minocycline and/or sulfasalazine.

Examples of NSAIDs that may be incorporated into a lipid conjugate include salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin and/or licofelone.

An inflammasome inhibitor for incorporation in a lipid conjugate includes MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124 and/or Ethyl 2-((2-chlorophenyl) (hydroxy)methyl) acrylate.

Examples of vitamins D receptor agonists include calcitriol, cholecalciferol, calcifediol, alfacalcidol, calcipotriol, falecalcitriol, maxacalcitol, paricalcitol, doxercalciferol, 22-oxacalcitriol, tacalcitol, eldecalcitol, elocalcitol, inecalcitol, becocalcidiol, seocalcital, ergocalciferol and/or lexacalcitol.

The lipophilic moiety of the lipid conjugate imparts sufficient hydrophobicity to the immunomodulatory agent to facilitate formulation thereof in a suitable delivery vehicle. Examples of suitable lipid moieties include those described in co-owned and co-pending WO 2020/191477 (PCT/CA2020/000039; incorporated herein by reference).

Two or more lipid conjugates comprising immunomodulatory agents may be formulated in the same or separate delivery vehicles. In such embodiment, the two or more immunomodulatory agents may be formulated so that the two (or more) agents are stably retained within the same delivery vehicle at molar ratios that are additive or synergistic. For example, 2, 3, 4, or more than 4 lipid conjugates (e.g. 2, 3, 4, or more than 4 prodrugs) may be formulated together or in separate delivery vehicles. In some embodiments, the immunomodulatory combination comprises a plurality of lipid conjugates, wherein the plurality of lipid conjugates comprise 3, 4, 5, 6, 7, 8, 9, 10, or more than 10 immunomodulatory agents, wherein each immunomodulatory agent of the plurality of lipid conjugates is different, and wherein each lipid conjugate of the plurality of lipid conjugates is independently formulated in a separate delivery vehicle from the other components of the immunomodulatory combination or is co-formulated with one or more of the other components of the immunomodulatory combination. Additive or synergistic effects between the two or more immunomodulatory agents may be determined by any suitable technique, including the Chou Talalay method known to those of skill in the art. In some embodiments, the two lipid conjugates (e.g. the two prodrugs) are co-formulated in the same delivery vehicle. The lipid conjugates herein are particularly amenable to formulation in delivery vehicles at high encapsulation efficiencies, such as up to 90% encapsulation efficiency or more.

In some embodiments, the lipid conjugate is a first lipid conjugate, and the immunomodulatory combination further comprises a second lipid conjugate, wherein the second lipid conjugate comprises an immunomodulatory agent covalently linked to a lipophilic moiety by a cleavable linkage or through a cleavable linker, wherein the immunomodulatory agent of the second lipid conjugate is different from the immunomodulatory agent of the first lipid conjugate, wherein the first lipid conjugate and the second lipid conjugate are formulated in separate delivery vehicles or are co-formulated in the same delivery vehicle. In some embodiments, the immunomodulatory agent of the second lipid conjugate targets a different immune pathway than the immunomodulatory agent of the first lipid conjugate. In some embodiments, the first lipid conjugate and the second lipid conjugate are co-formulated in the same delivery vehicle.

In yet further embodiments, the immunomodulatory combination further comprises a third lipid conjugate. In some embodiments, the immunomodulatory agent of the third lipid conjugate targets a different immune pathway than the immunomodulatory agent of the first and/or second lipid conjugate. In yet further embodiments, the immunomodulatory combination further comprises a more than four lipid conjugates.

The immunomodulatory agent may be covalently or non-covalently linked with lipid moieties such as fatty acids, glycerides, phospholipids or other hydrophobic moieties, including those produced by organic synthesis. Linkage of the lipophilic moiety to the immunomodulatory agent typically increases the hydrophobicity of the immunomodulatory agent.

The Log P of the lipid conjugate may be sufficient to impart a desired degree of hydrophobicity to the immunomodulatory agent. In one embodiment, the predicted Log P of the lipid conjugate is between 5 and 30, between 6 and 28 or between 7 and 25.

In some embodiments, the immunomodulatory agent is covalently linked to the lipophilic moiety. In some embodiments, the immunomodulatory agent is covalently linked to the lipophilic moiety by a cleavable linkage or through a cleavable linker.

The immunomodulatory agent may be bioactive when linked to the lipophilic moiety or bioactive upon cleavage therefrom after administration to a subject. In this regard, the lipid conjugate may comprise one or more biodegradable groups that are cleavable upon administration of the lipid conjugate to a subject.

The biodegradable groups may be independently selected from linkages comprising one or more functional groups selected from an ester, amide, amidine, hydrazone, disulfide, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea.

In one embodiment, the lipophilic moiety may be derived from a precursor fatty acid or other lipophilic molecule having, for example, 5 to 30 carbon atoms, 14 to 20 carbon atoms or 16 to 18 carbon atoms.

In another embodiment, the lipophilic moiety is a linear or branched lipophilic chain with up to 3, 4, 5 or 6 biodegradable groups. In one embodiment, at least one of the biodegradable groups is selected from at least one of an ester, amide, amidine, hydrazone, disulfide, ether, carbonate, carbamate, thionocarbamate and combinations thereof. In one embodiment, the biodegradable group is an ester that is cleavable by an esterase in vivo.

In certain embodiments, a lipid conjugate is formulated in a delivery vehicle comprises a linear or branched lipophilic moiety conjugated to the immunomodulatory agent, the lipophilic moiety having the structure of Formula I:

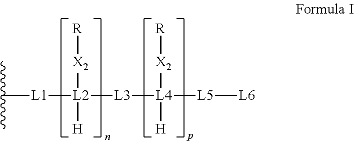

wherein the L is represented by L1+L2+L3+L4+L5+L6 and wherein L comprises 2 to 100, 2 to 75, 2 to 80, 3 to 60, 4 to 50, 5 to 45 or 5 to 40 carbon atoms and 0 to 6 cis or trans C═C double bonds;
wherein L1 is a carbon chain having 0 to 40, 1 to 40, 1 to 35, or 3 to 30 carbon atoms and optionally L1 has one or more cis or trans C═C double bonds or 0 to 2 cis or trans C═C double bonds;
wherein L2 and L4 are carbon atoms;
L3 is 0 to 20 carbon atoms and comprises 0 to 2 cis or trans C═C double bonds;
L5 is 0 to 20 carbon atoms and comprises 0 to 2 cis or trans C═C double bonds;
L6 is —CH₃, ═CH₂ or H;
each R is independently a linear or branched hydrocarbon chain having 0 to 30 carbon atoms and 0 to 3 cis or trans C═C double bonds, optionally 0 to 2 cis or trans C═C double bonds, wherein if one or more of R is branched, each branch point optionally includes an X2 functional group or is a carbon atom;
wherein n is 0 to 8 and p is 0 to 8, and wherein n+p is 0 or is ≥1 or 1 to 8, 2 to 6 or 2 to 4;
wherein each X2 if present is independently an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea;
or wherein X2 is a linkage that comprises at least one hydrogen bond.

In one embodiment, the lipid conjugate comprises a scaffold moiety. The scaffold moiety in one embodiment is represented by L of Formula I above and at least one R is present as a hydrocarbon side chain, wherein n+p is 1 or 1 to 8 or 1 to 7, or 1 to 6 or 1 to 5 or 1 to 4 or 1 to 3.

In some embodiments, the lipid conjugate comprises one lipophilic moiety, optionally having the structure of Formula I. In some embodiments, the lipid conjugate comprises two lipophilic moieties, each lipophilic moiety independently having the structure of Formula I, which may be the same or different, and each lipophilic moiety linked to the immunomodulatory agent through a separate linkage or linker, or linked to the same linkage or linker. In certain embodiments, each linkage is an ester. In some embodiments, the lipid conjugate comprises more than two lipophilic moieties.

In some embodiments, each lipophilic moiety of Formula I is independently defined by: L1 is a carbon chain having 3 to 30 carbon atoms, and 0 to 3 cis or trans C═C double bonds,
n is 0,
L3 is absent,
p is 1,
X2 is carbonate or ester

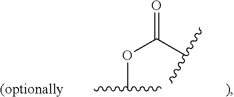

R is a linear or branched carbon chain having 1 to 20 carbon atoms and 0 to 3 cis or trans C═C double bonds,
L5 is a carbon chain having 1 to 10 carbon atoms and comprises 0 to 1 cis or trans C═C double bond, and
L6 is-CH₃;
or L is a carbon chain having 5 to 20 carbon atoms and zero C═C double bonds.

In some embodiments, each lipophilic moiety of Formula I is independently defined by: L1 is a carbon chain having 5 to 20 carbon atoms (optionally 8 to 15 carbon atoms), and 1 or 2 cis or trans C═C double bonds (optionally 1 cis or trans C═C double bond), optionally wherein L1 is —C₆₋₉—C═C—C—,
n is 0,
L3 is absent,
p is 1,
X2 is carbonate or ester

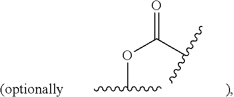

R is a linear or branched carbon chain having 1 to 20 carbon atoms and 0 to 2 cis or trans C═C double bonds (optionally wherein R is —C₁₋₆ or —C₅₋₈—C═C—C—C═C—C₄₋₆),
L5 is a carbon chain having 1 to 10 carbon atoms and comprises 0 to 1 cis or trans C═C double bond (optionally zero C═C double bonds), and
L6 is-CH₃;
or L is a carbon chain having 5 to 15 carbon atoms and zero C═C double bonds.

L1 may be linked to the immunomodulatory agent by a covalent linkage, or via hydrogen bonds, via an X1 linkage.

In some embodiments, the X1 linkage is biodegradable, meaning that it can be cleaved after administration to a subject. Without being limiting, an ester bond is capable of being hydrolyzed by an esterase after administration to a patient, thereby releasing the immunomodulatory agent from the lipophilic moiety. However, other X1 linkages can be utilized for tailored drug release based on their release characteristics when exposed to the environment at a disease site.

In some embodiments, X1 is cleavable by an esterase, alkaline phosphatase, amidase, peptidase or may be cleavable upon exposure to a reducing environment, and/or a high or low pH.

The X1 chemical linkage in certain embodiments is most advantageously a linker. A wide variety of chemical linkers is known to those of skill in the art and may be employed in certain embodiments described herein. A linker may have 0 to 12 carbon atoms and at least one cleavable functional group. In one embodiment, the linker has at least two functional groups, a first functional group for conjugating one end of the linker to the immunomodulatory agent and a second functional group for conjugating another end of the linker to a carbon atom on L of Formula I. The two functional groups may each be independently selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups such as an alkane, alkene or alkyne, methylene (CH₂) or urea.

The linker may provide enhanced release of the immunomodulatory agent through the introduction of a biodegradable group. A linker having one or more ester bonds may be capable of being hydrolyzed by an esterase after administration to a patient, thereby releasing the immunomodulatory agent from the lipid conjugate. Similar to a linkage resulting from direct reaction between the immunomodulatory agent and L, a linker introducing a hydrazone bond between the immunomodulatory agent and the lipophilic moiety can impart pH sensitive release the immunomodulatory agent from the lipid conjugate.

However, it will be understood that the foregoing is merely exemplary. Additional examples of linkers are provided in U.S. Pat. No. 5,149,794, which is incorporated herein by reference. Non-limiting examples of linkers described in U.S. Pat. No. 5,149,794 include aminohexanoic acid, polyglycine, polyamides, polyethylenes, and short functionalized polymers having a carbon backbone that is one to twelve carbon atoms in length.

Yet further examples of linkers suitable for use in the lipid conjugates described herein are provided in the following references:
1. Rautio et al., “The expanding role of prodrugs in contemporary drug design and development” Nature Reviews Drug Discovery 2018, 17, 559.
2. Irby et al., “Lipid-drug conjugate for enhancing drug delivery” Molecular Pharmaceutics 2017, 14, 1325.
3. Sun et al., “Chemotherapy agent-unsaturated fatty acid prodrugs and prodrug-nanoplatforms for cancer chemotherapy” Journal of Controlled Release 2017, 264, 145.
4. Walther et al., “Prodrugs in medicinal chemistry and enzyme prodrug therapies” Advanced Drug Delivery Reviews 2017, 118, 65.
5. Hu et al., “Glyceride-mimetic prodrugs incorporating self-immolative spacers promote lymphatic transport, avoid first-pass metabolism and enhance oral bioavailability” Angewandte Chemie International Edition 2016, 55, 13700.
6. Blencowe et al., “Self-immolative linkers in polymeric delivery systems” Polymer Chemistry 2011, 2, 773.

Each of the foregoing references is incorporated herein by reference in its entirety. In further embodiments, the X1 chemical linkage comprises both a functional group and a separate linker. Various combinations of linkers and functional groups can be incorporated into the lipid conjugate.

In one embodiment, at least the second functional group conjugating one end of the linker to L1 is an ester or an amide linkage. In another embodiment, a functional group on the linker can be hydrolyzed by an enzyme such as an esterase. In a further embodiment, both functional groups on the linker are ester linkages.

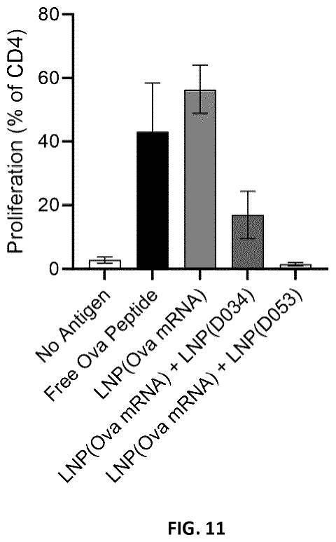

Several exemplary lipid conjugates are shown in FIGS. 10A-10M. Manufacture of these and other lipid conjugates for use in the immunomodulatory combinations disclosed herein is known in the art and has been previously described. For example, see WO/2020/191477, which is incorporated by reference herein in its entirety. Synthetic procedures for several lipid conjugates is described in the Examples.

Briefly, the immunomodulatory agent can be attached to the lipid moiety by conjugation to a reactive group on scaffold L or to a linker group to form chemical linkage X1. In one embodiment, the immunomodulatory agent loses a hydroxyl group or a hydrogen atom upon conjugation with the lipophilic moiety (e.g. Formula I) or a linker to form the lipid conjugate. The immunomodulatory agent may be derived from a chemical structure that contains one or more reactive functional groups such as —(C═O) O, OH, —NH₂, —NHR, —PO₃H₂, among others known to those of skill in the art, without limitation to the orientation of the atoms.

For example, the lipid conjugate may be formed (directly or via one or more intermediates) by a conjugation between a (C═O)OH group on the immunomodulatory agent and a hydroxyl group on precursor scaffold P. The general reaction is shown below for a molecule of interest (e.g. an immunomodulatory agent):

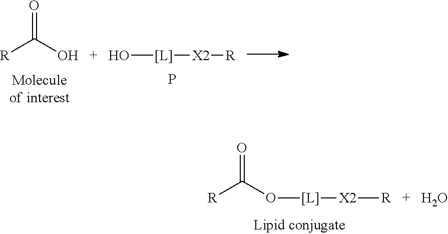

In the above exemplary embodiment, the X1 chemical linkage is an ester and has the following structure:

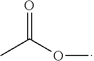

In another illustrative example, the immunomodulatory agent may have a hydroxyl group (—OH) that reacts with a carboxyl group ((C═O)OH) in a linker. A second carboxyl group ((C═O)OH) on the linker may react with a hydroxyl group on a carbon atom on a precursor scaffold P via a condensation reaction. The following reaction depicts the use of succinic acid as a linker. The use of such a linker results in a lipid conjugate that has two ester groups according to the following reaction:

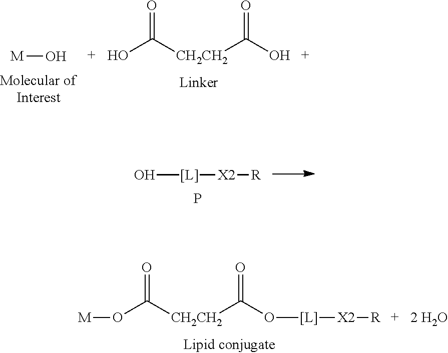

In the above non-limiting example, the X1 chemical linkage has the following structure:

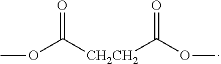

It should be appreciated that the above reaction may proceed in two steps. That is, the immunomodulatory agent may first be conjugated to the linker and the resultant drug-linker conjugate subsequently reacted with the precursor scaffold P to produce a lipid conjugate (prodrug) reaction product.

The foregoing is provided simply for illustrative purposes as a variety of different linkers besides succinic acid can be used to produce the lipid conjugates.

In another example, the immunomodulatory agent or a linker may have a carboxyl group ((C═O)O) for conjugation with an amine group of L to form an amide or amide-containing linkage X1 between the immunomodulatory agent and L. As discussed below, other reactions between functional groups on a drug or a linker with a scaffold L can be envisaged by those of skill in the art to produce an X1 chemical linkage.

Certain immunomodulatory agents may comprise more than one reactive functional group for linkage to precursor scaffold P. In such embodiments, a protecting group may be employed during the synthesis of the drug-lipid conjugate as would be appreciated by those of skill in the art to selectively conjugate a given group on the drug to the scaffold L and leave another group unconjugated.

### Antigen or Nucleic Acid(s) Encoding Antigen

In some embodiments, the antigen is a protein, polypeptide, peptide, lipoprotein, glycolipid, polynucleotide, or polysaccharide. In certain embodiments, the antigen is a protein, polypeptide or peptide. An antigen can be formulated in the delivery vehicles herein in the same form as that known to elicit an undesired immune response including, but not limited to, a fragment or derivative thereof. The antigen may originate from within the body of the subject, referred to as auto or “self,” or originate from the external environment, referred to as foreign or “non-self”.

The antigen includes, but is not limited to, an allergen, a superantigen, a tolerogen, a T-dependent antigen, a T-independent antigen or an immunodominant antigen. In another embodiment, the antigen is characterized by its source and includes an exogenous antigen, an endogenous antigen, an autoantigen or a neoantigen.

The antigen may have a single epitope, or may comprise more than one epitope (e.g. 2, 3, 4, 5, 6, 7, 8, 9, 10, 15, 20, 30, 40, 50, 60, 70, 80, 100, or more than 100 epitopes).

In some embodiments, the antigen is capable of being presented on an antigen presenting cell (APC) and activating T cells of the immune system using the delivery vehicles described herein.

In some embodiments, the antigen is an antigen associated with an allergic reaction, autoimmune disease, organ or tissue rejection, graft versus host disease, anti-drug antibody or gene/protein replacement therapy. One or more of these disorders may also be referred to as an inflammatory disorder. When the antigen is associated with an inflammatory reaction, the antigen may include but is not limited to a non-self antigen that is an allergen as described or a self or non-self antigen that elicits an unwanted immune response.

When the antigen is associated with an allergic reaction, the antigen may include, but is not limited to, a non-self antigen, also referred to as an allergen, originating from an animal source, including animal substances or foods from terrestrial or aqueous animals, plant sources, such as plant pollens or gluten, drugs, foods, insect stings, fungal sources, such as mold spores, metals, latex and the like.

Non-limiting examples of allergies include allergic asthma, hay fever, hives, eczema, plant allergies, insect sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. Non-limiting examples of food allergies include milk allergies, egg allergies, peanut/legume allergies, tree nut allergies, fish allergies, shellfish allergies, soy allergies and gluten allergies.

Examples of inflammatory diseases include Alzheimer's, arthritis, asthma, atherosclerosis, Crohn's disease, colitis, cystic fibrosis, dermatitis, diverticulitis, hepatitis, irritable bowel syndrome (IBS), lupus erythematous, muscular dystrophy, nephritis, for example glomerulonephritis Parkinson's, shingles and ulcerative colitis, cardiovascular disease, idiopathic pulmonary fibrosis, chronic obstructive pulmonary disease (COPD), bronchiectasis, chronic cholecystitis, tuberculosis, sepsis, sarcoidosis, silicosis and other pneumoconiosis, uveitis, orchitis, oophoritis, pancreatitis, gastritis, rheumatic fever.

Autoimmune diseases include, but are not limited to, rheumatoid arthritis, multiple sclerosis, myelin oligodendrocyte glycoprotein antibody disorder, primary biliary cholangitis, immune-mediated or Type I diabetes mellitus, systemic lupus erythematosus, psoriasis, scleroderma, autoimmune thyroid disease, such as Hashimoto's thyroiditis and primary myxoedema, alopecia areata, Grave's disease, Guillain-Barre syndrome, celiac disease, Sjogren's syndrome, autoimmune atrophic gastritis, autoimmune hepatitis, autoimmune pancreatitis, phacogenic uveitis, neuromyelitis optica myasthenia gravis, pernicious anemia, autoimmune haemolytic anemia, Addison's disease, scleroderma, Goodpasture's syndrome, anti-glomerular basement membrane disease, psoriasis, pemphigus vulgaris, pemphigoid, sympathetic opthalmia, thrombocytopenia purpura, autoimmune neutropenia, vitiligo, autoimmune vasculitis and dermatomyositis.

The antigen may also be used to treat, prevent and/or ameliorate a condition associated with an organ or tissue rejection. Such antigens include those derived from allogeneic cells, e.g., antigens from an allogeneic cell extract and antigens from other cells, such as endothelial cell antigens.

In further embodiments, the antigen may be used to treat, prevent and/or ameliorate an unwanted condition associated with a transplantable graft. Such antigens are associated with an undesired immune response in a recipient of a transplantable graft. In some embodiments, transplant antigens include those associated with organ or tissue rejection or graft versus host disease. Such antigens also may be obtained or derived from cells of a biological material or from information related to a transplantable graft. Transplant antigens generally include those contained or expressed in cells. Information related to a transplantable graft may include, but is not limited to, sequence information, types or classes of antigens and/or their MHC Class I, MHC Class II or B cell presentation restrictions. In other embodiments, the information used to produce an antigen includes the type of transplantable graft (e.g., autograft, allograft, xenograft), the molecular and cellular composition of the graft, the bodily location from which the graft is derived or to which the graft is to be transplanted (e.g., whole or partial organ, skin, bone, nerves, tendon, neurons, blood vessels, fat, cornea, etc.).

According to certain embodiments, the antigen is encoded by nucleic acid that is directly or indirectly capable of expression of an antigenic protein, peptide, polypeptide or fragments thereof. The antigen may be encoded by a nucleic acid sequence including, but not limited to DNA or RNA and hybrids thereof. The DNA includes any vector capable of expressing the antigen. The RNA may encode an mRNA or self-amplifying RNA that in turn encodes the antigen protein, peptide, polypeptide or fragments thereof.

The DNA encoding the antigen is typically circular (e.g., a plasmid, minicircle or nanoplasmid), although linearized DNA is also contemplated in certain embodiments herein. In some embodiments, the DNA encoding the antigen replicates autonomously. DNA that replicates autonomously will have an origin of replication or autonomous replicating sequence (ARS) that is functional in a host cell. In another embodiment, DNA encoding the antigen may replicate by being inserted into the genome of the host cell in a subject using known techniques.

The DNA encoding the antigen may encode regulatory regions such as promoter sequences, and termination regions. The DNA encoding the antigen can be cloned in an appropriate microorganism, (e.g., E. coli) and then formulated in the delivery vehicles disclosed herein for expression in vitro or in vivo. The DNA sequence may comprise a reporter gene sequence, although the inclusion of a reporter gene sequence in formulations for administration is optional. Such sequences may be incorporated into DNA for studies in animal models.

The DNA encoding the antigen may be single stranded, double-stranded or in some embodiments is a DNA-RNA hybrid. Single-stranded nucleic acids include antisense oligonucleotides (complementary to DNA and RNA), ribozymes and triplex-forming oligonucleotides. In order to have prolonged activity, the single-stranded nucleic acids in some embodiments may have some or all of the nucleotide linkages substituted with stable, non-phosphodiester linkages.

The DNA encoding the antigen may include nucleic acids in which modifications have been made in one or more sugar moieties and/or in one or more of the pyrimidine or purine bases. In further embodiments, the DNA vector may be modified with a peptide, protein, steroid or sugar moiety. Such modifications may facilitate delivery to a target site of interest.

The nucleic acids used in the immunomodulatory combination can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

As discussed previously, the nucleic acid may be RNA that encodes the antigen, such as mRNA or self-amplifying RNA. The RNA can be purified from natural sources, produced using recombinant expression systems and optionally purified, or may be chemically synthesized. In certain embodiments, the RNA encoding the antigen encompasses both modified and unmodified RNA.

In those embodiments in which an RNA is chemically synthesized, the RNA can comprise nucleoside analogs such as analogs having chemically modified bases or sugars, and/or backbone modifications. In some embodiments, an RNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, pseudouridine, 5-mythoxyuridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The RNA encoding the antigen may be synthesized according to any of a variety of known methods. For example, the RNA in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized RNA encoding the antigen may be purified before formulation and encapsulation to remove undesirable impurities including various enzymes and other reagents used during RNA synthesis.

In one embodiment, the RNA comprises one or more coding and non-coding regions.

RNA may be of a variety of lengths. In some embodiments, the present disclosure may be used to formulate in vitro synthesized RNA ranging from about 0.1-20 kb, 1-20 kb, about 1-15 kb, about 1-10 kb, about 5-20 kb, about 5-15 kb, about 5-12 kb, about 5-10 kb, about 8-20 kb, or about 8-15 kb in length. Some antigens may be as small as 8-12 amino acids long.

Typically, RNA synthesis includes the addition of a “cap” on the 5′ end, and a “tail” on the 3′ end. The presence of the cap is important in providing resistance to nucleases found in most eukaryotic cells. The presence of a “tail” serves to protect the RNA from exonuclease degradation.

In some embodiments, the RNA encoding the antigen includes a 5′ and/or 3′ untranslated region. In some embodiments, a 5′ untranslated region includes one or more elements that affect an RNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect the stability of an RNA in a location in a cell, or one or more binding sites for RNAs. In some embodiments, a 3′ untranslated region may be between 50 and 500 nucleotides in length or longer.

While RNA provided from in vitro transcription reactions may be desirable in certain embodiments, other sources of RNA are contemplated, such as RNA produced from bacteria, fungi, plants, and/or animals.

The RNA sequence may comprise a reporter gene sequence, although the inclusion of a reporter gene sequence in pharmaceutical formulations for administration is optional. Such sequences may be incorporated into RNA for in vivo studies in animal models to assess biodistribution.

In some embodiments, the immunomodulatory combination comprises a single antigen or one or more nucleic acids encoding the single antigen. In other embodiments, the immunomodulatory combination comprises more than one antigen or one or more nucleic acids encoding the more than one antigen. The immunomodulatory combination may comprise a combination of antigen(s) and nucleic acid(s) encoding antigen(s). In certain embodiments, the more than one antigen comprises two antigens. In certain embodiments, the more than one antigen comprises more than two antigens.

In certain embodiments, the immunomodulatory combination comprises an antigen or one or more nucleic acids that encode the antigen. In some of these embodiments, the antigen is a first antigen, and the immunomodulatory combination further comprises a second antigen or one or more nucleic acids encoding the second antigen, wherein the first antigen is different than the second antigen, wherein the second antigen or the one or more nucleic acids encoding the second antigen is formulated in a separate delivery vehicle from the other components of the immunomodulatory combination or is co-formulated with one or more of the other components of the immunomodulatory combination. In certain embodiments, the one or more nucleic acids encoding the first antigen and the one or more nucleic acids encoding the second antigen are comprised within a single nucleic acid and the first antigen and the second antigen are co-formulated in the same delivery vehicle.

In certain embodiments, the immunomodulatory combination comprises a plurality of antigens, or comprises one or more nucleic acids encoding the plurality of antigens, or comprises a combination of antigens and antigen-encoding nucleic acid(s) for providing the plurality of antigens. Each antigen or antigen-encoding nucleic acid is independently formulated in a separate delivery vehicle from the other components of the immunomodulatory combination or is co-formulated with one or more of the other components of the immunomodulatory combination. Each antigen may comprise one or more than one epitope. In alternative embodiments, the plurality of antigens comprises 3, 4, 5, 6, 7, 8, 9, 10, or more than 10 antigens.

### Delivery Vehicles

A variety of delivery vehicles can be used to prepare the immunomodulatory combinations (alternatively referred to as “vaccine formulations”). These include, but are not limited to, nanoparticles, including lipid nanoparticles (LNPs), liposomes, polymer nanoparticles comprising lipids, polymer-based nanoparticles, emulsions, and micelles.

The lipid conjugates of the present disclosure are particularly amenable to incorporation into nanoparticles (e.g. lipid nanoparticles, liposomes, or polymer-based systems) comprising lipids or other hydrophobic components. The lipid-like properties of the lipid conjugate in certain embodiments may facilitate its loading into these or other delivery vehicles. For example, in some embodiments, the loading efficiency into a given nanoparticle is 75% to 100%, 80% to 100% or most advantageously 90% to 100%. In some embodiments, the delivery vehicle(s) are liposomes and/or lipid nanoparticles.

In one embodiment, the lipid conjugates and antigen or nucleic acid encoding same are loaded into lipid nanoparticles or liposomes, by mixing them with lipid formulation components, including vesicle forming lipids and optionally a sterol. As a result, lipid nanoparticles and/or liposomes incorporating the cargo can be prepared using a wide variety of well described formulation methodologies known to those of skill in the art, including but not limited to extrusion, ethanol injection and in-line mixing. Such methods are described in Maclachlan, I. and P. Cullis, “Diffusible-PEG-lipid Stabilized Plasmid Lipid Particles”, Adv. Genet., 2005. 53PA: 157-188; Jeffs, L. B., et al., “A Scalable, Extrusion-free Method for Efficient Liposomal Encapsulation of Plasmid DNA”, Pharm Res, 2005. 22 (3): 362-72; and Leung, A. K., et al., “Lipid Nanoparticles Containing siRNA Synthesized by Microfluidic Mixing Exhibit an Electron-Dense Nanostructured Core”, The Journal of Physical Chemistry. C, Nanomaterials and Interfaces, 2012, 116 (34): 18440-18450, each of which is incorporated herein by reference in its entirety.

While liposomes comprise an aqueous internal solution surrounded by a phospholipid bilayer, a lipid nanoparticle may alternatively comprise a lipophilic core. Such lipophilic core can serve as a reservoir for the lipid conjugate (e.g. pro-drug) and/or the antigen or nucleic acid encoding same. Solid and liquid lipid nanoparticles can be used for the delivery of the lipid conjugate(s) and/or antigen(s) or nucleic acid(s) encoding same as described herein.

Provided in one embodiment is a liposome or lipid nanoparticle that comprises a phospholipid bilayer and wherein the lipid conjugate forms a hydrophobic oil phase within the bilayer. Such delivery vehicles are described in WO 2020/191477 (PCT/CA2020/000039), which is incorporated herein by reference. In another embodiment, the delivery vehicle is a liposome.

In some embodiments, at least one delivery vehicle is a lipid nanoparticle, optionally wherein at least one lipid conjugate is incorporated into a lipid compartment of the lipid nanoparticle. In some embodiments, at least one delivery vehicle is a liposome, optionally wherein at least one lipid conjugate is incorporated within the oily phase of the lipid bilayer of the liposome. In certain embodiments, at least one antigen or one or more nucleic acids encoding the at least antigen is entrapped within a lipid nanoparticle or liposome and has a net charge that is opposite a net charge of a lipid in the lipid nanoparticle. In certain embodiments, at least one antigen is lipophilic and incorporated into a lipid compartment of a lipid nanoparticle or liposome. In certain embodiments, at least one antigen is hydrophilic and entrapped in a liposome containing an aqueous core.

A delivery vehicle can also be a nanoparticle (e.g. liposome or LNP) that comprises a lipid core stabilized by a surfactant. Vesicle-forming lipids may be utilized as stabilizers. In another embodiment, a delivery vehicle is a polymer-lipid hybrid system that comprises a polymer nanoparticle core surrounded by stabilizing lipid.

Nanoparticles may alternatively be prepared from polymers without lipids. Such nanoparticles may comprise a concentrated core of drug that is surrounded by a polymeric shell or may have a solid or a liquid dispersed throughout a polymer matrix.

The lipid conjugates and/or antigen or nucleic acid encoding same described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

Micelles are self-assembling particles composed of amphipathic lipids or polymeric components that are utilized for the delivery of agents present in the hydrophobic core. Conjugating a drug to a scaffold molecule L and with a hydrophobic group R as described herein may improve drug loading into a micelle.

A further class of drug delivery vehicles known to those of skill in the art that can be used to encapsulate the lipid conjugate herein is carbon nanotubes.

Various methods for the preparation of the foregoing delivery vehicles and the incorporation of lipid-conjugated immunomodulatory agents therein are available and may be carried out with ease by those skilled in the art.

Certain lipid conjugates encompassed by the disclosure may form part of a carrier-free system. In such embodiments, the lipid conjugate can self-assemble into particles. Without being limiting, if the immunomodulatory agent is hydrophilic, then the amphiphilic pro-drug may assemble into nanoparticles with or without a stabilizer.

LNPs can be made using a wide variety of well described formulation methodologies including high pressure extrusion, ethanol injection, microfluidic mixing and in-line mixing.

In some embodiments, the polydispersity index (PdI) of the drug delivery vehicle comprising the lipid conjugate (prodrug) and/or the antigen or nucleic acid encoding same is less than 0.40, 0.35, 0.30, 0.25, 0.20 or 0.15.

The lipid conjugates described herein are particularly amenable to high encapsulation efficiency in drug delivery vehicles. In one embodiment, the encapsulation efficiency of the lipid conjugate (prodrug) is 10 to 99%, 15 to 99%, 20 to 99% or 25 to 99%.

The delivery vehicle comprising the lipid conjugate(s) may be co-formulated with the antigen(s) or nucleic acid(s) encoding same or the lipid conjugate(s) and antigen(s) or nucleic acid(s) may be formulated in separate delivery vehicles. In some embodiments, the lipid conjugate(s) is co-formulated in the same delivery vehicle as the antigen(s) or the one or more nucleic acids that encode the antigen(s).

The antigen or nucleic acid encoding same may be formulated in a delivery vehicle, such as a lipid nanoparticle, comprising an ionizable or permanently charged lipid. If an antigen that is a peptide, polypeptide or protein is encapsulated in a lipid nanoparticle, the ionizable or permanently charged lipid can be cationic or anionic depending on the charge of the antigen at physiological pH and temperature. The charged lipid may comprise a lipophilic moiety that comprises a scaffold and one or more hydrocarbon side chains linked thereto by biodegradable groups, for example as described in co-owned WO 2021/026647; Application No. PCT/CA2020/051098, which is incorporated herein by reference.

Charged antigens (e.g. proteins, peptides and/or nucleic acids) can be entrapped within liposomes or LNPs using ionic interactions. If the antigen is anionic, cationic lipids can be used and vice versa. If nucleic acid is formulated in the delivery vehicle, then the ionizable or permanently charged lipid is typically positively charged at physiological pH. Similarly, if the antigen is charged, the ionizable or permanently charged lipid will typically have a charge that is opposite to that of the antigen to facilitate its formulation in the lipid nanoparticle. For example, if the antigen is a peptide, polypeptide or protein that is charged, the ionizable or permanently charged lipid will typically bear an opposite net charge at physiological pH.

Lipophilic or hydrophobic antigens can be directly loaded into liposomes or lipid nanoparticles by addition to the lipid mixture prior to the mixing process (e.g., extrusion, ethanol injection, in-line mixing, microfluidics). The hydrophobicity of the cargo enables spontaneous entrapment within the lipid compartment of the nanoparticle as they are formed. This method can be applied to naturally lipophilic or hydrophobic cargos.

Water soluble or hydrophilic antigens can be loaded passively into liposomes that contain an aqueous core. The antigen is added directly to the aqueous buffer that is used to form the vesicles. Liposomes are made using established methods (e.g., extrusion, ethanol injection, in-line mixing, microfluidics, and the like). This method generally yields low entrapment, but the quantity of cargo can be controlled by the concentrations used. Small quantities of antigen are used for the induction of tolerance.

Examples of formulations for liposome vaccine systems are provided in the following: Schewendener 2014 Ther Adv Vaccines 2:159-182; Schmidt et al., 2016 Pharmaceutics 8:7; and Kersten 1995 Biochim Biophys Acta 1241:117-138.

In certain embodiments, the permanently charged or ionizable lipid of LNP or liposome delivery vehicles comprises a head group and a linear or branched lipophilic moiety, e.g. having the structure of Formula I described above.

In one embodiment, the permanently charged or ionizable lipid comprises a scaffold moiety. The scaffold moiety in one embodiment is represented by L (L1+L2+L3+L4+L5) of Formula I and at least one R is present as a hydrocarbon side chain, wherein n+p is 1 or 1 to 8 or 1 to 7, or 1 to 6 or 1 to 5 or 1 to 4 or 1 to 3.

L1 of Formula I may be linked to a head group directly or via a linker. The linker may be linear, branched or a ring structure. Examples of suitable linker groups for ionizable or permanently charged lipids are well known in the art as provided in WO 2021/026647 (PCT/CA2020/051098), which is incorporated herein by reference.

Examples of headgroups that may be linked directly or indirectly via a linker region to L1 include the following:
(i) ionizable cationic moieties selected from the group consisting of:

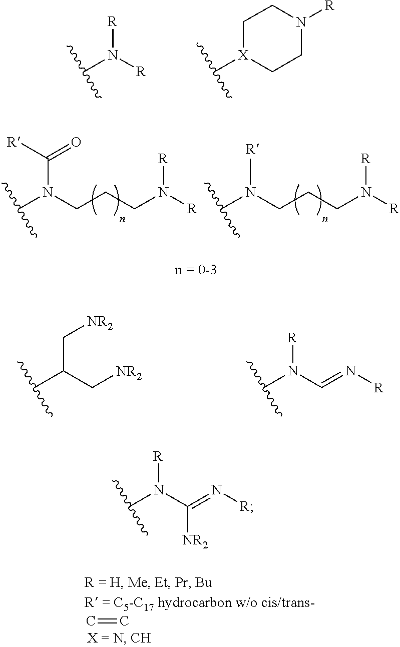

(ii) permanently charged moieties selected from the group consisting of:

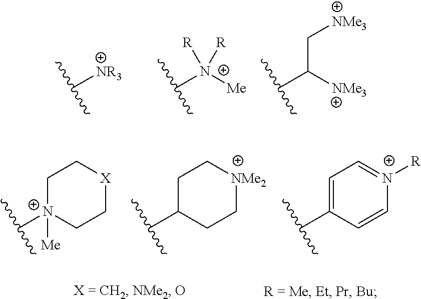

(iii) ionizable anionic moieties selected from the group consisting of:

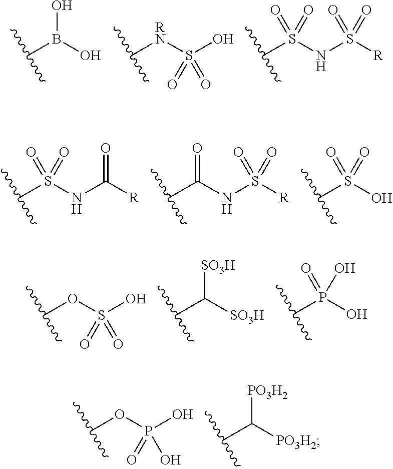

or
(iv) zwitterionic moieties selected from the group consisting of:

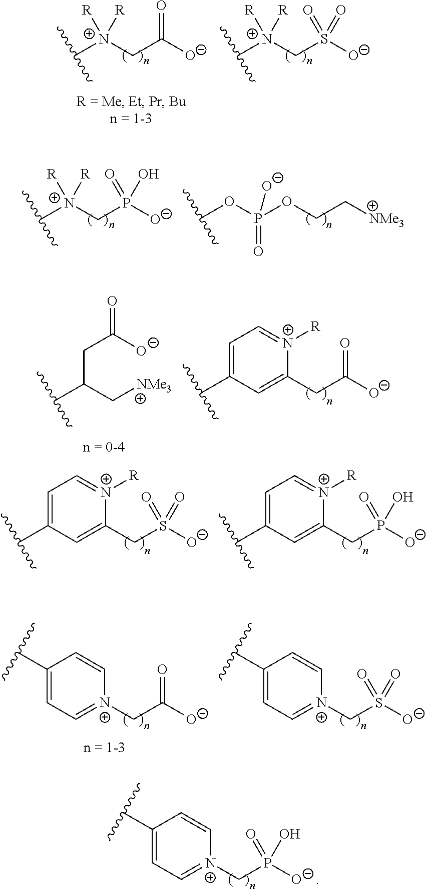

### Selective Delivery to Antigen Presenting Cells (APCs)

The compositions described herein may be used for delivery to APCs. By way of example and without being limiting, in vivo APC uptake of the delivery vehicles may be demonstrated in the pancreatic islets (e.g., where APCs interact with beta cells to pick up beta-cell antigens in the case of Type 1 Diabetes) or in the pancreatic lymph nodes (e.g., where APCs interact and with T cells to present antigens and instruct the type of T cell response that will be initiated to that antigen). In one embodiment, the uptake of the delivery vehicle is selective for APCs, with limited uptake in non-APC immune cells. Determination of whether uptake is selective for APCs may be carried out using the methods of Example 1 herein. For example, there may be limited delivery of the delivery vehicles to endocrine cells in the islets, and non-APC immune cells (e.g., T cells) in the lymph node.

For example, the total population of delivery vehicles, such as lipid nanoparticles, in a formulation described herein that are delivered to non-APCs may be less than 20%, less than 15% or less than 10% as measured using the techniques of Example 1.

In some embodiments, the delivery vehicles are lipid nanoparticles which preferentially deliver to antigen presenting cells (APCs) in pancreatic islets and/or lymph nodes.

### Methods of Administration

In some embodiments, the delivery vehicle comprising the lipid conjugate is part of a pharmaceutical formulation that is administered to treat, prevent and/or ameliorate an antigen-induced disorder or undesired antigen-driven immune response in a subject. The pharmaceutical formulation may be administered at any suitable dosage, and may comprise a pharmaceutically acceptable excipient.

In those embodiments in which the lipid conjugate(s) is formulated in a first delivery vehicle and the antigen or the nucleic acid that encodes the antigen is formulated in a second delivery vehicle, the first and second delivery vehicles may be administered separately or together. If administered separately, the first and second delivery vehicles may be administered sequentially to a subject. That is, the first delivery vehicle may be administered before the second or vice versa. The time frame between administration of the first and second delivery vehicles can be selected based on patient requirements. The first and second delivery vehicles are typically each part of a pharmaceutical formulations comprising suitable excipients and pharmaceutically acceptable salts.

In one embodiment, the pharmaceutical formulation or formulations are administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The compositions described herein may be administered to a subject, including a patient. This includes a human or a non-human subject. In some embodiments, the subject is human.

The subject selected for treatment may have a pathological condition associated with antigen-specific immune stimulation or undesired antigen-driven immune response. These immune disorders and undesired antigen-driven immune responses are diverse and range from allergic reactions to autoimmune diseases to transplant rejection. In some embodiments, the antigen-induced disorder is selected from: autoimmune diseases (T cell and/or antibody responses to self antigen), allergic diseases (T cell and IgE responses to environmental or food antigens), transplantation (T cell responses against major and minor histocompatibility antigens in donor tissue/organ/cell), anti-drug antibody responses (antibody responses that diminish efficacy of therapeutics), or gene/protein replacement therapy (T cell/antibody response against proteins therapeutically replaced in genetic protein deficiencies). In some embodiments, the antigen-induced disorder is selected from: multiple sclerosis, rheumatoid arthritis, myelin oligodendrocyte glycoprotein antibody disorder, vitiligo, type 1 diabetes, primary biliary cholangitis, anti-GBM nephritis/Goodpasture's disease, celiac disease, psoriasis, myasthenia gravis, immune thrombocytopenia purpura, Grave's disease, neuromyelitis optica, pemphigus vulgaris, bullous pemphigoid, cicatricial pemphigoid, lupus including systemic lupus erythematosus SLE, autoimmune liver disease, myositis, Evan's syndrome, transverse myelitis, Guillain-Barré syndrome, warm autoimmune hemolytic anemia, chronic inflammatory demyelinating polyneuropathy, autoimmune dysautonomia, autoimmune angioedema, Hashimoto's thyroiditis, Lambert-Eaton syndrome, peanut/legume allergy, tree nut allergy (antigens from any of cashew, pistachio, hazelnut, walnut, almond), egg allergy, cow's milk allergy, soy allergy, fish allergy, shellfish allergy, sesame allergy, wheat allergy, allergic airway disease, or allergies caused by environmental allergens (antigens from pollen, dust, pet dander, mold and cockroaches).

### Exemplary Embodiments

Various non-limiting embodiments disclosed herein are defined below:

Embodiment 1. An immunomodulatory combination comprising: a lipid conjugate comprising an immunomodulatory agent covalently linked to a lipophilic moiety by a cleavable linkage or through a cleavable linker; and an antigen and/or one or more nucleic acids that encode the antigen, wherein the antigen is a protein, polypeptide, peptide, lipoprotein, glycolipid, polynucleotide, or polysaccharide, wherein the lipid conjugate and the antigen and/or the one or more nucleic acids that encode the antigen are formulated in separate delivery vehicles or co-formulated in the same delivery vehicle.

Embodiment 2. The immunomodulatory combination of embodiment 1, wherein the delivery vehicles are lipid nanoparticles and/or liposomes, optionally wherein the delivery vehicles are lipid nanoparticles which effectively deliver to antigen presenting cells (APCs) in pancreatic islets and/or lymph nodes.

Embodiment 3. The immunomodulatory combination of embodiment 2, wherein: the antigen or the one or more nucleic acids encoding the antigen is entrapped within a lipid nanoparticle or liposome and has a net charge that is opposite a net charge of a lipid in the lipid nanoparticle, or the antigen is lipophilic and incorporated into a lipid compartment of a lipid nanoparticle or liposome, or wherein the antigen is hydrophilic and entrapped in a liposome containing an aqueous core.

Embodiment 4. The immunomodulatory combination of any one of embodiments 1 to 3, wherein the lipid conjugate is co-formulated in the same delivery vehicle as the antigen or the one or more nucleic acids that encode the antigen.

Embodiment 5. The immunomodulatory combination of any one of embodiment 1 to 4, wherein the lipid conjugate is a first lipid conjugate, and the immunomodulatory combination further comprises a second lipid conjugate, wherein the second lipid conjugate comprises an immunomodulatory agent covalently linked to a lipophilic moiety by a cleavable linkage or through a cleavable linker, wherein the immunomodulatory agent of the second lipid conjugate is different from the immunomodulatory agent of the first lipid conjugate, wherein the first lipid conjugate and the second lipid conjugate are formulated in separate delivery vehicles or are co-formulated in the same delivery vehicle, optionally wherein the immunomodulatory combination comprises a plurality of lipid conjugates, wherein the plurality of lipid conjugates comprise 3, 4, 5, 6, 7, 8, 9, or 10 immunomodulatory agents, wherein each immunomodulatory agent of the plurality of lipid conjugates is different, and wherein each lipid conjugate of the plurality of lipid conjugates is independently formulated in a separate delivery vehicle from the other components of the immunomodulatory combination or is co-formulated with one or more of the other components of the immunomodulatory combination.

Embodiment 6. The immunomodulatory combination of embodiment 5, wherein the immunomodulatory agent of the second lipid conjugate targets a different immune pathway than the immunomodulatory agent of the first lipid conjugate.

Embodiment 7. The immunomodulatory combination of embodiment 5 or 6, wherein the first lipid conjugate and the second lipid conjugate are co-formulated in the same delivery vehicle.

Embodiment 8. The immunomodulatory combination of any one of embodiments 1 to 7, wherein each immunomodulatory agent is a tolerogenic agent or an anti-inflammatory agent.

Embodiment 9. The immunomodulatory combination of any one of embodiments 1 to 7, wherein each immunomodulatory agent is an immunostimulant or an immunosuppressant.

Embodiment 10. The immunomodulatory combination of any one of embodiments 1 to 9, wherein each immunomodulatory agent is independently: a non-steroidal anti-inflammatory drug (NSAIDs), an inflammasome inhibitor, a Janus kinase (JAK) inhibitor, a corticosteroid, an mTOR inhibitor, a DMARD (disease-modifying antirheumatic drug), a calcineurin inhibitor, or a vitamin D receptor agonist.

Embodiment 11. The immunomodulatory combination of any one of embodiments 1 to 9, wherein each immunomodulatory agent is independently prednisone, budesonide, prednisolone, methylprednisolone, hydrocortisone, cortisone, betamethasone, budesonide, triamcinolone, flunisolide, beclomethasone, fluticasone, mometasone, fludrocortisone, flumethasone, triamcinolone acetonide, isoflupredone, corticosterone, desoxycortone acetate, desoxycortone enanthate, 11-deoxycorticosterone, 11-deoxycortisol, aldosterone, dexamethasone, calcitriol, acetylsalicylic acid, salicylate, mycophenolic acid, sirolimus, tacrolimus, cholecalciferol, calcifediol, alfacalcidol, calcipotriol, falecalcitriol, maxacalcitol, paricalcitol, doxercalciferol, 22-oxacalcitriol, tacalcitol, eldecalcitol, elocalcitol, inecalcitol, becocalcidiol, seocalcital, ergocalciferol, lexacalcitol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, or Ethyl 2-((2-chlorophenyl) (hydroxy)methyl) acrylate.

Embodiment 12. The immunomodulatory combination of any one of embodiments 1 to 11, wherein the antigen is a first antigen, and the immunomodulatory combination further comprises a second antigen or one or more nucleic acids encoding the second antigen, wherein the first antigen is different than the second antigen, wherein the second antigen or the one or more nucleic acids encoding the second antigen is formulated in a separate delivery vehicle from the other components of the immunomodulatory combination or is co-formulated with one or more of the other components of the immunomodulatory combination, optionally wherein the immunomodulatory combination comprises a plurality of antigens or comprises one or more nucleic acids encoding the plurality of antigens, or comprises a combination of antigens and antigen-encoding nucleic acid(s) for providing the plurality of antigens, wherein the plurality of antigens comprises 3, 4, 5, 6, 7, 8, 9, or 10 antigens, and each antigen or antigen-encoding nucleic acid is independently formulated in a separate delivery vehicle from the other components of the immunomodulatory combination or is co-formulated with one or more of the other components of the immunomodulatory combination.

Embodiment 13. The immunomodulatory combination of embodiment 12, wherein the one or more nucleic acids encoding the first antigen and the one or more nucleic acids encoding the second antigen are comprised within a single nucleic acid and the first antigen and the second antigen are co-formulated in the same delivery vehicle.

Embodiment 14. The immunomodulatory combination of any one of embodiments 1 to 13, for use in treatment of a subject having an antigen-induced disorder or undesired antigen-driven immune response, or for use in manufacture of a medicament for treating the subject, optionally wherein the antigen-induced disorder or undesired antigen-driven immune response is selected from: autoimmune diseases (T cell and/or antibody responses to self antigen), allergic diseases (T cell and IgE responses to environmental or food antigens), transplantation (T cell responses against major and minor histocompatibility antigens in donor tissue/organ/cell), anti-drug antibody responses (antibody responses that diminish efficacy of therapeutics), gene/protein replacement therapy (T cell/antibody response against proteins therapeutically replaced in genetic protein deficiencies), optionally wherein the antigen-induced disorder or undesired antigen-driven immune response is selected from: multiple sclerosis, rheumatoid arthritis, myelin oligodendrocyte glycoprotein antibody disorder, vitiligo, type 1 diabetes, primary biliary cholangitis, anti-GBM nephritis/Goodpasture's disease, celiac disease, psoriasis, myasthenia gravis, immune thrombocytopenia purpura, Grave's disease, neuromyelitis optica, pemphigus vulgaris, bullous pemphigoid, cicatricial pemphigoid, lupus including systemic lupus erythematosus SLE, autoimmune liver disease, myositis, Evan's syndrome, transverse myelitis, Guillain-Barré syndrome, warm autoimmune hemolytic anemia, chronic inflammatory demyelinating polyneuropathy, autoimmune dysautonomia, autoimmune angioedema, Hashimoto's thyroiditis, Lambert-Eaton syndrome, peanut/legume allergy, tree nut allergy (antigens from any of cashew, pistachio, hazelnut, walnut, almond), egg allergy, cow's milk allergy, soy allergy, fish allergy, shellfish allergy, sesame allergy, wheat allergy, allergic airway disease, and allergies caused by environmental allergens (antigens from pollen, dust, pet dander, mold and cockroaches).

Embodiment 15. A method for treating a subject having an antigen-induced disorder or undesired antigen-driven immune response comprising administering the immunomodulatory combination of any one of embodiments 1 to 13 to the subject, wherein the antigen, or the one or more nucleic acids encoding the antigen, and the lipid conjugate are administered together or sequentially, optionally wherein the antigen-induced disorder or undesired antigen-driven immune response is selected from: autoimmune diseases (T cell and/or antibody responses to self antigen), allergic diseases (T cell and IgE responses to environmental or food antigens), transplantation (T cell responses against major and minor histocompatibility antigens in donor tissue/organ/cell), anti-drug antibody responses (antibody responses that diminish efficacy of therapeutics), gene/protein replacement therapy (T cell/antibody response against proteins therapeutically replaced in genetic protein deficiencies), optionally wherein the antigen-induced disorder or undesired antigen-driven immune response is selected from: multiple sclerosis, rheumatoid arthritis, myelin oligodendrocyte glycoprotein antibody disorder, vitiligo, type 1 diabetes, primary biliary cholangitis, anti-GBM nephritis/Goodpasture's disease, celiac disease, psoriasis, myasthenia gravis, immune thrombocytopenia purpura, Grave's disease, neuromyelitis optica, pemphigus vulgaris, bullous pemphigoid, cicatricial pemphigoid, lupus including systemic lupus erythematosus SLE, autoimmune liver disease, myositis, Evan's syndrome, transverse myelitis, Guillain-Barré syndrome, warm autoimmune hemolytic anemia, chronic inflammatory demyelinating polyneuropathy, autoimmune dysautonomia, autoimmune angioedema, Hashimoto's thyroiditis, Lambert-Eaton syndrome, peanut/legume allergy, tree nut allergy (antigens from any of cashew, pistachio, hazelnut, walnut, almond), egg allergy, cow's milk allergy, soy allergy, fish allergy, shellfish allergy, sesame allergy, wheat allergy, allergic airway disease, and allergies caused by environmental allergens (antigens from pollen, dust, pet dander, mold and cockroaches).

Embodiment 16. A vaccine formulation comprising: at least one prodrug comprising an immunomodulatory agent that is conjugated to a lipid moiety; and at least one antigen and/or a nucleic acid that encodes the antigen, wherein the at least one antigen is a protein, polypeptide, peptide, lipoprotein, glycolipid, polynucleotide, or polysaccharide, wherein the at least one prodrug and the antigen and/or the nucleic acid that encodes the antigen are formulated in separate delivery vehicles or co-formulated in the same delivery vehicles in the formulation.

Embodiment 17. The vaccine formulation of embodiment 16, wherein the delivery vehicles are lipid nanoparticles.

Embodiment 18. The vaccine formulation of embodiment 16 or 17, wherein the at least one prodrug is co-formulated in the same delivery vehicles with the antigen or the nucleic acid that encodes the antigen.

Embodiment 19. The vaccine formulation of embodiment 16, 17, or 18, wherein two prodrugs are present in the formulation and are formulated separately or co-formulated in the same delivery vehicle.

Embodiment 20. The vaccine formulation of any one of embodiments 16 to 19, wherein the immunomodulatory agent is a tolerogenic agent.

Embodiment 21. The vaccine formulation of any one of embodiments 16 to 19, wherein the immunomodulatory agent is an immunostimulant or an immunosuppressant.

Embodiment 22. The vaccine formulation of any one of embodiments 16 to 21, wherein the immunomodulatory agent of the at least one prodrug is selected from dexamethasone, calcitriol, acetylsalicylic acid, mycophenolic acid, sirolimus, tacrolimus, cholecalciferol, calcifediol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, Ethyl 2-((2-chlorophenyl) (hydroxy)methyl) acrylate, or combinations thereof.

Embodiment 23. The vaccine formulation of any one of embodiments 16 to 22, wherein two antigens and/or nucleic acid encoding the antigens are present in the formulation and are formulated separately or co-formulated in the same delivery vehicles.

Embodiment 24. A method for treating a subject having an antigen-induced disorder comprising: administering at least one prodrug comprising an immunomodulatory agent that is conjugated to a lipophilic moiety; and administering at least one antigen and/or a nucleic acid that encodes the antigen, wherein the at least one antigen is a protein, polypeptide, peptide, lipoprotein, glycolipid, polynucleotide, or polysaccharide, wherein the at least one prodrug and the antigen and/or the nucleic acid that encodes the antigen are formulated separately in delivery vehicles or co-formulated in the same delivery vehicle, and wherein the at least one prodrug and the at least one antigen and/or nucleic acid that encodes the antigen are administered together or sequentially.

Embodiment 25. The method of embodiment 24, wherein the delivery vehicle is a lipid nanoparticle.

Embodiment 26. The method of embodiment 24 or 25, wherein the at least one prodrug is co-formulated in the delivery vehicle with the antigen and/or the nucleic acid that encodes the antigen.

Embodiment 27. The method of embodiment 24, 25, or 26, wherein two prodrugs are present in the formulation and are formulated separately or co-formulated in the same delivery vehicle.

Embodiment 28. The method of any one of embodiments 24 to 27, wherein the immunomodulatory agent is a tolerogenic agent.

Embodiment 29. The method of any one of embodiments 24 to 28, wherein the immunomodulatory agent is an immunostimulant or an immunosuppressant.

Embodiment 30. The method of any one of embodiments 24 to 29, wherein the immunomodulatory agent of the at least one prodrug is selected from dexamethasone, calcitriol, acetylsalicylic acid, mycophenolic acid, sirolimus, tacrolimus, cholecalciferol, calcifediol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, Ethyl 2-((2-chlorophenyl) (hydroxy)methyl) acrylate, or combinations thereof.

Embodiment 31. The method of any one of embodiments 24 to 30, wherein two antigens and/or nucleic acid encoding the antigens are present in the formulation and are formulated separately or co-formulated in the delivery vehicles.

Embodiment 32. Use of a prodrug comprising an immunomodulatory agent that is conjugated to a lipophilic moiety to treat a subject having an antigen-induced disorder in combination with at least one antigen and/or a nucleic acid that encodes the at least one antigen, wherein the at least one antigen is a protein, polypeptide, peptide, lipoprotein, glycolipid, polynucleotide, or polysaccharide, and wherein the at least one prodrug and the antigen and/or the nucleic acid that encodes the antigen are (i) co-formulated in the same delivery vehicles in an at least one vaccine formulation, or (ii) formulated in separate delivery vehicles for sequential administration or co-administration to the subject.

Embodiment 33. The use of embodiments 32, wherein the delivery vehicles are lipid nanoparticles.

Embodiment 34. The use of embodiments 32 or 33, wherein the at least one prodrug is co-formulated in the same delivery vehicles with the antigen or the nucleic acid that encodes such antigen.

Embodiment 35. The use of embodiments 32, 33 or 34, wherein two prodrugs are present in the formulation and are formulated separately or co-formulated in the same delivery vehicles.

Embodiment 36. The use of any one of embodiments 32 to 35, wherein the immunomodulatory agent is a tolerogenic agent.

Embodiment 37. The use of any one of embodiments 32 to 35, wherein the immunomodulatory agent is an immunostimulant or an immunosuppressant.

Embodiment 38. The use of any one of embodiments 32 to 35, wherein the immunomodulatory agent of the at least one prodrug is selected from dexamethasone, calcitriol, acetylsalicylic acid, mycophenolic acid, sirolimus, tacrolimus, cholecalciferol, calcifediol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, Ethyl 2-((2-chlorophenyl) (hydroxy)methyl) acrylate, or combinations thereof.

Embodiment 39. The use of any one of embodiments 32 to 38, wherein two antigens and/or nucleic acid encoding the antigens are present in the formulation and are formulated separately or co-formulated in the delivery vehicles.

Embodiment 40. A combination of a prodrug comprising an immunomodulatory agent that is conjugated to a lipophilic moiety and at least one antigen and/or a nucleic acid that encodes the at least one antigen to treat a subject having an antigen-induced disorder, wherein the at least one antigen is a protein, polypeptide, peptide, lipoprotein, glycolipid, polynucleotide, or polysaccharide, and wherein the at least one prodrug and the antigen and/or the nucleic acid that encodes the antigen are (i) co-formulated in a delivery vehicle, or (ii) formulated in separate delivery vehicles for sequential administration or co-administration to the subject.

The following examples are given for the purpose of illustration only and not by way of limitation on the scope of the invention.

### EXAMPLES

### Synthesis of Lipid Conjugates

Various lipid conjugates were prepared using the synthesis procedures A-E set forth below.

All reagents and solvents were purchased from commercial suppliers and used without further purification unless otherwise stated, except THF, (freshly distilled from Na/benzophenone under nitrogen), and Et₃N, DMF and CH₂Cl₂ (freshly distilled from CaH₂ under nitrogen). USP grade castor oil was purchased at a local pharmacy (Life™ Brand) and used as received. For NMR, chemical shifts are reported in parts per million (ppm) on the δ scale and coupling constants, J, are in hertz (Hz). Multiplicities are reported as “s” (singlet), “d” (doublet), “dd” (doublet of doublets), “dt” (doublet of triplets), “ddd” (doublet of doublets of doublets), “t” (triplet), “td” (triplet of doublets), “q” (quartet), “quin” (quintuplet), “sex” (sextet), “m” (multiplet), and further qualified as “app” (apparent) and “br” (broad).

The steps of the general synthesis of lipid conjugates based on hydroxy and carboxy derivatives of castor oil (ricinolein) are provided below in Scheme 1. This is followed by Scheme 1, referred to as general procedures A-E, describing the steps for producing the lipid conjugates of Examples E, S, T, and W below.

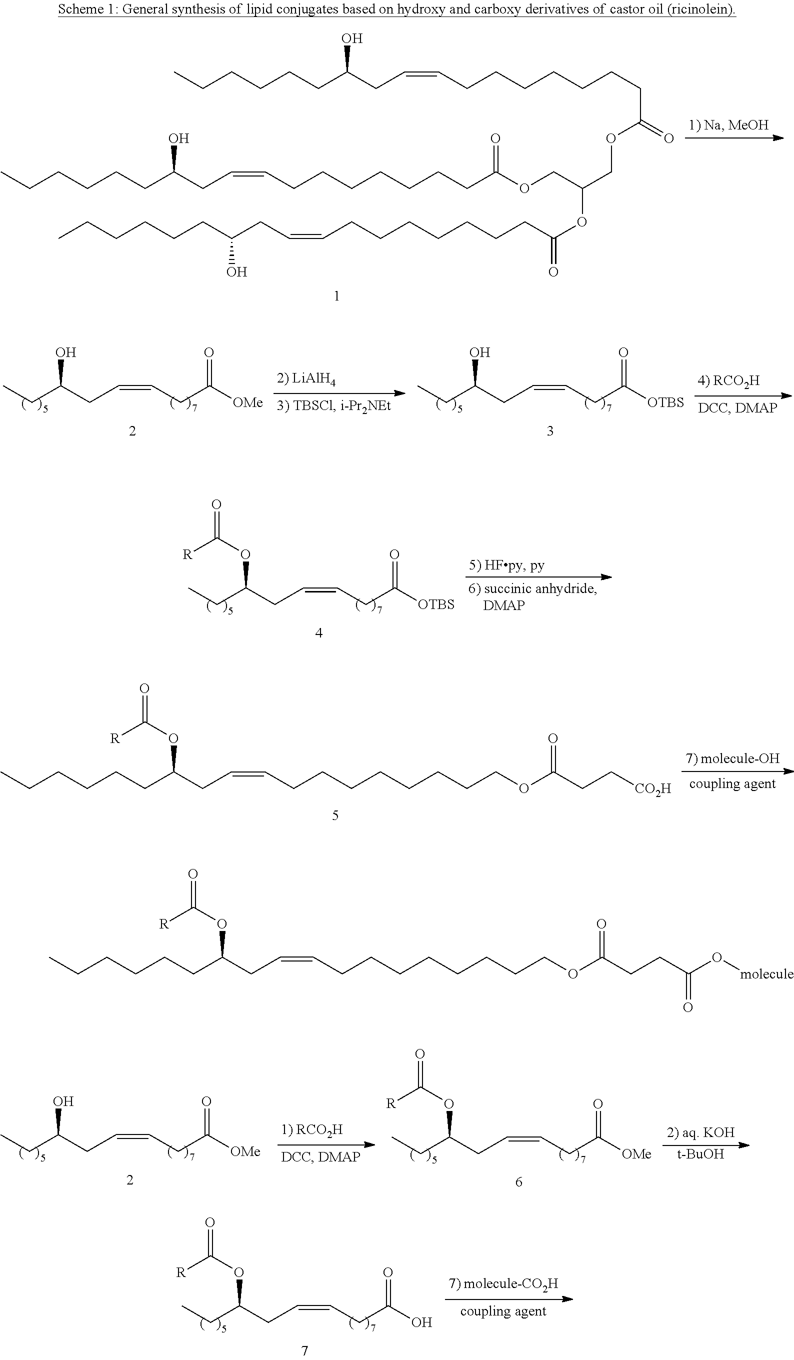

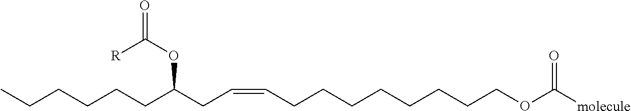

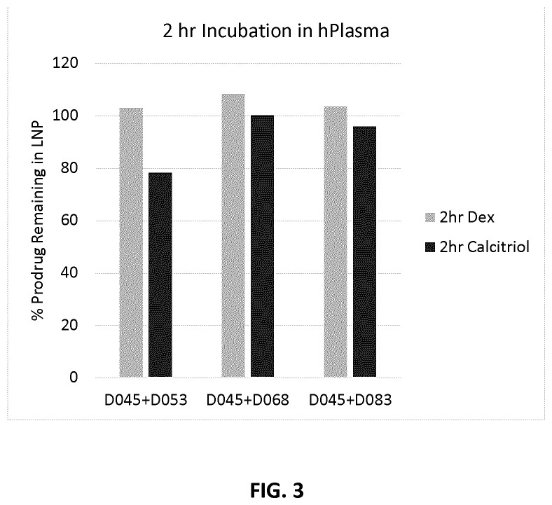

According to the synthesis reaction described above in Scheme I, castor oil, also known as ricinolein (a glyceride of ricinoleic acid) is the starting material for the synthesis of the pro-drugs shown in FIG. 3.

In step 1) above, sodium methoxide (2.0 mL of 3.0 M solution in MeOH, 6.00 mmol, 0.20 equiv.) was added to a stirring, room temperature 1:1 THF/MeOH (30 mL) solution of the castor oil (28.0 g, 30.0 mmol, 1.00 equiv.) in a round bottom flask under argon. After 14 h, the reaction mixture was quenched with saturated aqueous NH₄Cl and extracted with Et₂O (3×150 mL). The combined organic layers were washed with water (1×150 mL), brine (1×150 mL), dried over Na₂SO₄ and concentrated to produce a clear, colourless oil of methyl (12R)-hydroxyoleate 1 (28.0 g, quantitative yield), which was used without further purification. The structure of methyl (12R)-hydroxyoleate and its physical properties are shown below:

### Methyl (12R)-hydroxyoleate (1)

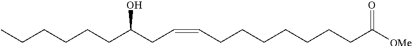 [SMILES: CCCCCC[C@@H](O)C/C=C\CCCCCCCC(=O)OC]

R_{f}=0.50 (SiO₂, 70:30 hexanes/EtOAc);

¹H NMR (300 MHz, CDCl₃): δ 5.64-5.50 (m, 1H), 5.49-5.35 (m, 1H), 3.68 (s, 3H), 3.63 (quint., J=5.6 Hz, 1H), 2.32 (t, J=7.6 Hz, 2H), 2.23 (t, J=6.6 Hz, 2H), 2.13-2.00 (m, 2H), 1.72-1.19 (m, 20H), 0.90 (t, J=6.4 Hz, 3H).

According to 2) in the reaction scheme above, a room temperature THF (15 mL) solution of methyl (12R)-hydroxyoleate (9.37 g, 30.0 mmol) was added from an addition funnel over 20-30 min to a stirred, ice-cold THF (90 mL) suspension of LiAlH₄ (1.25 g, 33.0 mmol, 1.10 equiv.) in a round bottom flask under argon. After the addition was complete, the cold bath was removed. After 14 h, the reaction mixture was cooled in an ice bath, diluted with Et₂O (150 mL) and quenched with a quenching solution (1.25 mL H₂O, 1.25 mL aqueous 1 M NaOH, 3.75 mL H₂O), stirred for 1 h at room temperature and filtered through Celite, while washing thoroughly with Et₂O. The filtrate was concentrated on a rotary evaporator to yield the crude diol as a pale yellow oil (quantitative yield), which was used without further purification.

According to 3) of the above reaction scheme, a room temperature DMF (20 mL) solution of tert-butyldimethylsilyl chloride (3.96 g, 26.2 mmol, 1.00 equiv.) was added from an addition funnel over 30 min to a 10-15° C. DMF (25 mL) solution of the above diol (8.21 g, 28.9 mmol, 1.10 equiv.) and i-Pr₂Net (5.73 mL, 32.8 mmol, 1.25 equiv.) in a round bottom flask under argon. The reaction mixture was allowed to warm up over 14 h, then quenched with saturated aqueous NH₄Cl and extracted with 1:1 Et₂O/hexanes (3×100 mL). The combined organic layers were washed with H₂O (3×100 mL), brine (1×100 mL), dried over Na₂SO₄ and concentrated on a rotary evaporator to produce the crude primary silyl ether as a pale yellow oil. The crude was purified by filtration through a plug of silica gel (220 mL SiO₂, 99:1→95:5 hexanes/EtOAc) to yield a clear, colourless oil composed of the silyl ether 2 (8.38 g, 80% yield). The structure of the silyl ether 2 is shown below, as well as its physical properties:

### I-1-(tert-Butyldimethylsilyl)-12-hydroxyoleyl alcohol (2)

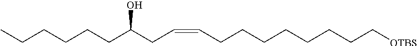 [SMILES: CCCCCC[C@@H](O)C/C=C\CCCCCCCCO[Si](C)(C)C(C)(C)C]

R_{f}=0.16 (SiO₂, 95:5 hexanes/EtOAc);

¹H NMR (300 MHZ, CDCl₃): δ 5.64-5.50 (m, 1H), 5.49-5.35 (m, 1H), 3.68 (s, 3H), 3.63 (quint., J=5.6 Hz, 1H), 2.32 (t, J=7.6 Hz, 2H), 2.23 (t, J=6.6 Hz, 2H), 2.13-2.00 (m, 2H), 1.72-1.19 (m, 20H), 0.90 (t, J=6.4 Hz, 3H).

According to 4) of the above reaction scheme, N,N′-Dicyclohexylcarbodiimide (DCC) (495 mg, 2.40 mmol, 1.20 equiv.) was added to an ice-cold CH₂Cl₂ (6 mL) solution of RCO₂H (279 mg, 2.40 mmol, 1.20 equiv.) in a round bottom flask under argon, and the ice bath was subsequently removed and the resultant mixture stirred for 15 min. In this example, RCO₂H was hexanoic acid, although other acyl groups can be utilized to produce a desired hydrocarbon side chain S. The reaction mixture was cooled again in an ice bath, a CH₂Cl₂ (2 mL) solution of the silyl ether, I-1-(tert-Butyldimethylsilyl)-12-hydroxyoleyl alcohol 2 (797 mg, 2.00 mmol) was added, followed by DMAP (366 mg, 3.00 mmol, 1.50 equiv.), and the reaction mixture was allowed to warm to room temperature over 14 h. The reaction mixture was diluted with Et₂O, stirred for 10 min, then filtered through Celite. The filtrate was concentrated on a rotary evaporator to yield the crude ester as a white semi-solid. The crude was purified by filtration through a plug of silica gel (20 mL SiO₂, 95:5 hexanes/EtOAc) to produce a clear, colourless oil as the intermediate ester (quantitative yield) having an R_{f}=0.53 (SiO₂, 90:10 hexanes/EtOAc).

According to 5) of the reaction scheme above, neat HF·pyridine solution (0.74 mL of 70% HF in pyridine, 6.00 mmol, 3.00 equiv.) was added to a stirred, ice-cold THF (6 mL) solution of pyridine (0.48 mL, 6.00 mmol, 3.00 equiv.) and the above silyl ether (2.00 mmol) in a round bottom flask under argon. After 2 h, the reaction mixture was quenched with saturated aqueous NaHCO₃. The mixture was extracted with Et₂O (2×10 mL), then the combined organic extracts were washed with H₂O (1×10 mL), brine, dried over Na₂SO₄ and concentrated on a rotary evaporator to afford the crude primary alcohol. The crude was purified by filtration through a plug of silica gel (20 mL, 90:10 hexanes/EtOAc) to produce a primary alcohol 3 (quantitative yield) as a clear, colourless oil having the structure and physical properties below:

### (12R)-Hexanoyloxyoleyl Alcohol (3)

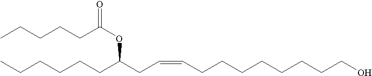 [SMILES: CCCCCC[C@H](C/C=C\CCCCCCCCO)OC(=O)CCCCC]

According to 6) of the reaction scheme above, solid succinic anhydride (400 mg, 4.00 mmol, 2.00 equiv.) and DMAP (611 mg, 5.00 mmol, 2.50 equiv.) were added to a stirring room temperature CH₂Cl₂ (6 mL) solution of the (12R)-Hexanoyloxyoleyl alcohol (3) (765 mg, 2.00 mmol, 1.00 equiv.) in a round bottom flask under argon. After 14 hours, the reaction was quenched with aqueous 1 M HCl and extracted with CH₂Cl₂ (2×15 mL). The combined organic extracts were then washed with aqueous 1 M HCl (1×15 mL), H₂O (2×15 mL), dried over Na₂SO₄ and concentrated on a rotary evaporator to afford the intermediate hemisuccinate (quantitative yield) as a pale yellow oil that was used without further purification. The intermediate had an R_{f}=0.32 (SiO₂, 50:50 hexanes/EtOAc).

According to 7) in the reaction scheme, solid DCC (99 mg, 0.48 mmol, 1.20 equiv.) was added to a stirring, ice-cold CH₂Cl₂ (2 mL) solution of the above hemisuccinate (232 mg, 0.48 mmol, 1.20 equiv.) in a round bottom flask under argon, then the ice bath was removed and the resultant mixture stirred for 15 min. The reaction mixture was cooled again in an ice bath and solid dexamethasone (157 mg, 0.40 mmol) and DMAP (73 mg, 0.60 mmol, 1.50 equiv.) were added. The reaction mixture was allowed to warm up over 14 h, diluted with Et₂O, stirred for 10 min, then filtered through Celite. The filtrate was concentrated to produce the crude, which was a pale yellow oil. The crude was purified by flash column chromatography (50 mL SiO₂, 80:20→50:50 hexanes/EtOAc) to yield a clear, colourless oil as desired pro-drug 4 (328 mg, 95% yield) having the structure and properties below:

### 2-((8S,9R,10S,11S,13S,14S,16R,17R)-9-Fluoro-11,17-dihydroxy-10,13,16-trimethyl-3-oxo-6,7,8,9,10,11,12,13,14,15,16,17-dodecahydro-3H-cyclopenta[a]49ctadic49rene-17-yl)-2-oxoethyl ((R,Z)-12-(hexanoyloxy) 49ctadic-9-en-1-yl) succinate (4)

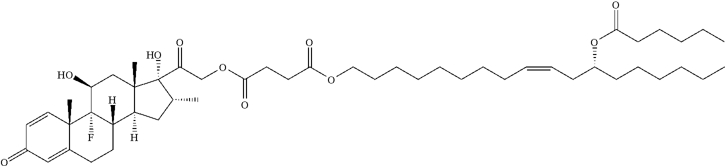

R_{f}=0.38 (SiO₂, 50:50 hexanes/EtOAc);

¹H NMR (300 MHz, CDCl₃): δ 7.22 (dd, J=10.2, 3.9, 1H), 6.32 (dd, J=10.2, 1.7, 1H), 6.1 (s, 1H), 5.44-5.17 (m, 9H), 5.00-4.81 (m, 2H), 4.43-4.22 (m, 4H), 4.21-4.06 (m, 2H), 3.16-3.01 (m, 1H), 2.84-2.51 (m, 11H), 2.50-2.23 (m, 9H), 2.21-1.48 (m, 25H), 1.45-1.15 (m, 34H), 1.14-1.00 (m, 1H), 1.03 (s, 3H), 0.95-0.81 (m, 10H).

The lipid conjugate is based on a ricinoleyl scaffold L with a hexanoyl (C6:0) side chain conjugated to dexamethasone by a succinate linker (INT-D034).

In the above example, RCO₂H added in 4) of the above reaction was hexanoic acid to produce the hexanoyl side chain (C6:0), although other fatty acids can be utilized to produce a desired hydrocarbon side chain R on the ricinoleyl scaffold.

### General Procedure A—Acylation of (R)-1-(tert-Butyldimethylsilyl)-12-hydroxyoleyl Alcohol 3 (4a-h)

DCC (1.20 equiv.) was added to a stirring, ice-cold CH₂Cl₂ solution of the desired carboxylic acid (1.20 equiv.) in a round bottom flask under argon, then the ice bath was removed and the resultant stirred for 15 min. The reaction mixture was cooled again in an ice bath, a CH₂Cl₂ solution of alcohol 3 (1.00 equiv., 0.25 M in CH₂Cl₂) was added, followed by DMAP (1.50 equiv.), and the reaction mixture was allowed to warm to room temperature over 14 h. The reaction mixture was diluted with Et₂O, stirred for 10 min, then filtered through Celite®. The filtrate was concentrated on a rotary evaporator to yield the crude ester as a white semi-solid. The crude was purified by filtration through a plug of silica gel (95:5 hexanes/EtOAc) to afford the pure ester.

### General Procedure B—Desilylation-Succinylation of (12R)-Acyloxyoleyl Alcohols 4a-h (5a-h)

HF·pyridine solution (3.00 equiv. of 70% HF in pyridine) was added to a stirring, ice-cold THF (0.30 M relative to starting silyl ether) solution of pyridine (3.00 equiv.) and 12-acyl ricinoleyl alcohol silyl ether (1.00 equiv.) in a round bottom flask under argon. When TLC indicated consumption of the starting material (2-8 h), the reaction mixture was quenched with saturated aqueous NaHCO₃. The mixture was extracted with Et₂O (2×10 mL), then the combined organic extracts were washed with H₂O (1×10 mL), brine, dried over Na₂SO₄ and concentrated on a rotary evaporator to afford the crude primary alcohol. The crude was purified by filtration through a plug of silica gel (90:10 hexanes/EtOAc), concentrated on a rotary evaporator and dried under high vacuum to afford the primary alcohol as a clear, colourless oil and used in the subsequent succinylation without further purification.

Solid succinic anhydride (2.00 equiv.) and DMAP (2.50 equiv.) were added to a stirring, room temperature CH₂Cl₂ (0.30 M relative to starting primary alcohol) solution of 12-acyl ricinoleyl alcohol (1.00 equiv.) in a round bottom flask under argon. After 14 hours, the reaction was quenched with aqueous 1 M HCl and extracted with CH₂Cl₂ (2×15 mL). The combined organic extracts were then washed with aqueous 1 M HCl (1×15 mL), H₂O (2×15 mL), dried over Na₂SO₄ and concentrated on a rotary evaporator. The residue was redissolved in hexanes, treated with activated carbon, filtered through Celite® and the filtrate concentrated to afford the intermediate hemisuccinate as a colourless to pale yellow oil that was used without further purification.

### General Procedure C—Acylation of Methyl (12R)-Ricinoleate 2 (6a-c)

DCC (1.20 equiv.) was added to a stirring, ice-cold CH₂Cl₂ solution of the desired carboxylic acid (1.20 equiv.) in a round bottom flask under argon, then the ice bath was removed and the resultant stirred for 15 min. The reaction mixture was cooled again in an ice bath, a CH₂Cl₂ solution of methyl (12R)-ricinoleate (1.00 equiv., 0.30 M in CH₂Cl₂) was added, followed by DMAP (1.50 equiv.), and the reaction mixture was allowed to warm to room temperature over 14 h. The reaction mixture was diluted with hexanes, stirred for 10 min, then filtered through Celite®. The filtrate was concentrated on a rotary evaporator to yield the crude diester as a white semi-solid, which was purified by filtration through a plug of silica gel (95:5 hexanes/EtOAc) to afford the pure ester.

### General Procedure D—Conjugation of Dexamethasone to Hemisuccinates 5a-h

DCC (1.20 equiv.) was added to a stirring, ice-cold CH₂Cl₂ (0.2 M in dexamethasone) solution of 12-acyl ricinoleyl hemisuccinate (1.20 equiv.) in a round bottom flask under argon, then the ice bath was removed and the resultant stirred for 15 min. The reaction mixture was cooled again in an ice bath and solid dexamethasone (1.00 equiv.) and DMAP (1.50 equiv.) were added. The reaction mixture was allowed to warm up over 14 h, diluted with Et₂O, stirred for 10 min, then filtered through Celite®. The filtrate was concentrated to afford the crude as a pale yellow oil and subsequently purified by flash column chromatography (SiO₂. 80:20→50:50 hexanes/EtOAc) to afford a clear, colourless oil as the desired dexamethasone conjugate.

### General Procedure E—Conjugation of Dexamethasone to Ricinoleic Acids 12a-b, 13

DCC (1.10 equiv.) was added to a stirring, ice-cold CH₂Cl₂ (0.1 M in dexamethasone) solution of the acyloxystearic acid (1.10 equiv.) in a round bottom flask under argon, then the ice bath was removed and the resultant stirred for 15 min. The reaction mixture was cooled again in an ice bath and solid dexamethasone (1.00 equiv.) and DMAP (1.50 equiv.) were added. The reaction mixture was allowed to warm up over 14 h, diluted with Et₂O, stirred for 10 min, then filtered through Celite®. The filtrate was concentrated to afford the crude as a pale yellow oil and subsequently purified by flash column chromatography to afford a clear, colourless oil as the desired conjugate.

### (R,Z)-18-((tert-Butyldimethylsilyl)oxy) octadec-9-en-7-yl acetate (4a)

Acetyl chloride (0.43 mL, 6.00 mmol, 1.20 equiv.) was added dropwise to a stirring ice-cold CH₂Cl₂ (10 mL) solution of silyl ether 3 (2.00 g, 5.00 mmol, 1.00 equiv.), acetyl chloride (0.43 mL, 6.00 mmol, 1.20 equiv.), triethylamine (0.83 mL, 6.00 mmol, 1.2 equiv.) and DMAP (733 mg, 6.00 mmol, 1.20 equiv.) in a round bottom flask under argon, which was allowed to warm to room temperature. After 14 h, the reaction mixture was diluted with CH₂Cl₂, washed with saturated aqueous NH₄Cl (1×15 mL), water (2×15 mL) and dried over Na₂SO₄ and concentrated on a rotary evaporator. The residue was redissolved in eluent and passed through a plug of silica gel (30 mL SiO₂, 97:3 hexanes/EtOAc) to afford ester 4a (1.83 g, 83%) as a pale yellow oil.

R_{f}=0.45 (SiO₂, 95:5 hexanes/EtOAc);

¹H NMR (300 MHz, CDCl₃): δ 5.65-5.53 (m, 1H), 5.49-5.36 (m, 1H), 3.70-3.56 (m, 3H), 2.23 (t, J=6.8 Hz, 2H), 2.13-2.00 (m, 2H), 1.58-1.23 (m, 22H), 0.97-0.86 (m, 12H), 0.07 (s, 6H).

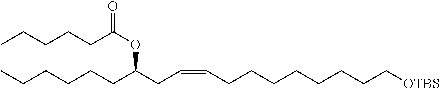 [SMILES: CCCCCC[C@H](C/C=C\CCCCCCCCO[Si](C)(C)C(C)(C)C)OC(=O)CCCCC]

### (R,Z)-18-((tert-Butyldimethylsilyl)oxy) octadec-9-en-7-yl hexanoate (4b)

According to General Procedure A, silyl ether 3 (2.00 g, 5.00 mmol), hexanoic acid (697 mg, 6.00 mmol), DCC (1.24 g, 6.00 mmol) and DMAP (916 mg, 7.50 mmol) in CH₂Cl₂ (15 mL) provided 2.37 g of ester 4b (2.39 g, quantitative yield) as a clear, colourless oil.

R_{f}=0.43 (SiO₂, 95:5 hexanes/EtOAc);

¹H NMR (300 MHz, CDCl₃): δ 5.56-5.42 (m, 1H), 5.41-5.27 (m, 1H), 4.90 (quint., J=6.3 Hz, 1H), 3.61 (t, J=6.6 Hz, 2H), 2.37-2.22 (m, 4H), 2.10-1.96 (m, 2H), 1.71-1.45 (m, 6H), 1.43-1.19 (m, 22H), 0.91 (br s, 15H), 0.07 (s, 6H).

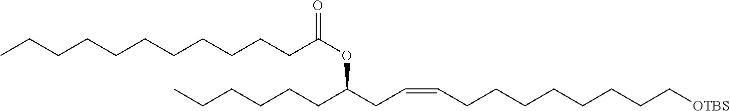 [SMILES: CCCCCCCCCCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl laurate (4c)

According to General Procedure A, silyl ether 3 (997 mg, 2.50 mmol), lauric acid (601 mg, 3.00 mmol), DCC (619 mg, 3.00 mmol) and DMAP (458 mg, 3.75 mmol) in CH₂Cl₂ (8 mL) provided ester 4c (1.38 g, quantitative yield) as a clear, colourless oil.

R_{f}=0.56 (SiO₂, 95:5 hexanes/EtOAc);

¹H NMR (300 MHZ, CDCl₃): δ 5.56-5.41 (m, 1H), 5.41-5.26 (m, 1H), 4.90 (quint., J=6.2 Hz, 1H), 3.61 (t, J=6.6 Hz, 2H), 2.37-2.21 (m, 4H), 2.11-1.95 (m, 2H), 1.72-1.43 (m, 12H), 1.43-1.13 (m, 38H), 0.91 (br s, 15H), 0.07 (s, 6H).

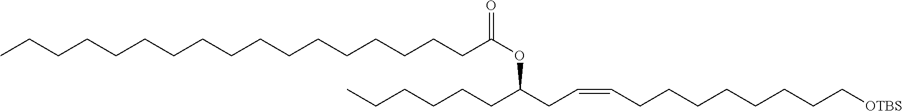 [SMILES: CCCCCCCCCCCCCCCCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl stearate (4d)

According to General Procedure A, silyl ether 3 (997 mg, 2.50 mmol), stearic acid (853 mg, 3.00 mmol), DCC (619 mg, 3.00 mmol) and DMAP (458 mg, 3.75 mmol) in 2:1 THF/CH₂Cl₂ (6 mL) provided ester 4d (1.56 g, 94%) as a clear, colourless oil.

R_{f}=0.48 (SiO₂, 90:10 hexanes/EtOAc);

¹H NMR (300 MHz, CDCl₃): δ 5.57-5.41 (m, 1H), 5.41-5.25 (m, 1H), 4.90 (quint., J=6.3 Hz, 1H), 3.61 (t, J=6.5 Hz, 2H), 2.39-2.20 (m, 4H), 2.11-1.96 (m, 2H), 1.72-1.43 (m, 8H), 1.43-1.13 (m, 44H), 0.91 (br s, 15H), 0.07 (s, 6H).

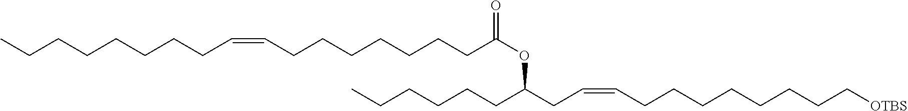 [SMILES: CCCCCCCC/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl oleate (4e)

According to General Procedure A, silyl ether 3 (997 mg, 2.50 mmol), oleic acid (847 mg, 3.00 mmol), DCC (619 mg, 3.00 mmol) and DMAP (458 mg, 3.75 mmol) in CH₂Cl₂ (10 mL) provided ester 4e (1.64 g, quantitative) as a clear, colourless oil.

R_{f}=0.41 (SiO₂, 95:5 hexanes/EtOAc);

¹H NMR (300 MHz, CDCl₃): δ 5.56-5.25 (m, 4H), 4.90 (quint., J=6.2 Hz, 1H), 3.61 (t, J=6.5 Hz, 2H), 2.42-2.19 (m, 8H), 2.11-1.93 (m, 6H), 1.70-1.44 (m, 8H), 1.44-1.17 (m, 40H), 0.91 (br s, 15H), 0.06 (s, 6H).

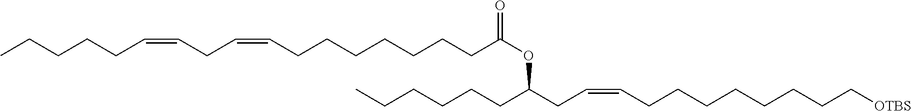 [SMILES: CCCCC/C=C\C/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl linoleate (4f)

According to General Procedure A, silyl ether 3 (847 mg, 2.12 mmol), linoleic acid (715 mg, 2.55 mmol), DCC (526 mg, 2.55 mmol) and DMAP (389 mg, 3.19 mmol) in CH₂Cl₂ (7 mL) provided ester 4f (1.06 g, 76%) as a clear, colourless oil.

R_{f}=0.46 (SiO₂, 95:5 hexanes/EtOAc);

¹H NMR (300 MHz, CDCl₃): δ 5.67-5.24 (m, 6H), 4.90 (quint., J=6.2 Hz, 1H), 3.61 (t, J=6.6 Hz, 2H), 2.79 (t, J=5.9 Hz, 2H), 2.40-2.17 (m, 4H), 2.15-1.94 (m, 4H), 1.71-1.44 (m, 8H), 1.43-1.17 (m, 26H), 0.91 (br s, 15H), 0.07 (s, 6H).

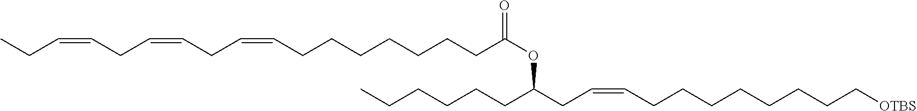 [SMILES: CC/C=C\C/C=C\C/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl linolenate (4g)

According to General Procedure A, silyl ether 3 (997 mg, 2.50 mmol), linolenic acid (835 mg, 3.00 mmol), DCC (619 mg, 3.00 mmol) and DMAP (458 mg, 3.75 mmol) in CH₂Cl₂ (8 mL) provided ester 4g (1.52 g, 92%) as a clear, colourless oil.

R_{f}=0.34 (SiO₂, 95:5 hexanes/EtOAc);

¹H NMR (300 MHz, CDCl₃): δ 5.58-5.26 (m, 8H), 4.90 (quint., J=6.2 Hz, 1H), 3.61 (t, J=6.5 Hz, 2H), 2.83 (t, J=5.8 Hz, 4H), 2.35-2.22 (m, 4H), 2.17-1.97 (m, 6H), 1.69-1.44 (m, 6H), 1.43-1.18 (m, 26H), 1.00 (t, J=7.5 Hz, 3H), 0.91 (br s, 12H), 0.07 (s, 6H).

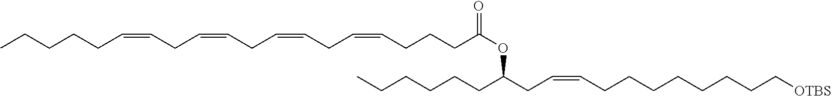 [SMILES: CCCCC/C=C\C/C=C\C/C=C\C/C=C\CCCC(=O)O[C@@H](C/C=C\CCCCCCCCO[Si](C)(C)C(C)(C)C)CCCCCC]

### (R,Z)-18-((tert-Butyldimethylsilyl)oxy) octadec-9-en-7-yl arachidonate (4h)

According to General Procedure A, silyl ether 3 (797 mg, 2.00 mmol), arachidonic acid (670 mg, 2.20 mmol), DCC (227 mg, 2.20 mmol) and DMAP (366 mg, 3.00 mmol) in CH₂Cl₂ (7 mL) provided ester 4h (730 mg, 53%) as a clear, colourless oil after flash column chromatography (99:1→95:5 hexanes/EtOAc).

R_{f}=0.57 (SiO₂, 95:5 hexanes/EtOAc);

¹H NMR (300 MHz, CDCl₃): δ 5.57-5.26 (m, 10H), 4.91 (quint., J=6.3 Hz, 1H), 3.61 (t, J=6.5 Hz, 2H), 2.94-2.84 (m, 6H), 2.38-2.22 (m, 4H), 2.20-1.96 (m, 6H), 1.71 (quint., J=7.4 Hz, 2H), 1.63-1.46 (m, 4H), 1.45-1.16 (m, 26H), 0.91 (br s, 15H), 0.07 (s, 6H).

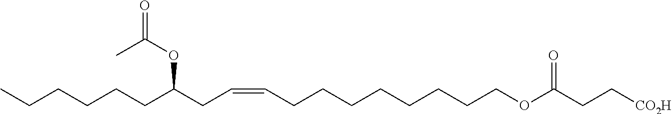 [SMILES: CCCCCC[C@H](C/C=C\CCCCCCCCOC(=O)CCC(=O)O)OC(C)=O]

### (R,Z)-4-((12-Acetoxyoctadec-9-en-1-yl)oxy)-4-oxobutanoic acid (5a)

According to General Procedure B, desilylation of silyl ether 4a (1.79 g, 4.07 mmol) with HF·pyridine solution (1.52 mL, 12.2 mmol), pyridine (0.98 mL, 12.2 mmol) and THF (10 mL) gave the intermediate primary alcohol (1.34 g), which was subjected to acylation with succinic anhydride (814 mg, 8.14 mmol), DMAP (1.24 g, 10.2 mmol) and CH₂Cl₂ (10 mL) to afford carboxylic acid 5a (1.72 g, quantitative yield).

R_{f}=0.23 (SiO₂, 50:50 hexanes/EtOAc);

¹H NMR (300 MHz, CDCl₃): δ 5.57-5.42 (m, 1H), 5.42-5.27 (m, 1H), 4.89 (quin., J=6.2 Hz, 1H), 4.11 (t, J=6.7 Hz, 2H), 2.76-2.57 (m, 4H), 2.11-1.97 (m, 2H), 2.05 (s, 3H), 1.72-1.46 (m, 4H), 1.46-1.16 (m, 18H), 0.90 (m, 3H).

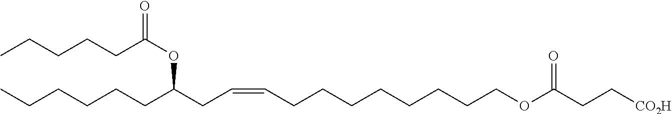 [SMILES: CCCCCC[C@H](C/C=C\CCCCCCCCOC(=O)CCC(=O)O)OC(=O)CCCCC]

### (R,Z)-4-((12-(Hexanoyloxy) octadec-9-en-1-yl)oxy)-4-oxobutanoic acid (5b)

According to General Procedure B, desilylation of silyl ether 4b (2.35 g, 5.00 mmol) with HF·pyridine solution (1.86 mL, 15.0 mmol), pyridine (1.21 mL, 15.0 mmol) and THF (13 mL) gave the intermediate primary alcohol (2.01 g), which was subjected to acylation with succinic anhydride (1.00 g, 10.0 mmol), DMAP (1.53 g, 12.5 mmol) and CH₂Cl₂ (13 mL) to afford carboxylic acid 5b (2.20 g, 92% yield).

R_{f}=0.32 (SiO₂, 50:50 hexanes/EtOAc);

¹H NMR (300 MHz, CDCl₃): δ 5.56-5.42 (m, 1H), 5.41-5.27 (m, 1H), 4.90 (quint., J=6.4 Hz, 1H), 4.11 (t, J=6.5 Hz, 2H), 2.76-2.58 (m, 4H), 2.38-2.22 (m, 4H), 2.11-1.96 (m, 2H), 1.73-1.47 (m, 6H), 1.46-1.15 (m, 22H), 0.97-0.82 (m, 6H).

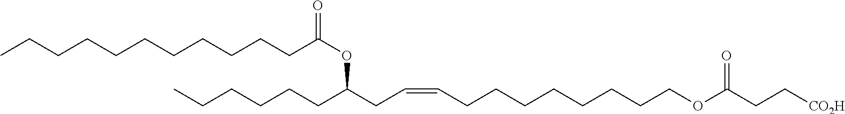 [SMILES: CCCCCCCCCCCC(=O)O[C@@H](C/C=C\CCCCCCCCOC(=O)CCC(=O)O)CCCCCC]

### (R,Z)-4-((12-(Lauroyloxy) octadec-9-en-1-yl)oxy)-4-oxobutanoic acid (5c)

According to General Procedure B, desilylation of silyl ether 4c (1.38 g, 2.50 mmol) with HF·pyridine solution (0.93 mL, 7.50 mmol), pyridine (0.60 mL, 7.50 mmol) and THF (8 mL) gave the intermediate primary alcohol (1.21 g), which was subjected to acylation with succinic anhydride (500 mg, 5.00 mmol), DMAP (764 mg, 6.25 mmol) and CH₂Cl₂ (8 mL) to afford carboxylic acid 5c (1.33 g, 94%).

R_{f}=0.44 (SiO₂, 50:50 hexanes/EtOAc);

¹H NMR (300 MHz, CDCl₃): δ 5.57-5.42 (m, 1H), 5.41-5.26 (m, 1H), 4.90 (quint., J=6.2 Hz, 1H), 4.12 (t, J=6.6 Hz, 2H), 2.78-2.59 (m, 4H), 2.37-2.22 (m, 4H), 2.11-1.96 (m, 2H), 1.73-1.45 (m, 6H), 1.45-1.12 (m, 28H), 0.98-0.80 (m, 6H).

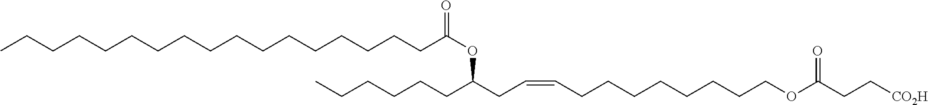 [SMILES: CCCCCCCCCCCCCCCCCC(=O)O[C@@H](C/C=C\CCCCCCCCOC(=O)CCC(=O)O)CCCCCC]

### (R,Z)-4-oxo-4-((12-(Stearoyloxy) octadec-9-en-1-yl)oxy) butanoic acid (5d)

According to General Procedure B, desilylation of silyl ether 4d (1.66 g, 2.50 mmol) with HF·pyridine solution (0.93 mL, 7.50 mmol), pyridine (0.60 mL, 7.50 mmol) and THF (8 mL) gave the intermediate primary alcohol (1.30 g), which was subjected to acylation with succinic anhydride (500 mg, 5.00 mmol), DMAP (764 mg, 6.25 mmol) and CH₂Cl₂ (8 mL) to afford carboxylic acid 5d (1.29 g, 79% yield).

R_{f}=0.35 (SiO₂, 50:50 hexanes/EtOAc);

¹H NMR (300 MHZ, CDCl₃): δ 5.56-5.42 (m, 1H), 5.41-5.27 (m, 1H), 4.90 (quint., J=6.3 Hz, 1H), 4.11 (t, J=6.5 Hz, 2H), 2.77-2.58 (m, 4H), 2.39-2.19 (m, 4H), 2.12-1.95 (m, 2H), 1.73-1.45 (m, 6H), 1.44-1.11 (m, 46H), 0.98-0.80 (m, 6H).

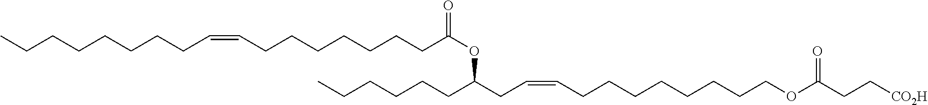 [SMILES: CCCCCCCC/C=C\CCCCCCCC(=O)O[C@@H](C/C=C\CCCCCCCCOC(=O)CCC(=O)O)CCCCCC]

### (R,Z)-4-oxo-4-((12-(Oleoyloxy) octadec-9-en-1-yl)oxy) butanoic acid (5e)

According to General Procedure B, desilylation of silyl ether 4e (663 mg, 1.00 mmol) with HF·pyridine solution (0.37 mL, 3.00 mmol), pyridine (0.24 mL, 3.00 mmol) and THF (5 mL) gave the intermediate primary alcohol (546 mg), which was subjected to acylation with succinic anhydride (200 mg, 2.00 mmol), DMAP (305 mg, 2.50 mmol) and CH₂Cl₂ (5 mL) to afford carboxylic acid 5e (630 mg, 97% yield).

R_{f}=0.42 (SiO₂, 50:50 hexanes/EtOAc);

¹H NMR (300 MHz, CDCl₃): δ 5.57-5.25 (m, 4H), 4.90 (quint., J=6.2 Hz, 1H), 4.11 (t, J=6.5 Hz, 2H), 2.77-2.59 (m, 4H), 2.39-2.20 (m, 4H), 2.13-1.93 (m, 6H), 1.72-1.46 (m, 6H), 1.46-1.02 (m, 34H), 0.97-0.80 (m, 6H).

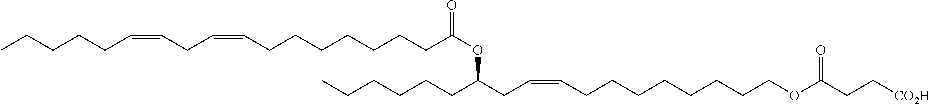 [SMILES: CCCCC/C=C\C/C=C\CCCCCCCC(=O)O[C@@H](C/C=C\CCCCCCCCOC(=O)CCC(=O)O)CCCCCC]

### 4-(((R,Z)-12-(Linoleoyloxy) octadec-9-en-1-yl)oxy)-4-oxobutanoic acid (5f)

According to General Procedure B, desilylation of silyl ether 4f (1.06 g, 1.60 mmol) with HF·pyridine solution (0.60 mL, 4.80 mmol), pyridine (0.39 mL, 4.80 mmol) and THF (8 mL) gave the intermediate primary alcohol (890 mg), which was subjected to acylation with succinic anhydride (320 mg, 3.20 mmol), DMAP (489 mg, 4.00 mmol) and CH₂Cl₂ (8 mL) to afford carboxylic acid 5f (1.04 g, quantitative yield).

R_{f}=0.35 (SiO₂, 50:50 hexanes/EtOAc);

¹H NMR (300 MHz, CDCl₃): δ 5.57-5.26 (m, 6H), 4.90 (quint., J=6.3 Hz, 1H), 4.11 (t, J=6.7 Hz, 2H), 2.79 (t, J=6.0 Hz, 2H), 2.75-2.58 (m, 6H), 2.38-2.20 (m, 4H), 2.14-1.94 (m, 6H), 1.72-1.46 (m, 8H), 1.46-1.14 (m, 30H), 0.98-0.81 (m, 6H).

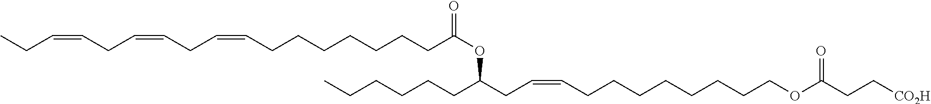 [SMILES: CC/C=C\C/C=C\C/C=C\CCCCCCCC(=O)O[C@@H](C/C=C\CCCCCCCCOC(=O)CCC(=O)O)CCCCCC]

### 4-(((R,Z)-12-(Linolenoyloxy) octadec-9-en-1-yl)oxy)-4-oxobutanoic acid (5g)

According to General Procedure B, desilylation of silyl ether 4g (1.54 g, 2.34 mmol) with HF·pyridine solution (0.87 mL, 7.01 mmol), pyridine (0.57 mL, 7.01 mmol) and THF (6 mL) gave the intermediate primary alcohol (1.31 g), which was subjected to acylation with succinic anhydride (468 mg, 4.68 mmol), DMAP (714 mg, 5.84 mmol) and CH₂Cl₂ (6 mL) to afford carboxylic acid 5g (1.47 g, quantitative yield).

R_{f}=0.35 (SiO₂, 50:50 hexanes/EtOAc);

¹H NMR (300 MHz, CDCl₃): δ 5.56-5.25 (m, 8H), 4.90 (quint., J=6.2 Hz, 1H), 4.11 (t, J=6.5 Hz, 2H), 2.82 (t, J=5.7 Hz, 4H), 2.37-2.22 (m, 4H), 2.16-1.95 (m, 6H), 1.74-1.46 (m, 6H), 1.46-1.15 (m, 30H), 0.99 (t, J=7.6 Hz, 3H), 0.94-0.83 (m, 6H).

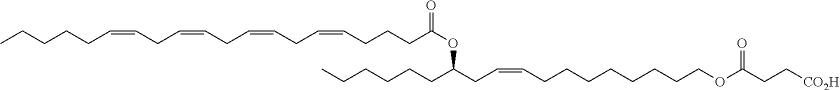 [SMILES: CCCCC/C=C\C/C=C\C/C=C\C/C=C\CCCC(=O)O[C@@H](C/C=C\CCCCCCCCOC(=O)CCC(=O)O)CCCCCC]

### 4-(((R,Z)-12-(Arachidonoyloxy) octadec-9-en-1-yl)oxy)-4-oxobutanoic acid (5h)

According to General Procedure B, desilylation of silyl ether 4h (711 mg, 1.04 mmol) with HF·pyridine solution (0.39 mL, 3.11 mmol), pyridine (0.25 mL, 3.11 mmol) and THF (5 mL) gave the intermediate primary alcohol (593 mg), which was subjected to acylation with succinic anhydride (201 mg, 2.01 mmol), DMAP (306 mg, 2.51 mmol) and CH₂Cl₂ (5 mL) to afford carboxylic acid 5h (582 mg, 87% yield).

R_{f}=0.31 (SiO₂, 50:50 hexanes/EtOAc);

¹H NMR (300 MHZ, CDCl₃): δ 5.58-5.24 (m, 10H), 4.90 (quint., J=6.2 Hz, 1H), 4.11 (t, J=6.7 Hz, 2H), 2.93-2.75 (m, 6H), 2.76-2.58 (m, 4H), 2.39-2.22 (m, 4H), 2.20-1.96 (m, 6H), 1.71 (quint., J=7.4 Hz, 2H), 1.69-1.47 (m, 4H), 1.46-1.13 (m, 26H), 0.99-0.80 (m, 6H).

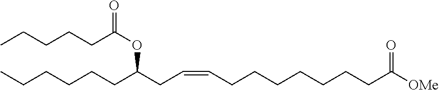 [SMILES: CCCCCC[C@H](C/C=C\CCCCCCCC(=O)OC)OC(=O)CCCCC]

### Methyl (12R)-hexanoyloxyoleate (6a)

According to General Procedure C, methyl ricinoleate (2.00 g, 6.40 mmol), hexanoic acid (898 mg, 7.68 mmol), DCC (1.58 g, 7.68 mmol) and DMAP (1.17 g, 9.60 mmol) in CH₂Cl₂ (10 mL) provided, after filtration through silica gel (95:5 hexanes/EtOAc), ricinoleate 6a (2.52 g, 96% yield) as a clear, colourless oil.

R_{f}: 0.62 (SiO₂, 70:30 hexanes:EtOAc);

¹H (300 MHz, CDCl₃): δ 5.54-5.42 (m, 1H), 5.40-5.28 (m, 1H), 4.90 (quint., J=6.2 Hz, 1H), 3.69 (s, 3H), 2.37-2.23 (m, 6H), 2.11-1.97 (m, 2H), 1.72-1.48 (m, 6), 1.43-1.20 (m, 20), 0.96-0.84 (m, 6H).

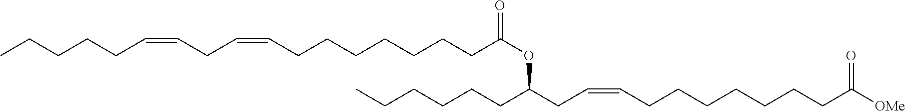 [SMILES: CCCCC/C=C\C/C=C\CCCCCCCC(=O)O[C@@H](C/C=C\CCCCCCCC(=O)OC)CCCCCC]

### Methyl (12R)-linoleoyloxyoleate (6b)

According to General Procedure C, methyl ricinoleate (500 mg, 1.60 mmol), linoleic acid (538 mg, 1.92 mmol), DCC (396 mg, 1.92 mmol) and DMAP (293 mg, 2.40 mmol) in CH₂Cl₂ (5 mL) provided, after filtration through silica gel (95:5 hexanes/EtOAc), ricinoleate 6c (875 g, 93% yield) as a light yellow oil.

R_{f}: 0.67 (SiO₂, 80:20 hexanes:EtOAc);

1H (300 MHz, CDCl₃): δ 5.54-5.42 (m, 1H), 5.40-5.28 (m, 1H), 4.90 (quint., J=6.2 Hz, 1H), 3.69 (s, 3H), 2.37-2.23 (m, 6H), 2.11-1.97 (m, 2H), 1.72-1.48 (m, 6), 1.43-1.20 (m, 20), 0.96-0.84 (m, 6H).

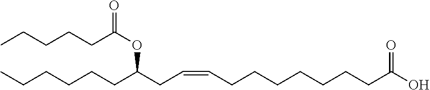 [SMILES: CCCCCC[C@H](C/C=C\CCCCCCCC(=O)O)OC(=O)CCCCC]

### (12R)-Hexanoyloxyoleic acid (7a)

An argon-flushed round bottom flask was charged with methyl ester 6a (1.97 g, 4.79 mmol, 1.00 equiv.) and t-BuOH (12 mL), then aqueous 2.0 M NaOH (1.80 mL, 3.60 mmol, 0.75 equiv.). After 17 h, the pH of the reaction solution was adjusted to 2 using aqueous 1 M HCl and extracted with Et₂O (3×30 mL). The combined organics were washed with water (1×30 mL), brine (1×30 mL), dried over Na₂SO₄ and concentrated on a rotary evaporator under reduced pressure. The residue was filtered through a plug of silica (98:2:0→50:45:5 hexanes:EtOAc:MeOH) to afford carboxylic acid 7a (1.30 g, 92% yield) as a pale yellow oil.

R_{f}=0.24 (SiO₂, 75:20:5 hexanes/EtOAc/MeOH);

¹H NMR (300 MHz, CDCl₃): δ 5.55-5.28 (m, 6H), 4.90 (quint., J=6.2 Hz, 1H), 3.69 (s, 3H), 2.79 (t, J=5.8 Hz, 2H), 2.40-2.21 (m, 6H), 2.16-1.93 (m, 6H), 1.72-1.46 (m, 8H), 1.46-1.18 (m, 32H), 1.00-0.80 (m, 6H).

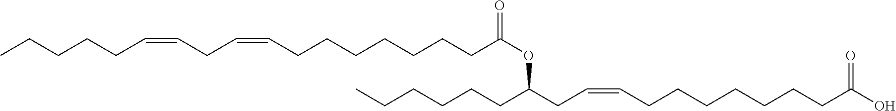 [SMILES: CCCCC/C=C\C/C=C\CCCCCCCC(=O)O[C@@H](C/C=C\CCCCCCCC(=O)O)CCCCCC]

### (12R)-Linoleoyloxyoleic acid (7b)

An argon-flushed round bottom flask was charged with methyl ester 6b (5.97 g, 10.4 mmol, 1.00 equiv.) and t-BuOH (26 mL), then aqueous 2.0 M NaOH (4.70 mL, 9.30 mmol, 0.90 equiv.). After 17 h, the pH of the reaction solution was adjusted to 2 using aqueous 1 M HCl and extracted with Et₂O (3×30 mL). The combined organics were washed with water (1×30 mL), brine (1×30 mL), dried over Na₂SO₄ and concentrated on a rotary evaporator under reduced pressure. The residue was purified by flash column chromatography (SiO₂, 95:5:0→80:15:5 hexanes:EtOAc:MeOH) to afford carboxylic acid 7b (4.48 g, 85% yield) as a pale yellow oil.

R_{f}: 0.35 (SiO₂, 75:20:5 hexanes/EtOAc/MeOH);

¹H (CDCl₃, 300 MHz): δ 5.55-5.28 (m, 6H), 4.90 (quint., J=6.2 Hz, 1H), 2.79 (t, J=6.0 Hz, 2H), 2.43-2.21 (m, 6H), 2.14-1.96 (m, 6H), 1.73-1.47 (m, 6H), 1.46-1.18 (m, 30H), 0.99-0.81 (m, 6H).

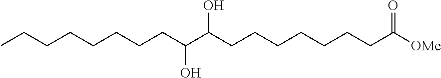 [SMILES: CCCCCCCCC(O)C(O)CCCCCCCC(=O)OC]

### Methyl 9,10-dihydroxystearate (8)

KOH (7.01 g, 125 mmol, 5.00 equiv.) was added to a rapidly stirred room temperature mixture of oleic acid (7.06 g, 25.0 mmol) and water (175 mL) in a 500 mL Erlenmeyer flask, then cooled to ˜10° C. A solution of KMnO₄ (7.11 g, 45.0 mmol, 1.80 equiv.) in water (75 mL) was added dropwise over 10 min. After stirring an additional 10-15 min, the reaction was quenched by addition of saturated aqueous NaHSO₃, then adjusted to pH≤2 by addition of concentrated HCl with the aid of a cooling bath. The white, flocculent mixture was stirred for 1 h at room temperature, then the solids collected by suction filtration and dried in air overnight. The resulting white solids were hot gravity filtered and recrystallized from EtOH to afford the (±)-syn-9,10-dihydroxystearic acid as white crystals (5.86 g, 74% yield).

Concentrated H₂SO₄ (0.06 mL, 1.00 mmol, 0.05 equiv.) was added to a MeOH (50 mL) suspension of the above dihydroxy acid (6.33 g, 20.0 mmol) and the resulting mixture was heated at reflux. After 14 h, the mixture was cooled to room temperature and concentrated on a rotary evaporator under reduced pressure and the resulting residue was partitioned between EtOAc and saturated aqueous NaHCO₃. The organic layer was washed with water (1×75 mL), brine, dried over Na₂SO₄ and concentrated on a rotary evaporator under reduced pressure to afford methyl ester 8 (6.44 g, 97% yield) as a white solid.

R_{f}=0.45 (SiO₂, 50:50 hexanes/EtOAc);

¹H NMR (300 MHz, CDCl₃): δ 3.68 (s, 3H), 3.61 (app br s, 2H), 2.32 (t, J=7.4 Hz, 2H), 2.06-1.85 (app br s, 2H), 1.73-1.16 (m, 26H), 0.96-0.81 (m, 3H).

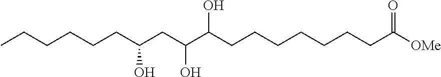 [SMILES: CCCCCC[C@@H](O)CC(O)C(O)CCCCCCCC(=O)OC]

### Methyl 9,10,12R-trihydroxystearate (9)

KOH (5.61 g, 100 mmol, 2.00 equiv.) was added to a rapidly stirred room temperature mixture of ricinoleic acid (14.9 g, 50.0 mmol) and water (500 mL) in a 1 L Erlenmeyer flask, then cooled to ˜10° C. A solution of KMnO₄ (13.4 g, 85.0 mmol, 1.70 equiv.) in water (250 mL) was added dropwise over 15 min. After stirring an additional 10-15 min, the reaction was quenched by addition of saturated aqueous Na₂SO₃, then adjusted to pH≤2 by addition of concentrated HCl with the aid of a cooling bath. The white, flocculent mixture was stirred for 4 h at room temperature, then the solids collected by suction filtration and dried in air overnight. The resulting white solids were hot gravity filtered with EtOH to afford the crude 9,10,12-trihydroxystearic acid, which was used without further purification.

Concentrated H₂SO₄ (0.13 mL, 2.50 mmol, 0.05 equiv.) was added to a MeOH (120 mL) suspension of the above dihydroxy acid (6.33 g, 20.0 mmol) and the resulting mixture was heated at reflux. After 14 h, the mixture was cooled to room temperature and concentrated on a rotary evaporator under reduced pressure and the resulting residue was partitioned between warm EtOAc and saturated aqueous NaHCO₃. The organic layer was washed with water (1×75 mL), brine, dried over Na₂SO₄ and concentrated on a rotary evaporator under reduced pressure. The resulting pale yellow solid was triturated four times with warm Et₂O to afford methyl ester 9 (9.52 g, 55% yield) as a white solid.

R_{f}=0.33 (SiO₂, 50:50 hexanes/EtOAc);

¹H NMR (300 MHz, CDCl₃): δ 4.07-3.58 (m, 3H), 3.68 (s, 3H), 2.31 (t, J=7.5 Hz, 2H), 1.86-1.14 (m, 24H), 0.90 (br t, 3H).

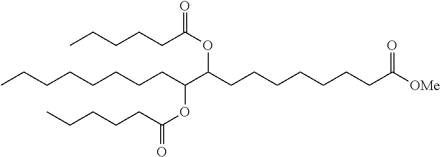 [SMILES: CCCCCCCCC(OC(=O)CCCCC)C(CCCCCCCC(=O)OC)OC(=O)CCCCC]

### Methyl 9,10-dihexanoyloxystearate (10a)

DCC (2.27 g, 11.0 mmol, 2.20 equiv.) was added to a stirring, ice-cold CH₂Cl₂ (13 mL) solution hexanoic acid (1.28 g, 11.0 mmol, 2.20 equiv.) in a round bottom flask under argon, then the ice bath was removed and the resultant stirred for 15 min. The reaction mixture was cooled again in an ice bath, diol 8 (1.65 g, 5.00 mmol) was added, followed by DMAP (1.53 g, 12.5 mmol, 2.50 equiv.), and the reaction mixture was allowed to warm to room temperature over 14 h. The reaction mixture was diluted with Et₂O, stirred for 10 min, then filtered through Celite®. The filtrate was washed with aqueous 1 M HCl (2×30 mL), aqueous 1 M NaOH (2×30 mL), H₂O (1×30 mL), brine, dried over Na₂SO₄ and concentrated on a rotary evaporator under reduced pressure to afford triester 10a (2.61 g, quantitative yield) as a clear, colourless oil.

R_{f}=0.66 (SiO₂, 70:30 hexanes/EtOAc);

¹H NMR (300 MHz, CDCl₃): δ 5.08-4.92 (m, 2H), 3.68 (s, 3H), 2.40-2.20 (m, 6H), 1.74-1.44 (m, 12H), 1.44-1.13 (m, 28H), 1.01-0.80 (m, 9H).

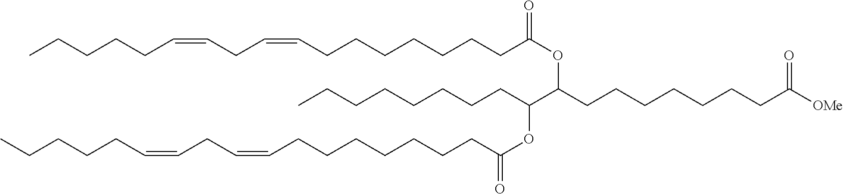 [SMILES: CCCCC/C=C\C/C=C\CCCCCCCC(=O)OC(CCCCCCCC)C(CCCCCCCC(=O)OC)OC(=O)CCCCCCC/C=C\C/C=C\CCCCC]

### Methyl 9,10-dilinoleoyloxystearate (10b)

DCC (4.33 g, 21.0 mmol, 2.10 equiv.) was added to a stirring, ice-cold CH₂Cl₂ (25 mL) solution linoleic acid (5.89 g, 21.0 mmol, 2.20 equiv.) in a round bottom flask under argon, then the ice bath was removed and the resultant stirred for 15 min. The reaction mixture was cooled again in an ice bath, diol 8 (3.30 g, 10.0 mmol) was added, followed by DMAP (3.05 g, 25.0 mmol, 2.50 equiv.), and the reaction mixture was allowed to warm to room temperature over 14 h. The reaction mixture was diluted with hexanes, stirred for 10 min, then filtered through Celite®. The filtrate was concentrated on a rotary evaporator to yield the crude as a white semi-solid, which was purified by filtration through a plug of silica gel (95:5 hexanes/EtOAc) to afford the triester 10b (7.24 g, 85% yield) as a clear colourless oil.

R_{f}=0.57 (SiO₂, 70:30 hexanes/EtOAc);

¹H NMR (300 MHz, CDCl₃): δ 5.49-5.27 (m, 8H), 5.05-4.94 (m, 2H), 3.68 (s, 3H), 2.79 (t, J=5.9 Hz, 4H), 2.39-2.23 (m, 6H), 2.15-1.97 (m, 8H), 1.72-1.45 (m, 10H), 1.45-1.15 (m, 50H), 0.98-0.82 (m, 9H).

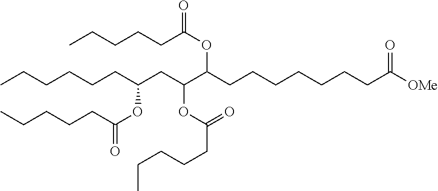 [SMILES: CCCCCC[C@H](CC(OC(=O)CCCCC)C(CCCCCCCC(=O)OC)OC(=O)CCCCC)OC(=O)CCCCC]

### Methyl 9,10,12R-trihexanoyloxystearate (11)

DCC (2.64 g, 12.8 mmol, 3.20 equiv.) was added to a stirring, ice-cold CH₂Cl₂ (13 mL) solution hexanoic acid (1.49 g, 12.8 mmol, 3.20 equiv.) in a round bottom flask under argon, then the ice bath was removed and the resultant stirred for 15 min. The reaction mixture was cooled again in an ice bath, triol 9 (1.39 g, 4.00 mmol) was added, followed by DMAP (1.71 g, 14.0 mmol, 3.50 equiv.), and the reaction mixture was allowed to warm to room temperature over 14 h. The reaction mixture was diluted with hexanes, stirred for 10 min, then filtered through Celite®. The filtrate was washed with aqueous 1 M HCl (2×30 mL), aqueous 1 M NaOH (2×30 mL), H₂O (1×30 mL), brine, dried over Na₂SO₄ and concentrated on a rotary evaporator under reduced pressure to afford triester 11 (1.99 g, 78% yield) as a clear, colourless oil.

R_{f}=0.77 (SiO₂, 70:30 hexanes/EtOAc);

¹H NMR (300 MHz, CDCl₃): δ 5.13-4.84 (m, 3H), 3.68 (s, 3H), 2.38-2.19 (m, 8H), 1.92-1.69 (m, 2H), 1.69-1.42 (m, 12H), 1.42-1.16 (m, 28H), 1.00-0.82 (m, 12H).

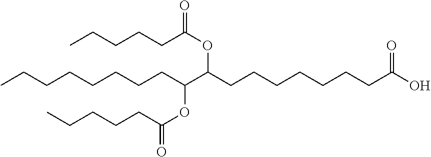 [SMILES: CCCCCCCCC(OC(=O)CCCCC)C(CCCCCCCC(=O)O)OC(=O)CCCCC]

### 9,10-Dihexanoyloxystearic acid (12a)

Aqueous 2.0 M KOH (0.91 mL, 1.82 mmol, 1.00 equiv.) was added to a room temperature t-BuOH (7 mL) solution of triester 10a (1.05 g, 2.00 mmol, 1.10 equiv.) in a round bottom flask under argon. After stirring for 20 h, the reaction mixture was acidified to pH≤2 by addition of aqueous 3 M HCl and extracted with Et₂O (3×20 mL). The combined organic layers were washed with brine, dried over Na₂SO₄ and concentrated on a rotary evaporator under reduced pressure. The crude residue was purified by flash column chromatography (90:5:5→85:10:5 hexanes/EtOAc/MeOH) to afford carboxylic acid 12a (802 mg, 86% yield) as a clear, colourless oil.

R_{f}=0.22 (SiO₂, 85:10:5 hexanes/EtOAc/MeOH);

¹H NMR (300 MHz, CDCl₃): δ 5.08-4.93 (m, 2H), 2.36 (t, J=7.8 Hz, 2H), 2.30 (t, J=7.6 Hz, 4H), 1.72-1.44 (m, 10H), 1.44-1.16 (m, 30H), 0.97-0.83 (m, 9H).

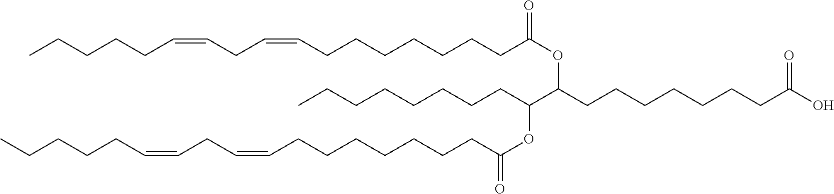 [SMILES: CCCCC/C=C\C/C=C\CCCCCCCC(=O)OC(CCCCCCCC)C(CCCCCCCC(=O)O)OC(=O)CCCCCCC/C=C\C/C=C\CCCCC]

### 9,10-Dilinoleoyloxystearic acid (12b)

Aqueous 2.0 M KOH (3.00 mL, 6.00 mmol, 1.00 equiv.) was added to a room temperature t-BuOH (7 mL) solution of triester 10b (5.64 g, 6.60 mmol, 1.10 equiv.) in a round bottom flask under argon. After stirring for 20 h, the reaction mixture was acidified to pH≤2 by addition of aqueous 3 M HCl and extracted with hexanes (3×75 mL). The combined organic layers were washed with brine, dried over Na₂SO₄ and concentrated on a rotary evaporator under reduced pressure. The crude residue was purified by flash column chromatography (90:10:0→85:10:5 hexanes/EtOAc/MeOH) to afford carboxylic acid 12b (2.39 g, 68% yield) as a clear, colourless oil.

R_{f}=0.33 (SiO₂, 85:10:5 hexanes/EtOAc/MeOH);

¹H NMR (300 MHZ, CDCl₃): δ 5.49-5.25 (m, 8H), 5.07-4.93 (m, 2H), 2.79 (t, J=5.9 Hz, 4H), 2.36 (t, J=7.7 Hz, 2H), 2.30 (t, J=7.5 Hz, 4H), 2.13-2.00 (m, 8H), 1.72-1.45 (m, 10H), 1.45-1.15 (m, 50H), 0.98-0.81 (m, 9H).

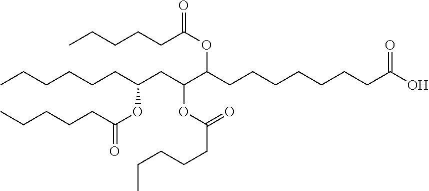 [SMILES: CCCCCC[C@H](CC(OC(=O)CCCCC)C(CCCCCCCC(=O)O)OC(=O)CCCCC)OC(=O)CCCCC]

### 9,10,12R-Trihexanoyloxystearic acid (13)

Aqueous 2.0 M KOH (1.47 mL, 2.94 mmol, 1.00 equiv.) was added to a room temperature t-BuOH (10 mL) solution of tetraester 11 (1.98 g, 3.10 mmol, 1.10 equiv.) in a round bottom flask under argon. After stirring for 20 h, the reaction mixture was acidified to pH≤2 by addition of aqueous 3 M HCl and extracted with hexanes (3×30 mL). The combined organic layers were washed with brine, dried over Na₂SO₄ and concentrated on a rotary evaporator under reduced pressure. The crude residue was purified by flash column chromatography (90:10:0→85:10:5→75:20:5 hexanes/EtOAc/MeOH) to afford carboxylic acid 13 (1.40 g, 78% yield) as a clear, colourless oil.

R_{f}=0.32 (SiO₂, 80:15:5 hexanes/EtOAc/MeOH);

¹H NMR (300 MHZ, CDCl₃): δ 5.13-4.82 (m, 3H), 2.42-2.18 (m, 8H), 1.92-1.69 (m, 2H), 1.69-1.43 (m, 12H), 1.43-1.14 (m, 28H), 0.99-0.81 (m, 12H).

### Example E: Synthesis of INT-D045

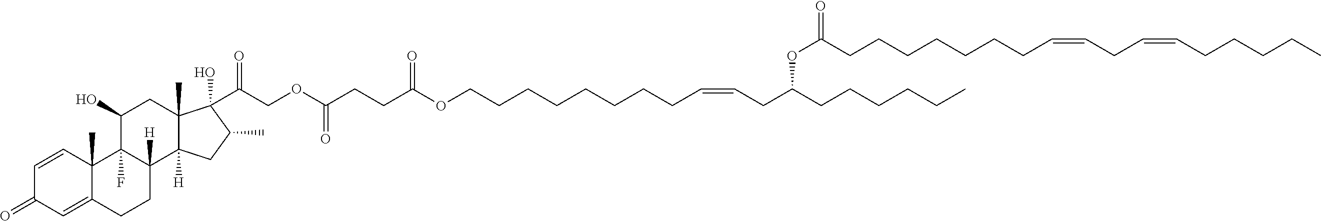

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(linoleoyloxy) octadec-9-en-1-yl) succinate (INT-D045)

According to General Procedure D, dexamethasone (157 mg, 0.40 mmol), hemisuccinate 5f (310 mg, 0.48 mmol), DCC (99 mg, 0.48 mmol), DMAP (73 mg, 0.60 mmol) and CH₂Cl₂ (2 mL) afforded, after flash column chromatography (SiO₂, 80:20→50:50 hexanes/EtOAc), INT-D045 (278 mg, 68% yield) as a clear, colourless oil.

R_{f}=0.50 (SiO₂, 50:50 hexanes/EtOAc);

¹H NMR (300 MHz, CDCl₃): δ 7.22 (d, J=10.1 Hz), 6.36 (dd, J=10.2, 1.8 Hz), 6.13 (s, 1H), 5.56-5.25 (m, 6H), 4.93 (s, 2H), 4.89 (quint., J=6.3 Hz), 4.46-4.31 (m, 1H), 4.10 (t, J=6.8 Hz, 2H), 3.20-3.04 (m, 1H), 2.88-2.54 (m, 7H), 2.53-1.91 (m, 15H), 1.90-1.46 (m, 14H), 1.47-1.12 (m, 34H), 1.06 (s, 3H), 0.99-0.81 (m, 9H).

### Example S: Synthesis of INT-D053

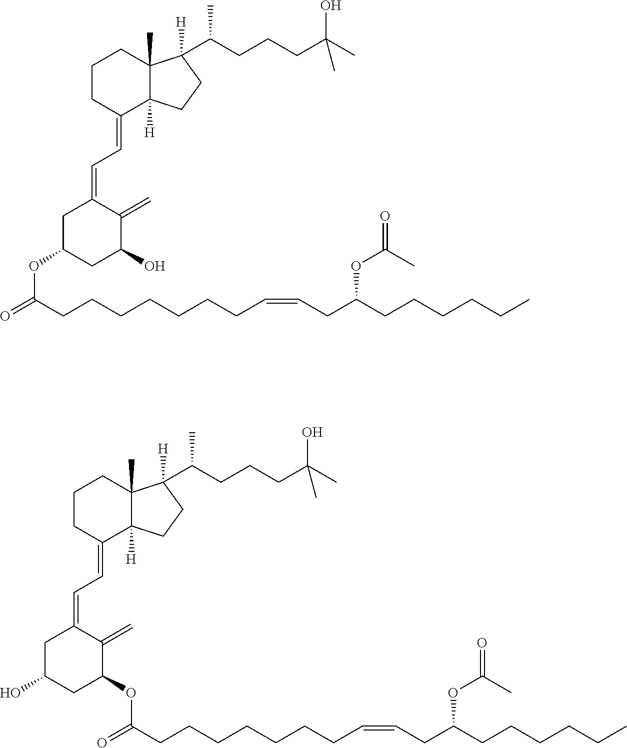 [SMILES: C=C1/C(=C\C=C2/CCC[C@]3(C)[C@@H]([C@H](C)CCCC(C)(C)O)CC[C@@H]23)C[C@@H](OC(=O)CCCCCCC/C=C\C[C@@H](CCCCCC)OC(C)=O)C[C@@H]1O ; C=C1/C(=C\C=C2/CCC[C@]3(C)[C@@H]([C@H](C)CCCC(C)(C)O)CC[C@@H]23)C[C@@H](O)C[C@@H]1OC(=O)CCCCCCC/C=C\C[C@@H](CCCCCC)OC(C)=O]

### (1R,3S,Z)-3-hydroxy-5-(2-((1R,3aS,7aR,E)-1-((R)-6-hydroxy-6-methylheptan-2-yl)-7a-methyloctahydro-4H-inden-4-ylidene)ethylidene)-4-methylenecyclohexyl (R,Z)-12-acetoxyoctadec-9-enoate and (1S,5R,Z)-5-hydroxy-3-(2-((1R,3aS,7aR,E)-1-((R)-6-hydroxy-6-methylheptan-2-yl)-7a-methyloctahydro-4H-inden-4-ylidene)ethylidene)-2-methylenecyclohexyl (R,Z)-12-acetoxyoctadec-9-enoate (INT-D053): JZ-25-057, 029

DCC (50 mg, 0.24 mmol, 1.20 equiv.) was added to a stirring, ice-cold 1:1 CH₂Cl₂/THF (4 mL) solution of (12R)-acetoxyoleic acid (82 mg, 0.24 mmol, 1.20 equiv.) in a round bottom flask under argon, then the ice bath was removed and the resultant stirred for 15 min. The reaction mixture was cooled again in an ice bath and solid calcitriol (83 mg, 0.20 mmol) and DMAP (29 mg, 0.24 mmol, 1.20 equiv.) were added. The reaction mixture was allowed to warm up over 14 h, diluted with EtOAc, stirred for 10 min, then filtered through Celite®. The filtrate was concentrated to afford the crude as a pale yellow oil and subsequently purified by flash column chromatography (SiO₂, 80:20→65:35 hexanes/EtOAc) to afford an ˜1:1 mixture of the 1- and 3-acylated conjugates (61 mg, 41% yield) as a clear, colourless oil.

R_{f}=0.33 (SiO₂, 60:40 hexanes/EtOAc);

¹H NMR (300 MHz, CDCl₃): δ 6.44-6.25 (m, 2H), 6.02 (d, J=11.2 Hz, 1H), 5.92 (d, J=11.2 Hz, 1H), 5.56-5.40 (m, 3H), 5.40-5.27 (m, 4H), 5.26-5.16 (m, 1H), 5.07-4.97 (m, 2H), 4.87 (quint., J=6.2 Hz, 2H), 4.45-4.34 (m, 1H), 4.23-4.10 (m, 1H), 2.89-2.74 (m, 2H), 2.68-2.51 (m, 2H), 2.48-2.18 (m, 11H), 2.17-1.77 (m, 25H), 1.76-1.13 (m, 90H), 1.12-0.99 (m, 2H), 0.99-0.80 (m, 13H), 0.55 (s, 3H), 0.52 (s, 3H).

### Example T: Synthesis of INT-D068

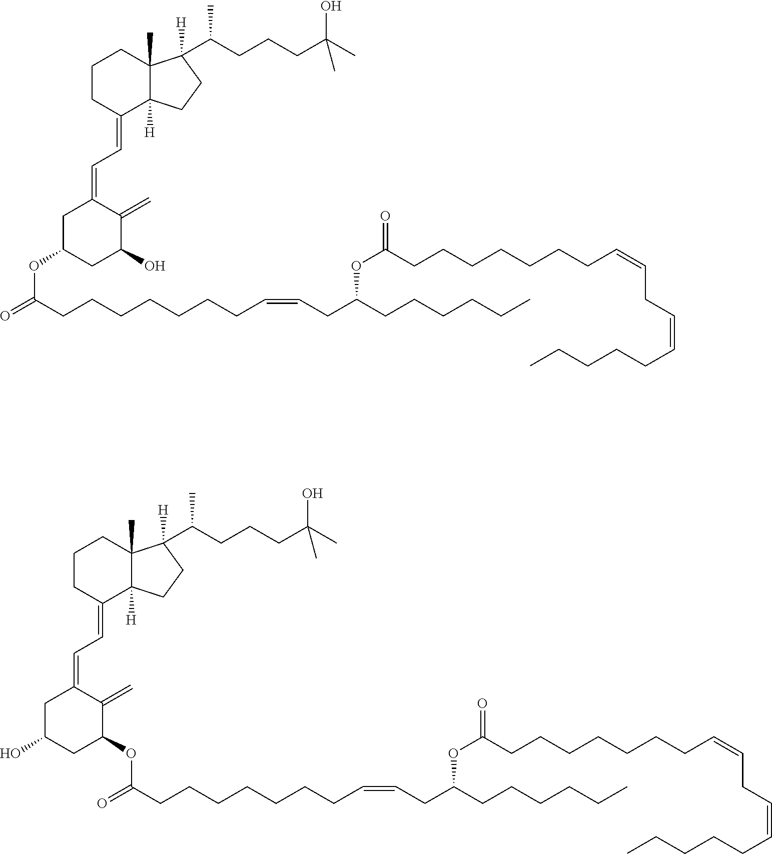 [SMILES: C=C1/C(=C\C=C2/CCC[C@]3(C)[C@@H]([C@H](C)CCCC(C)(C)O)CC[C@@H]23)C[C@@H](OC(=O)CCCCCCC/C=C\C[C@@H](CCCCCC)OC(=O)CCCCCCC/C=C\C/C=C\CCCCC)C[C@@H]1O ; C=C1/C(=C\C=C2/CCC[C@]3(C)[C@@H]([C@H](C)CCCC(C)(C)O)CC[C@@H]23)C[C@@H](O)C[C@@H]1OC(=O)CCCCCCC/C=C\C[C@@H](CCCCCC)OC(=O)CCCCCCC/C=C\C/C=C\CCCCC]

### (R,Z)-18-(((1R,3S,Z)-3-Hydroxy-5-(2-((1R,3aS,7aR,E)-1-((R)-6-hydroxy-6-methylheptan-2-yl)-7a-methyloctahydro-4H-inden-4-ylidene)ethylidene)-4-methylenecyclohexyl)oxy)-18-oxooctadec-9-en-7-yl linoleate and (R,Z)-18-(((1S,5R,Z)-5-hydroxy-3-(2-((1R,3aS,7aR,E)-1-((R)-6-hydroxy-6-methylheptan-2-yl)-7a-methyloctahydro-4H-inden-4-ylidene)ethylidene)-2-methylenecyclohexyl)oxy)-18-oxooctadec-9-en-7-yl linoleate (INT-D068)

DCC (50 mg, 0.24 mmol, 1.20 equiv.) was added to a stirring, ice-cold 1:1 CH₂Cl₂/THF (4 mL) solution of (12R)-linoleoyloxyoleic acid (135 mg, 0.24 mmol, 1.20 equiv.) in a round bottom flask under argon, then the ice bath was removed and the resultant stirred for 15 min. The reaction mixture was cooled again in an ice bath and solid calcitriol (83 mg, 0.20 mmol) and DMAP (29 mg, 0.24 mmol, 1.20 equiv.) were added. The reaction mixture was allowed to warm up over 14 h, diluted with EtOAc, stirred for 10 min, then filtered through Celite®. The filtrate was concentrated to afford the crude as a pale yellow oil and subsequently purified by flash column chromatography (SiO₂, 95:5→90:10→70:30 hexanes/EtOAc) to afford an ˜1:1 mixture of the 1- and 3-acylated conjugates (75 mg, 39% yield) as a clear, colourless oil.

R_{f}=0.26 (SiO₂, 70:30 hexanes/EtOAc);

¹H NMR (300 MHz, CDCl₃): δ 6.43-6.26 (m, 2H), 6.02 (d, J=11.2 Hz, 1H), 5.92 (d, J=11.2 Hz, 1H), 5.57-5.26 (m, 15H), 5.26-5.16 (m, 1H), 5.07-4.97 (m, 2H), 4.88 (quint., J=6.2 Hz, 2H), 4.47-4.34 (m, 1H), 4.23-4.10 (m, 1H), 2.89-2.70 (m, 6H), 2.68-2.52 (m, 2H), 2.47-2.20 (m, 15H), 2.16-1.77 (m, 25H), 1.77-1.12 (m, 118H), 1.12-1.01 (m, 2H), 1.00-0.79 (m, 19H), 0.55 (s, 3H), 0.52 (s, 3H).

### Example W: Synthesis of Disubstituted Calcitriol, INT-D087

An example of a synthesis scheme for preparing a calcitriol lipid conjugate disubstituted with two lipid moieties is provided below:

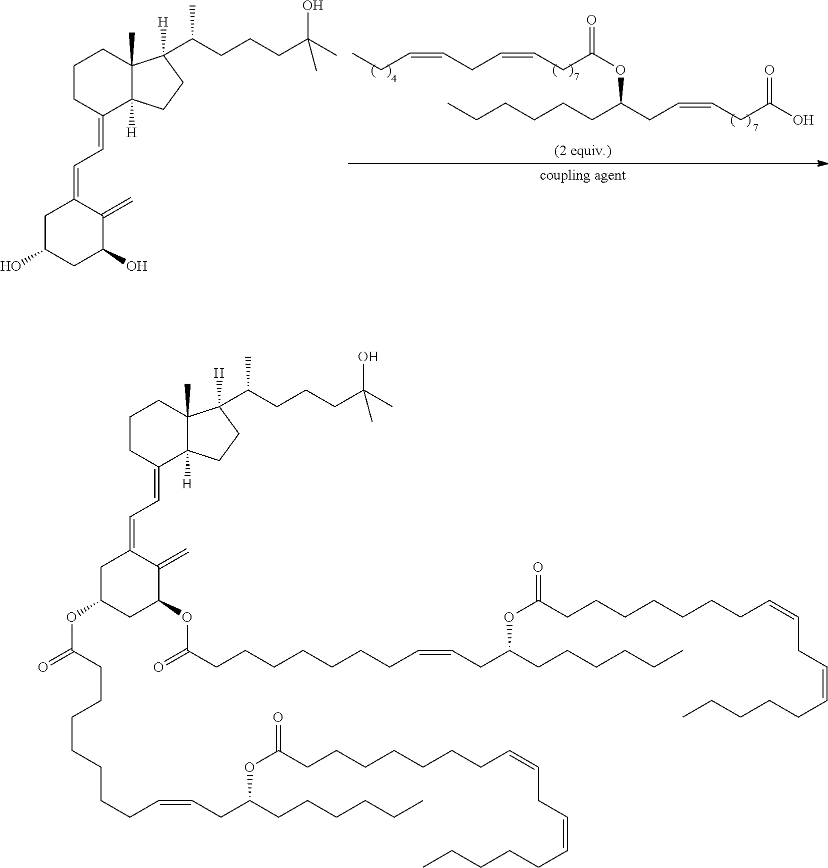

### INT-D087

### Lipid Nanoparticle (LNP) Preparation

The lipids 1,2-distearoyl-sn-glycero-3-phosphocholine (DSPC) or 1,2-dimyristoyl-sn-glycero-3-phosphocholine (DMPC), cholesterol, 1,2-distearoyl-sn-glycero-3-phosphoethanolamine-poly(ethylene glycol) (PEG-DSPE) or 1,2-dimyristoyl-rac-glycero-3-methoxypolyethylene glycol-2000 (PEG-DMG) were dissolved in ethanol. DSPC, DMPC, PEG-DSPE and PEG-DMG were purchased from Avanti Polar Lipids (Alabaster, AL), and cholesterol was obtained from Sigma (St Louis, MO).

Lipid conjugates containing immunomodulatory agents (referred to as “prodrugs” in the following examples) (see FIGS. 10A-10M) were synthesized as provided above and as previously described (e.g. see WO/2020/191477, which is incorporated by reference herein in its entirety).

Lipid conjugates (prodrugs) were dissolved in ethanol, isopropanol, DMSO or THF. LNP were prepared by rapidly mixing DSPC or DMPC, cholesterol, prodrugs, and PEG-DSPE (in a molar ratio of 49/40/10/1) with phosphate-buffered saline (PBS) using a cross-junction mixer. Formulations were dialyzed against PBS to remove residual ethanol. In cases where a peptide antigen, such as ovalbumin peptide 323-329 (OVA), was co-formulated within the LNP, the peptide is dissolved in PBS and rapidly mixed with the lipid phase. Dialysis or tangential flow filtration was used to remove all unentrapped peptide.

To prepare LNPs that contain mRNA that codes for the antigen, ionizable lipid such as INT-A002 (see page 32 of co-owned WO 2021/026647; Application No. PCT/CA2020/051098, which is incorporated herein by reference), DSPC, cholesterol and PEG-DMG were dissolved in ethanol. Prodrugs were dissolved in ethanol, isopropanol, DMSO or THF. The mRNA was dissolved in 10 mM citrate or 25 mM acetate buffer at pH 4.0. LNP were prepared by rapidly mixing the lipid components in ethanol (in molar ratio of 45/8.5/35/1.5/10 of INT-A002/DSPC/chol/PEG-DMG/prodrugs) with nucleic acids in aqueous buffer at a volumetric flow rate ratio of 1:3 (ethanol to aqueous, combined flow rate 28 ml/min) at room temperature. The product was then dialyzed against 1× phosphate-buffered saline (PBS) at pH 7.4 for 24 hours to remove residual ethanol and to raise the pH.

The physiochemical properties of the LNPs prepared as described above were subsequently characterized. Particle size was determined by dynamic light scattering using a Malvern Zetasizer Nano ZS (Malvern, UK) following buffer exchange into phosphate-buffered saline. Number-weighted size and distribution data was used. Lipid concentrations were determined by measuring total cholesterol using the Cholesterol E enzymatic assay kit from Wako Chemicals USA (Richmond, VA). mRNA entrapment was determined using a modified Quanti-iT Ribogreen assay (ThermoFisher, Waltham, MA). LNP-mRNA systems were incubated in the presence or absence of 1% Triton X-100 (Sigma-Aldrich, St. Louis, MO). Fluorescence intensities (Ex/Em: 480/520 nm) were compare to determine % mRNA entrapment.

### Example 1: LNP Efficiently Accumulate in APCs in the Pancreatic Islets, Pancreatic Lymph Nodes and Spleen

This example demonstrates that LNPs provide a potential delivery platform to effectively deliver drugs and antigens to the APCs located in a subject. This may have the additional effect of limiting side-effects and reducing dose requirements by avoiding drug accumulation in other cell types and providing effective delivery to the APC populations.

In order to determine LNP accumulation in pancreatic APCs, mice received 2 injections, 24 hours apart, of LNP containing the fluorescent marker DiO injected at a dose of 600 mg/kg i.p. At 48 hours following the first injection, animals were euthanized and pancreatic islets and lymph nodes were harvested. Islets were hand-picked to 99% purity. Islet and lymph nodes were dispersed into single cell suspensions and stained for viability, CD45 (pan-immune cell marker) and CD11b and CD11c (APC markers), and the number of DiO positive cells was quantified by flow cytometry.

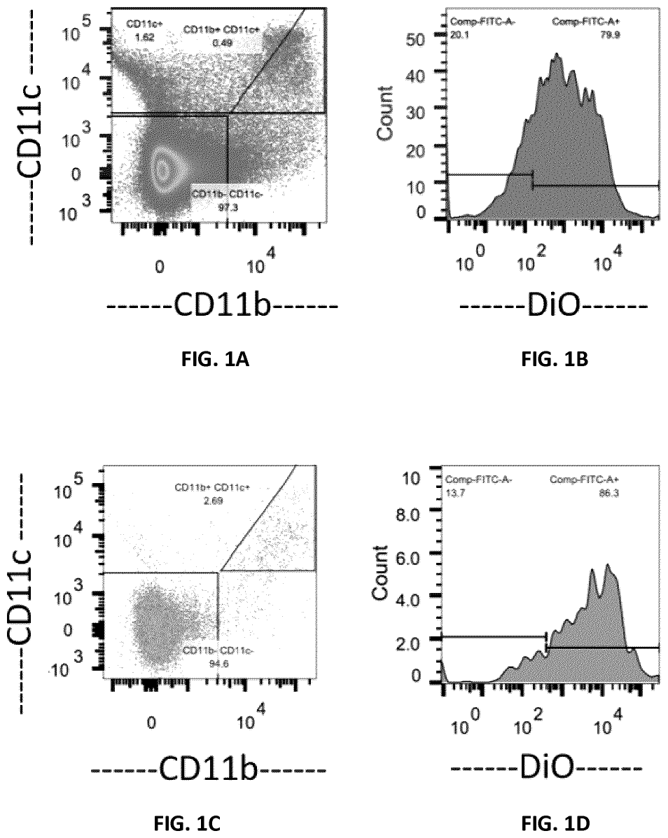

FIGS. 1A-D shows that through a simple injection, LNPs are taken up by APCs in both pancreatic islets and pancreatic lymph nodes. Furthermore, as shown in both the lymph node and islet, there is limited LNP uptake by other cell types, including endocrine cells in the islets, and non-APC immune cells (e.g., T cells) in the lymph node (Table 1).

TABLE 1
LNPs specifically accumulate in pancreatic APCs

| Tissue | Cell type | DiO+ (% total population) |
|---|---|---|
| Pancreatic | Islet Macrophages | 88 ± 3 |
| islets | All other islet cells | 0.84 ± 0.5 |
| Pancreatic | Dendritic cells | 74 ± 5 |
| lymph nodes | Lymphocytes | 9.7 ± 4 |

n = 2-3 biological samples, 2-3 mice pooled per sample to obtain sufficient cell numbers

In order to compare the effect of LNP lipid composition on islet APC targeting, C57Bl/6J male mice were injected with 150 mg/kg DSPC/Chol or ionizable LNP with DiO, 24 hours prior to islet, pancreatic lymph node, and splenocyte isolation. Islets were hand-picked to 99% purity. Tissues were dispersed into single cell suspensions and stained for viability, CD45 (pan-immune cell marker) and CD11b and CD11c (APC markers), and the number of DiO positive cells was quantified by flow cytometry.

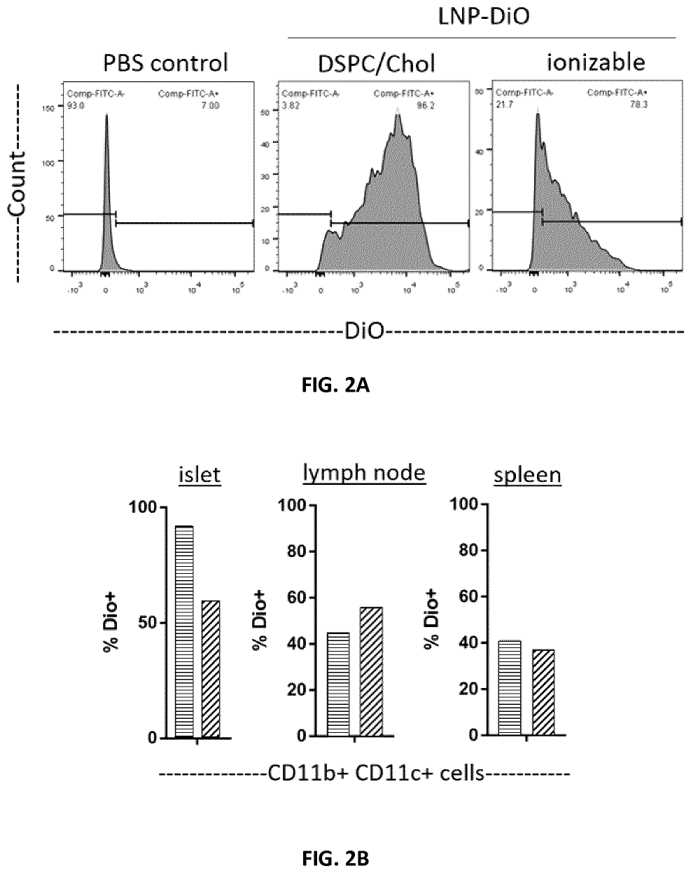

The pronounced accumulation in APCs is observed for both liposomes and LNP (DSPC/Chol or ionizable) in the islet, pancreatic lymph node and spleen (FIG. 2).

### Example 2: LNP can Efficiently Co-Encapsulate and Stably Retain Lipid Conjugates (Prodrugs) of Tolerizing Agents

As shown in Example 1, both liposomes and LNP (DSPC/Chol and ionizable) can accumulate in APCs in vivo. Another non-limiting aspect of the disclosure provides LNPs with two or more different immunomodulatory agents co-formulated therein. The prodrugs are uniquely suited for co-formulation due to their lipophilic nature. The results not only show that more than one prodrug can be formulated in an LNP at high encapsulation efficiency, but also that the prodrugs can be stably retained within the LNPs. Moreover, the results herein show that the prodrug strategy is uniquely suited for the delivery of combination ratios of two or more immunomodulatory agents.

As shown in FIG. 3, both dexamethasone (D045) and calcitriol (D053, D068, D083) lipid prodrugs are stably retained within LNP after 2 hours of incubation in human plasma. While this example demonstrates the ability to prepare LNP containing up to 20 mol % of dexamethasone and calcitriol prodrugs, the LNPs of the disclosure have been shown to be capable of incorporating up to 99 mol % of the prodrugs. Therefore, using the prodrug strategy of the disclosure, co-formulation of additional immunomodulatory agents (e.g., such as but not limited to acetylsalicylic acid, mycophenolate, sirolimus and tacrolimus) can be achieved in the LNP formulations disclosed herein.

The results showing efficient entrapment of different lipid prodrugs in LNPs are set forth in Table 2. Tables 3, 4 and 5 below demonstrate that different ratios of these prodrugs can be achieved with no impact on entrapment efficiencies.

TABLE 2
Different lipid conjugates and combinations of
lipid conjugates can be formulated into LNP

| Dexamethasone | Calcitriol | | | % Entrap |
|---|---|---|---|---|
| Prodrug | Prodrug | Size | PdI | (Dex/Calcitriol) |
| — | — | 43 | 0.054 | N/A |
| — | D053 | 55 | 0.070 | —/98 |
| — | D068 | 43 | 0.073 | —/94 |
| — | D083 | 51 | 0.066 | —/97 |
| — | D087 | 40 | 0.055 | —/99 |
| D034 | — | 50 | 0.043 | 97/— |
| D034 | D052 | 54 | 0.050 | 93/92 |
| D034 | D083 | 49 | 0.079 | 94/100 |
| D045 | — | 47 | 0.051 | 96/— |
| D045 | D053 | 49 | 0.053 | 93/97 |
| D045 | D068 | 44 | 0.065 | 98/94 |
| D045 | D083 | 50 | 0.047 | 100/99 |
| D045 | D087 | 43 | 0.085 | 99/99 |

See WO 2020/191477 (PCT/CA2020/000039; incorporated herein by reference) for structures of the dexamethasone and calcitriol lipid prodrugs and FIGS. 10A-G herein.

TABLE 3
D045 and D053 Combination LNP

| | | | % Entrapment |
|---|---|---|---|
| D045:D053 | Size | PdI | (Dex/Calcitriol) |
| 10:10 | 51 | 0.051 | 100/100 |
| 3:10 | 53 | 0.058 | 100/97 |
| 1:10 | 51 | 0.065 | 93/97 |
| 10:3 | 49 | 0.049 | 100/98 |
| 10:1 | 48 | 0.037 | 99/94 |
| 3:3 | 52 | 0.069 | 94/88 |
| 1:1 | 56 | 0.047 | 100/97 |
| Control (0:0) | 44 | 0.037 | N/A |

TABLE 4
D045 and D068 Combination LNP

| | | | % Entrapment |
|---|---|---|---|
| D045:D068 | Size | PdI | (Dex/Calcitriol) |
| 10:10 | 47 | 0.055 | 99/98 |
| 3:10 | 45 | 0.059 | 94/95 |
| 1:10 | 44 | 0.054 | 95/98 |
| 10:3 | 48 | 0.054 | 99/96 |
| 10:1 | 47 | 0.055 | 99/74 |
| 3:3 | 46 | 0.107 | 95/86 |
| 1:1 | 51 | 0.035 | 95/86 |
| Control (0:0) | 45 | 0.041 | N/A |

TABLE 5
D045 and D083 Combination LNP

| | | | Entrapment |
|---|---|---|---|
| D045:D083 | Size | PdI | (Dex/Calcitriol) |
| 10:10 | 53 | 0.034 | 98/98 |
| 3:10 | 55 | 0.046 | 85/97 |
| 1:10 | 55 | 0.050 | 100/98 |
| 10:3 | 51 | 0.044 | 97/91 |
| 10:1 | 48 | 0.055 | 96/97 |
| 3:3 | 54 | 0.049 | 97/83 |
| 1:1 | 59 | 0.033 | 100/76 |
| Control (0:0) | 45 | 0.053 | N/A |

### Example 3: Tolerization of APCs with LNP-Encapsulated Calcitriol and Dexamethasone Prodrugs

This example demonstrates that the prodrug LNPs of the disclosure are able to tolerize APCs ex vivo.

Bone-marrow derived dendritic cells (BMDCs) were treated with LNPs containing various calcitriol and dexamethasone prodrugs (alone or in combination) for 48 hours. Subsequently, BMDCs were challenged with lipopolysaccharide (LPS) stimulation for 24 hours to determine whether prodrug formulations could prevent LPS-mediated activation (i.e., tolerize BMDCs).

In particular, murine bone-marrow cells were differentiated into APCs (in this case dendritic cells) following standard procedures. Briefly, bone-marrow derived dendritic cells (BMDCs) were generated from bone marrow isolated from C57Bl/6 male mice (Charles River), by culturing in RPMI 1640 media (supplemented with 10% FBS, 10 mM HEPES, 50 μM β-mercaptoethanol, 1× GlutaMAX, 0.1 mM non-essential amino-acids, 1% Pen/Strep, 30 ng/ml GM-CSF, and 30 ng/ml IL-4). After 5 days of differentiation, BMDCs were treated for 48 hours with LNP-encapsulated prodrugs or empty-LNP (LNP control) or vehicle, and subsequently activated by addition of lipopolysaccharide (LPS, 10 ng/ml) for 24 hours. BMDCs were then harvested for downstream analyses.

To characterize the cell surface markers for BMDC (described above), floating and loosely adherent cells were collected and washed twice with PBS. 500,000 cells were incubated with Fc blocker (Invitrogen CD16, CD32 Cat #14-016186) at 1 μg/sample for 10 min at room temperature. CD80-PE (BioLegend cat #104707), CD86-PE-Cy7 (BioLegend cat #105013), MHC II-APC (BioLegend cat #107613), Fixable Viability-eF780 (Invitrogen cat #65-0865-14), and CD11c-BV510 (BioLegend cat #117337) were added to samples according to manufactures recommendations and incubated for 20 minutes at room temperature. Samples were then washed twice with FACS buffer (PBS, 2% FBS) prior to analysis by flow cytometry.

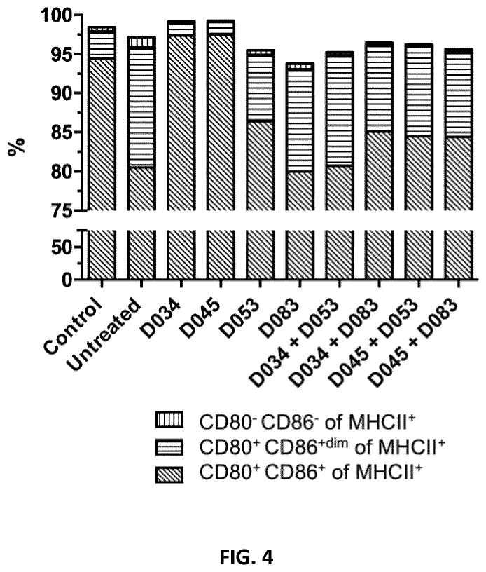

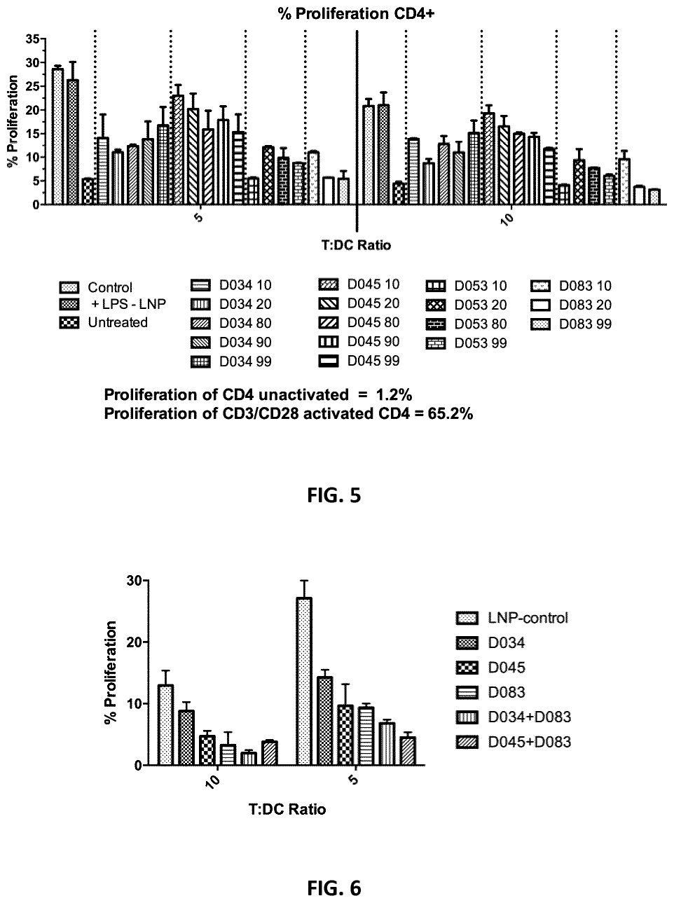

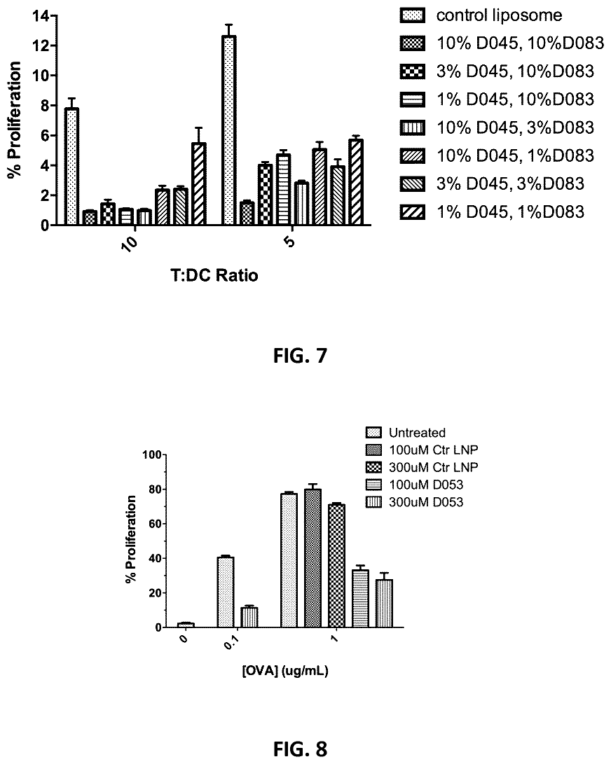

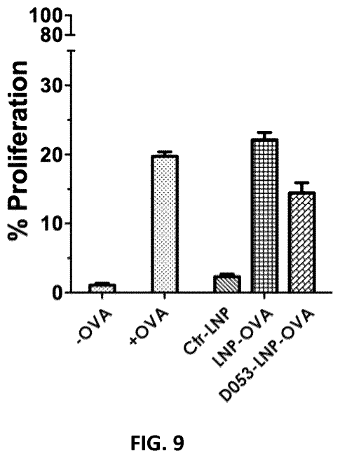

As shown in FIG. 4, prodrug treatment effectively tolerized BMDCs ex vivo. Various tolerizing prodrugs and prodrug combinations inhibited LPS-induced expression of B7 molecules (CD80 and CD86) on the cell surface of DCs, consistent with the induction of a tolerogenic or immature phenotype. Activated DCs have high expression of MHCII and B7 molecules while tolerogenic/immature DCs can have reduced B7 expression. Furthermore, as shown in FIG. 5, single prodrug treatments induced functionally tolerogenic DCs in allogeneic mixed leukocyte reaction with complete prevention of proliferation observed in some formulations. Notably, this is a very robust type of immune response (akin to organ transplant rejection), underlining the efficacy of these tolerizing prodrugs. When combinations of these immunomodulatory agents were used, greater tolerance or suppression of the T cell proliferation was observed (FIGS. 6 and 7).

### Example 4: Prodrug-LNP can Reduce CD4+ T-Cell Proliferation in an Antigen-Specific Mouse Model

Previous demonstrations of tolerizing APCs were achieved in an allogenic mouse model where APC and T cells were from MHC mismatched donors. However, to demonstrate the ability of prodrugs to reduce antigen-specific stimulation, an antigen specific mouse model was used. CD4 T cells were collected from OT-II mice. These mice have T cell receptors on CD4+ T cells engineered to bind and react to Ovalbumin 323-339 peptide fragment (OVA) when presented on I-Ab MHC Class II. When APCs are loaded with OVA and mixed with these T cells, a robust proliferation of T cells is observed (akin to having an antigen-specific immune disorder).

For the antigen specific model, splenocytes were isolated from OT-II mice (B6.Cg-Tg (TcraTcrb) 425Cbn/J; Jackson Laboratories). CD4+ T cells were purified by CD4 positive selection (EasySep™ Mouse CD4+ T Cell Isolation Kit, Catalog #19852, Stem Cell Technologies) and labelled with 10 μM CFSE and plated in 96-well U bottom plates at a density of 100,000 cells/well. On the same day, prodrug treated C57BL/6 BMDCs (described above) were harvested, pulsed with/without OVA peptide, irradiated at 30 Gy, and co-cultured with the CD4+ cells for 3 days at T:DC ratios of 50:1, 10:1 and 5:1, in RPMI 1640 media (supplemented with 10% FBS, 10 mM HEPES, 50 μM β-mercaptoethanol, 10 mM sodium pyruvate, 1× GlutaMAX, and 1% Pen/Strep). Subsequently, cells were stained for viability (eBioscience Fixable Viability Dye eFlour780, cat #65-0865) and CD4 (eBioscience CD4 Monoclonal Antibody (RM4-5), cat #48-0042-82), and T cell proliferation was quantified via CFSE dilution by flow cytometry.

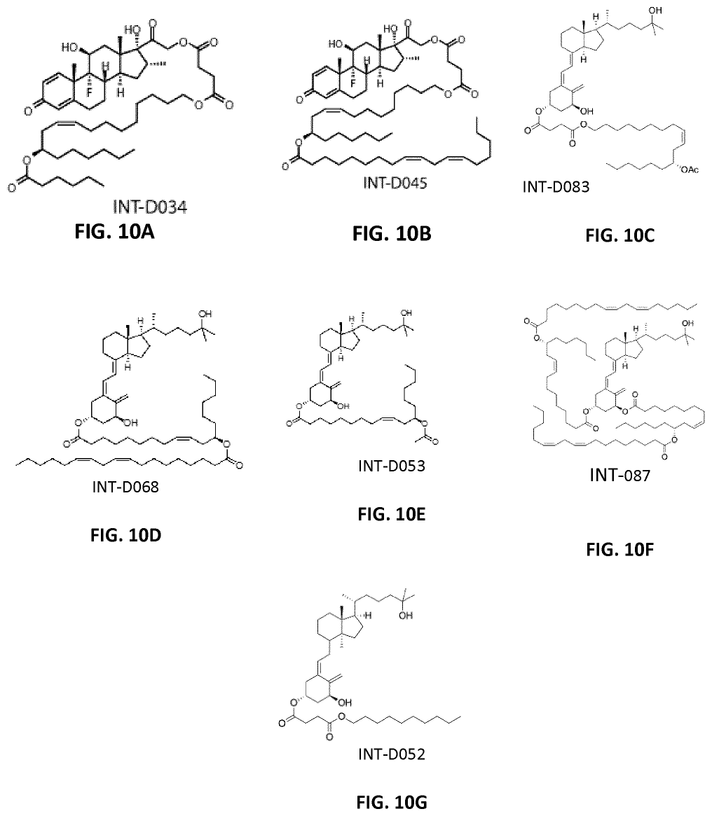

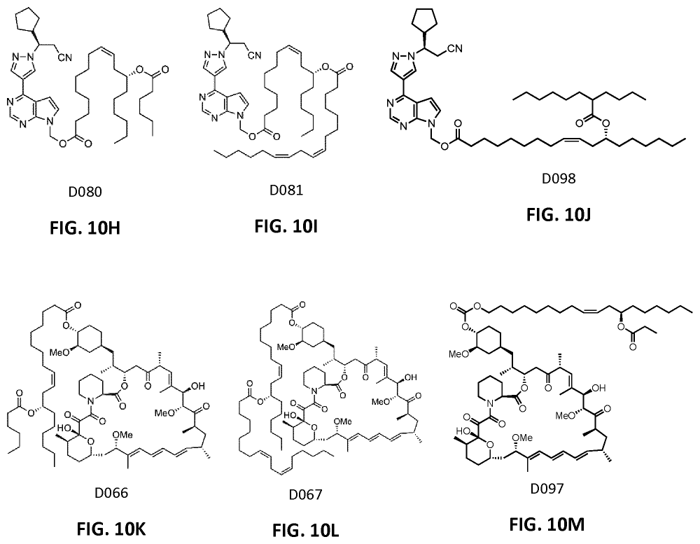

As shown in FIG. 8, D053 significantly reduces the amount of T cell proliferation induced by OVA loaded BMDCs. Furthermore, this response is conserved when the OVA peptide is co-formulated into LNP that contain tolerizing prodrugs (FIG. 9).

### Example 5: Reduction in CD4+ T-Cell Proliferation is Observed During Prodrug-LNP and LNP-mRNA Co-Treatment

In the previous example, immunomodulatory agent-lipid conjugates (either in separate LNP or coloaded into the same LNP as the peptide antigen) were shown to be able to suppress Ova specific CD4+ T-cell proliferation. In addition to peptides or proteins, mRNA which is translated into its encoded protein and subsequently processed along the antigen presentation pathway, can be used as a source of antigen.

The mRNA used in this example is modified to minimize immune stimulation and encodes for full length Ovalbumin (Ova) protein (Trilink, L-7210). Bone marrow-derived dendritic cells (BMDCs) were prepared as previously described and co-cultured with isolated CD4+ T cells (OT-II T cells) that specifically recognize the Ova323-339 epitope loaded in H-2b MHC Class II (C57BL/6 haplotype). Proliferation was assessed based on CFSE dilution by flow cytometry. BMDCs pulsed for 4 hours with free Ova323-339 peptide (0.1 μg/mL) served as an antigen-loaded control (Free Ova Peptide), and untreated BMDCs served as the No Antigen control. 48 hours prior to co-culturing, BMDCs were treated with LNP-mRNA only or LNP-mRNA and LNP containing dexamethasone (D034) or calcitriol (D053) prodrugs.

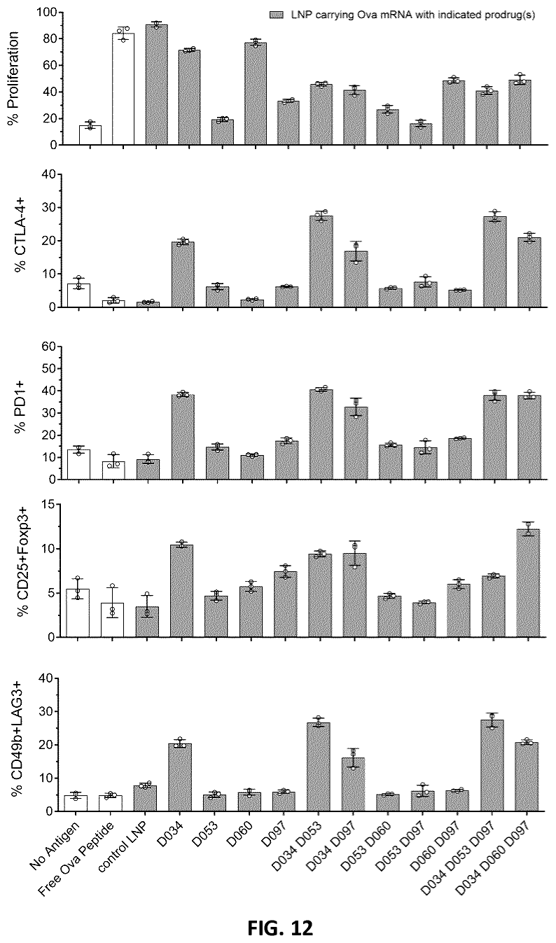

As shown in FIG. 11, LNP-mRNA induced similar levels of T-cell proliferation as free peptide antigen. The level of proliferation was suppressed when BMDCs were cotreated with LNP containing lipid conjugates D034 or D053. This further demonstrates that prodrug-LNP (i.e. lipid conjugate-LNP) can be used to reduce antigen specific T-cell proliferation regardless of whether the antigen is a peptide (FIG. 8) or mRNA (FIG. 11).

### Example 6: LNP can Efficiently Co-Encapsulate mRNA and Prodrugs of Tolerizing Agents

In this example, the ability to co-load mRNA and immunomodulatory agent-lipid conjugates within the same LNP is demonstrated. Up to three distinct prodrugs and up to 10 mol % is shown in Table 6. There was little impact on particle diameter (Z-Ave), polydispersity (PDI) or mRNA entrapment. Prodrug entrapment was also unaffected (Table 7).

TABLE 6
Co-entrapment of prodrugs in LNP-mRNA do not
affect LNP parameters or mRNA entrapment

| Lipid Conjugate (%) | Z-Ave (nm) | PDI | % mRNA Entrap. |
|---|---|---|---|
| N/A | 76 | 0.019 | 98 |
| D034 (10%) | 77 | 0.040 | 97 |
| D053 (10%) | 69 | 0.034 | 97 |
| D060 (10%) | 79 | 0.019 | 95 |
| D097 (10%) | 86 | 0.058 | 93 |
| D034/D053 (5 + 5%) | 71 | 0.028 | 97 |
| D034/D053 (9.9 + 0.1%) | 71 | 0.024 | 96 |
| D034/D097 (5 + 5%) | 72 | 0.021 | 95 |
| D053/D060 (5 + 5%) | 72 | 0.025 | 97 |
| D053/D097 (5 + 5%) | 75 | 0.021 | 95 |
| D060/D097 (5 + 5%) | 74 | 0.011 | 93 |
| D034/D053/D097 | 73 | 0.035 | 96 |
| (3.3 + 3.3 + 3.3%) | | | |
| D034/D060/D097 | 73 | 0.029 | 95 |
| (3.3 + 3.3 + 3.3%) | | | |

TABLE 7
LNP Can efficiently co-encapsulate mRNA
and lipid conjugate of tolerizing agents

| | Z-ave | | % mRNA | % Prodrug |
|---|---|---|---|---|
| Prodrug | (nm) | PdI | Entrap. | Entrap. |
| — | 70 | 0.017 | 98 | N/A |
| D034/D053/D060 | 69 | 0.026 | 97 | 97/100/99 |
| D034/D097 | 76 | 0.048 | 97 | 100/97 |
| D034/D053 | 70 | 0.021 | 95 | 100/100 |

### Example 7: LNPs Coloaded with mRNA Antigen and Immunomodulatory Agent-Lipid Conjugates Elicit Distinct Tolerogenic Mechanisms in CD4 T Cells

Upon recognition of an antigen presented by antigen presenting cells, several responses in T cells can contribute to antigen-specific immune tolerance, including changes in T cell function (e.g. anergy or exhaustion), suppressed expansion or deletion of antigen-specific effector T cells, and the expansion of antigen-specific regulatory T cells including Foxp3+ Tregs and IL-10-producing Trls. To determine whether treatment of antigen presenting cells with LNPs coloaded with antigen-encoding mRNA and prodrugs can induce tolerogenic responses in antigen-specific T cells, the phenotypes in Ova-reactive OT-II CD4+ T cells in response to co-culture with antigen presenting cells (BMDCs) that were pre-treated with LNPs coloaded with Ova mRNA and various lipid conjugates was studied.

BMDCs were generated from C57BL/6 donor mice and treated for 48 hours with LNP formulations (or untreated) and matured with 10 ng/mL LPS for the final 24 hours. Full length Ova-encoding mRNA in LNP formulations in this example is modified to minimize immune stimulation (L-7210, TriLink) and BMDCs pulsed for 4 hours with free Ova323-339 peptide (0.1 μg/mL) served as an antigen-loaded control. Splenic CD4+ T cells were isolated from OT-II mice (I-Ab: Ova323-339 specific TCR transgenic mice, Jackson Laboratory) using a CD4 Easy Sep Kit (Stem Cell Technologies) and labelled with CFSE. On the day of coculture, BMDCs were washed 2× and co-cultured with CD4 T cells at a ratio of 1:5 BMDC:CD4 cells at a density of 100,000 CD4 cells per well. T cells were stained after 3 days of co-culture with the various markers (fixable viability dye, CD4, PD1, CTLA4, CD25, Foxp3, CD49b, and LAG3) and assessed by flow cytometry.

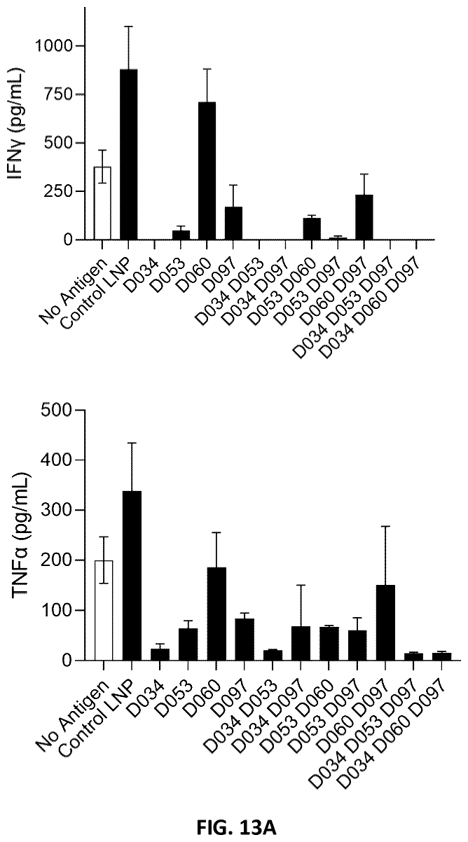

As shown in FIG. 12, OT-II CD4+ T cells proliferated greatly in response to BMDCs pre-treated with Ova mRNA only LNPs (comparable to induction by BMDCs loaded with free Ova 323-339 peptide) while OT-II CD4+ T cells had minimal proliferation in response to untreated (no antigen) BMDCs. Moreover, in the absence of immunomodulatory agent-lipid conjugates, there was no change in the frequencies of CD49b+LAG3+ (Trl marker) cells, CD25+Foxp3+ (Treg marker) cells, and no induction of PD1+ and CTLA-4+ cells amongst OT-II CD4+ cells stimulated with Ova antigen presenting BMDCs. In contrast, BMDCs pre-treated with Ova mRNA- and lipid conjugate-coloaded LNPs, elicited changes in various CD4+ T cell responses; several LNP formulations reduced antigen-specific proliferation (top panel) indicative of reduced effector CD4+ T cell expansion; other LNP formulations increased the frequency of CD4+ cells expressing CTLA-4 and PD1 (second and third panel); while other formulations markedly induced Treg (CD25+Foxp3+, fourth panel) or Trl (CD49b+LAG3+, final panel) frequencies. Thus, LNPs coloaded with both modified mRNA-encoded antigen and immunomodulatory lipid conjugates can induce BMDCs to elicit distinct tolerogenic responses in antigen-specific CD4+ T cells (anergy, regulatory cell expansion, limited effector cell expansion).

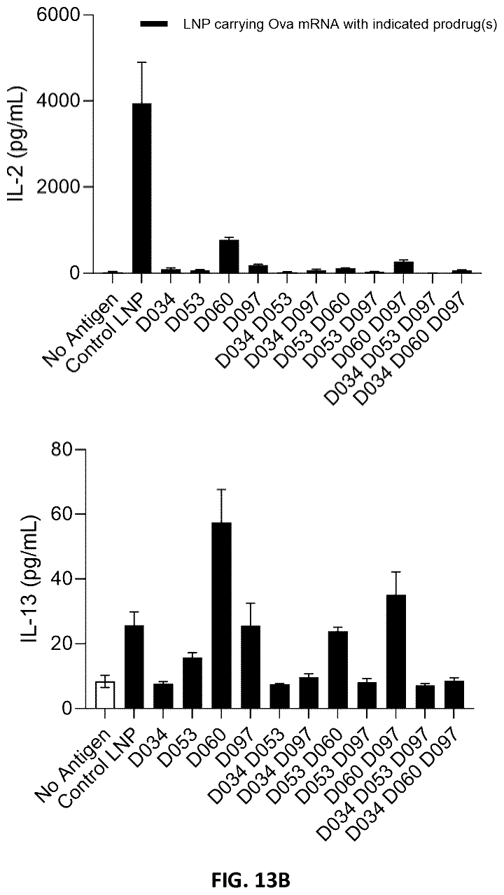

FIGS. 13A and 13B show that while delivery of mRNA-encoded antigen alone in an LNP caused an induction of Th1 and Th2 cytokine secretion (despite being modified to minimize innate immune response to mRNA), codelivery of various immunomodulatory prodrugs inhibits this response, and in many cases even suppresses it below baseline levels (as in IFNγ and TNFα). Therefore, codelivery of immunomodulatory prodrugs with modified mRNA-encoded antigen to BMDCs reduces antigen-specific Th1 and Th2 responses.

### Example 8: LNPs Coloaded with mRNA-Encoded Antigen and Immunomodulatory Lipid Conjugates can Induce Antigen-Specific Tolerance In Vivo

This example demonstrates that LNPs coloaded with modified mRNA-encoded antigen and immunomodulatory lipid conjugates can induce antigen-specific tolerance in vivo.

Female C57BL/6J mice were injected with LNPs loaded with modified Ova mRNA (L-7210, TriLink) with/without D034 coloaded in the same particles. Mice were injected ip once biweekly for a total of 3 injections of LNPs (dosed as 10 μg mRNA/mouse) or buffer only. Ova specific IgG1 antibodies were measured from serum collected 2 weeks after the final injection by mouse anti-Ova IgG1 ELISA (Cayman Chemical, cat #500830-96).

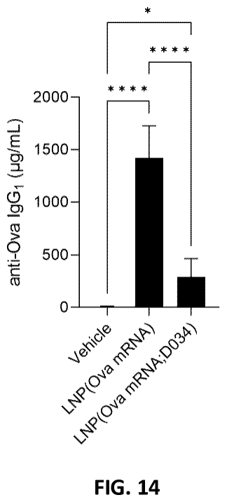

As shown in FIG. 14, injection of LNPs carrying Ova mRNA in mice induce high levels of anti-Ova antibodies in serum, indicative of an antigen specific immune response to Ova antigen. In contrast, mice receiving LNPs coloaded with Ova mRNA and lipid conjugate D034 had robustly suppressed anti-Ova IgG1 levels. Thus, the humoral immune response to Ova antigen encoded by modified mRNA has been reduced by coloading of immunomodulatory lipid conjugate in LNPs. Similar results (not shown), were observed for LNP co-formulated with D034 in combination with additional lipid conjugates (D034+D053, and D034+D053+D097).

Although the invention has been described and illustrated with reference to the foregoing examples, it will be apparent that a variety of modifications and changes may be made without departing from the invention.

1. An immunomodulatory combination comprising:
a lipid conjugate comprising an immunomodulatory agent covalently linked to a lipophilic moiety by a cleavable linkage or through a cleavable linker; and
an antigen and/or one or more nucleic acids that encode the antigen, wherein the antigen is a protein, polypeptide, peptide, lipoprotein, glycolipid, polynucleotide, or polysaccharide,
wherein the lipid conjugate and the antigen and/or the one or more nucleic acids that encode the antigen are formulated in separate delivery vehicles or co-formulated in the same delivery vehicle.

2. The immunomodulatory combination as claimed in claim 1, wherein the delivery vehicles are lipid nanoparticles and/or liposomes,
optionally wherein the delivery vehicles are lipid nanoparticles which deliver to antigen presenting cells (APCs).

3. The immunomodulatory combination as claimed in claim 2, wherein:
the antigen or the one or more nucleic acids encoding the antigen is entrapped within a lipid nanoparticle or liposome and has a net charge that is opposite a net charge of a lipid in the lipid nanoparticle, or
the antigen is lipophilic and incorporated into a lipid compartment of a lipid nanoparticle or liposome, or
wherein the antigen is hydrophilic and entrapped in a liposome containing an aqueous core.

4. The immunomodulatory combination as claimed in claim 1, wherein the lipid conjugate is co-formulated in the same delivery vehicle as the antigen or the one or more nucleic acids that encode the antigen.

5. The immunomodulatory combination as claimed in claim 1, wherein the lipid conjugate is a first lipid conjugate, and the immunomodulatory combination further comprises a second lipid conjugate, wherein the second lipid conjugate comprises an immunomodulatory agent covalently linked to a lipophilic moiety by a cleavable linkage or through a cleavable linker, wherein the immunomodulatory agent of the second lipid conjugate is different from the immunomodulatory agent of the first lipid conjugate, wherein the first lipid conjugate and the second lipid conjugate are formulated in separate delivery vehicles or are co-formulated in the same delivery vehicle,
optionally wherein the immunomodulatory combination comprises a plurality of lipid conjugates, wherein the plurality of lipid conjugates comprise 3, 4, 5, 6, 7, 8, 9, or 10 immunomodulatory agents, wherein each immunomodulatory agent of the plurality of lipid conjugates is different, and wherein each lipid conjugate of the plurality of lipid conjugates is independently formulated in a separate delivery vehicle from the other components of the immunomodulatory combination or is co-formulated with one or more of the other components of the immunomodulatory combination.

6. The immunomodulatory combination as claimed in claim 5, wherein the immunomodulatory agent of the second lipid conjugate targets a different immune pathway than the immunomodulatory agent of the first lipid conjugate.

7. The immunomodulatory combination as claimed in claim 5, wherein the first lipid conjugate and the second lipid conjugate are co-formulated in the same delivery vehicle.

8. The immunomodulatory combination as claimed in claim 1, wherein each immunomodulatory agent is a tolerogenic agent or anti-inflammatory agent.

9. The immunomodulatory combination as claimed in claim 1, wherein each immunomodulatory agent is an immunostimulant or an immunosuppressant.

10. The immunomodulatory combination as claimed in claim 1, wherein each immunomodulatory agent is independently: a non-steroidal anti-inflammatory drug (NSAID), an inflammasome inhibitor, a Janus kinase (JAK) inhibitor, a corticosteroid, an mTOR inhibitor, a DMARD (disease-modifying antirheumatic drug), a calcineurin inhibitor, or a vitamin D receptor agonist.

11. The immunomodulatory combination as claimed in claim 1, wherein each immunomodulatory agent is independently prednisone, budesonide, prednisolone, methylprednisolone, hydrocortisone, cortisone, betamethasone, budesonide, triamcinolone, flunisolide, beclomethasone, fluticasone, mometasone, fludrocortisone, flumethasone, triamcinolone acetonide, isoflupredone, corticosterone, desoxycortone acetate, desoxycortone enanthate, 11-deoxycorticosterone, 11-deoxycortisol, aldosterone, dexamethasone, calcitriol, acetylsalicylic acid, salicylate, mycophenolic acid, sirolimus, tacrolimus, cholecalciferol, calcifediol, alfacalcidol, calcipotriol, falecalcitriol, maxacalcitol, paricalcitol, doxercalciferol, 22-oxacalcitriol, tacalcitol, eldecalcitol, elocalcitol, inecalcitol, becocalcidiol, seocalcital, ergocalciferol, lexacalcitol, retinoic acid, cyclophosphamide (nitrogen mustards), filgotinib, baricitinib, tofacitinib, ruxolitinib, upadacitinib, oclacitinib, peficitinib, fedratinib, delgocitinib, deucravacitinib, abrocitinib, auranofin, apremilast, azathioprine, chloroquine, hydroxychloroquine, ciclosporin, leflunomide, methotrexate, minocycline, sulfasalazine, salicylic acid, diflunisal, salsalate, naproxen, ibuprofen, oxaprozin, loxoprofen, zaltoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, celecoxib, firocoxib, parecoxib, etoricoxib, clonixin, licofelone, MCC950, glyburide, CY-09, tranilast, oridonin, BOT-4-one (2-cyclohexylimino-6-methyl-6,7-dihydro-5H-benzo[1,3]oxathiol-4-one), INF39 (Ethyl 2-(2-chlorobenzyl) acrylate), MNS (3,4-Methylenedioxy-β-nitrostyrene), fenamic acid, beta-hydroxybutyric acid, quercetin, JC-171, ibrutinib, OLT1177, FC11A-2, INF58, JC124, or Ethyl 2-((2-chlorophenyl) (hydroxy)methyl) acrylate.

12. The immunomodulatory combination as claimed in claim 1, wherein the antigen is a first antigen, and the immunomodulatory combination further comprises a second antigen or one or more nucleic acids encoding the second antigen, wherein the first antigen is different than the second antigen,
wherein the second antigen or the one or more nucleic acids encoding the second antigen is formulated in a separate delivery vehicle from the other components of the immunomodulatory combination or is co-formulated with one or more of the other components of the immunomodulatory combination,
optionally wherein the immunomodulatory combination comprises a plurality of antigens or comprises one or more nucleic acids encoding the plurality of antigens, or comprises a combination of antigens and antigen-encoding nucleic acid(s) for providing the plurality of antigens, wherein the plurality of antigens comprises 3, 4, 5, 6, 7, 8, 9, or 10 antigens, and each antigen or antigen-encoding nucleic acid is independently formulated in a separate delivery vehicle from the other components of the immunomodulatory combination or is co-formulated with one or more of the other components of the immunomodulatory combination.

13. The immunomodulatory combination as claimed in claim 12, wherein the one or more nucleic acids encoding the first antigen and the one or more nucleic acids encoding the second antigen are comprised within a single nucleic acid and the first antigen and the second antigen are co-formulated in the same delivery vehicle.

14. The immunomodulatory combination as claimed in claim 1, for use in treatment of a subject having an antigen-induced disorder or undesired antigen-driven immune response, or for use in manufacture of a medicament for treating the subject,
optionally wherein the antigen-induced disorder or undesired antigen-driven immune response is selected from: autoimmune diseases (T cell and/or antibody responses to self antigen), allergic diseases (T cell and IgE responses to environmental or food antigens), transplantation (T cell responses against major and minor histocompatibility antigens in donor tissue/organ/cell), anti-drug antibody responses (antibody responses that diminish efficacy of therapeutics), gene/protein replacement therapy (T cell/antibody response against proteins therapeutically replaced in genetic protein deficiencies),
optionally wherein the antigen-induced disorder or undesired antigen-driven immune response is selected from: multiple sclerosis, rheumatoid arthritis, myelin oligodendrocyte glycoprotein antibody disorder, vitiligo, type 1 diabetes, primary biliary cholangitis, anti-GBM nephritis/Goodpasture's disease, celiac disease, psoriasis, myasthenia gravis, immune thrombocytopenia purpura, Grave's disease, neuromyelitis optica, pemphigus vulgaris, bullous pemphigoid, cicatricial pemphigoid, lupus including systemic lupus erythematosus SLE, autoimmune liver disease, myositis, Evan's syndrome, transverse myelitis, Guillain-Barré syndrome, warm autoimmune hemolytic anemia, chronic inflammatory demyelinating polyneuropathy, autoimmune dysautonomia, autoimmune angioedema, Hashimoto's thyroiditis, Lambert-Eaton syndrome, peanut/legume allergy, tree nut allergy (antigens from any of cashew, pistachio, hazelnut, walnut, almond), egg allergy, cow's milk allergy, soy allergy, fish allergy, shellfish allergy, sesame allergy, wheat allergy, allergic airway disease, and allergies caused by environmental allergens (antigens from pollen, dust, pet dander, mold and cockroaches).

15. A method for treating a subject having an antigen-induced disorder or undesired antigen-driven immune response comprising administering the immunomodulatory combination as defined in claim 1 to the subject, wherein the antigen, or the one or more nucleic acids encoding the antigen, and the lipid conjugate are administered together or sequentially,
optionally wherein the antigen-induced disorder or undesired antigen-driven immune response is selected from: autoimmune diseases (T cell and/or antibody responses to self antigen), allergic diseases (T cell and IgE responses to environmental or food antigens), transplantation (T cell responses against major and minor histocompatibility antigens in donor tissue/organ/cell), anti-drug antibody responses (antibody responses that diminish efficacy of therapeutics), or gene/protein replacement therapy (T cell/antibody response against proteins therapeutically replaced in genetic protein deficiencies),
optionally wherein the antigen-induced disorder is: multiple sclerosis, rheumatoid arthritis, myelin oligodendrocyte glycoprotein antibody disorder, vitiligo, type 1 diabetes, primary biliary cholangitis, anti-GBM nephritis/Goodpasture's disease, celiac disease, psoriasis, myasthenia gravis, immune thrombocytopenia purpura, Grave's disease, neuromyelitis optica, pemphigus vulgaris, bullous pemphigoid, cicatricial pemphigoid, lupus including systemic lupus erythematosus SLE, autoimmune liver disease, myositis, Evan's syndrome, transverse myelitis, Guillain-Barre syndrome, warm autoimmune hemolytic anemia, chronic inflammatory demyelinating polyneuropathy, autoimmune dysautonomia, autoimmune angioedema, Hashimoto's thyroiditis, Lambert-Eaton syndrome, peanut/legume allergy, tree nut allergy (antigens from any of cashew, pistachio, hazelnut, walnut, almond), egg allergy, cow's milk allergy, soy allergy, fish allergy, shellfish allergy, sesame allergy, wheat allergy, allergic airway disease, or allergies caused by environmental allergens (antigens from pollen, dust, pet dander, mold and cockroaches).
